(19) United States
(12) Patent Application Publication
(10) Pub. No.: US 2024/0158517 A1
(43) Pub. Date: May 16, 2024

(54) ANTI-HUMAN CXCR5 ANTIBODY AND USES THEREOF

(71) Applicant: HIFIBIO (HK) LIMITED, Hong Kong (CN)

(72) Inventors: Liang Schweizer, Cambridge, MA (US); Sami Ellouze, Cambridge, MA (US); Ayrin Kök Harunova, Cambridge, MA (US); Stéphanie Beq, Cambridge, MA (US); Nicola Arturo Aldo Beltraminelli, Cambridge, MA (US); Qian Zhang, Cambridge, MA (US); Francisco Adrian, Cambridge, MA (US); Yun-Yueh Lu, Cambridge, MA (US)

(21) Appl. No.: 18/280,927

(22) PCT Filed: Mar. 9, 2022
(86) PCT No.: PCT/US2022/019587

§ 371 Date: Sep. 7, 2023

Related U.S. Application Data
(60) Provisional application No. 63/158,462, filed on Mar. 9, 2021.

(30) Foreign Application Priority Data
Aug. 5, 2021 (WO) PCT/CN2021/111049

(51) Int. Cl. C07K 16/28; A61K 39/395; A61K 45/06; A61P 35/00; A61P 35/02; A61P 37/06; A61K 39/00

(52) U.S. Cl. CPC C07K 16/2866; A61K 39/3955; A61K 45/06; A61P 35/00; A61P 35/02; A61P 37/06; A61K 2039/505; C07K 2317/24; C07K 2317/52; C07K 2317/76; C07K 2317/92

(57) ABSTRACT

The invention provides monoclonal antibodies and antigen-binding fragments thereof specific for (human) CXCR5, and methods of using the same to treat Sjögren syndrome, certain cancers and autoimmune disorders, including combination therapy.

### REFERENCE TO RELATED APPLICATIONS

This application claims priority to U.S. Provisional Patent Application No. 63/158,462, filed on Mar. 9, 2021, and International Patent Application No. PCT/CN2021/111049, filed on Aug. 5, 2021, the entire contents of each of the above-references applications, including any drawings and sequence listings, are incorporated herein by reference.

### BACKGROUND OF THE INVENTION

C-X-C chemokine receptor type 5 (CXC-R5), also known as CD185 (cluster of differentiation 185) or Burkitt lymphoma receptor 1 (BLR1), is a G protein-coupled seven transmembrane receptor for chemokine CXCL13 (also known as BLC) and belongs to the CXC chemokine receptor family. In humans, the CXC-R5 protein is encoded by the CXCR5 gene, and it enables T cells to migrate to lymph node B cell zones.

The BLR1/CXCR5 gene is specifically expressed in Burkitt's lymphoma and lymphatic tissues, such as follicles in lymph nodes as well as in spleen. The gene plays an essential role in B cell migration. Through CXCL13 secretions, B cells are able to locate the lymph node. Additionally, some recent studies have suggested that CXCL13, through CXCR5, is capable of recruiting hematopoietic precursor cells (CD3⁻CD4⁺) which would cause the development of lymph nodes and Peyer's Patches.

On the other hand, T cells are unable to access B cell follicles without CXCR5 expression. This is a key step in the production of high affinity antibodies as B cells and T cells need to interact in order to activate the Ig class switch.

Thus CXCR5 has been shown to be expressed on mature resting B cells, tonsillar B cells, both CD4 and CD8 T cells, though it is often regarded as the defining marker for T Follicular Helper (Tfh) cells.

CXCR5 overexpression in breast cancer patients highly correlates with lymph node metastases. In addition, elevated CXCR5 expression may contribute to abnormal cell survival and migration in breast tumors that lack functional p53 protein. Minor allele of SNP rs630923, located in the area of CXCR5 gene promoter and associated with the risk of multiple sclerosis, is responsible for appearance of MEF2C-binding site resulted in reduced CXCR5 gene promoter activity in B-cells during activation, that could lead to decreased autoimmune response.

CXCR5 has also been linked to metastatic progression of prostate cancer—prostate cancer tissue as well as cell lines have been found to express higher non-basal levels of CXCR5. Furthermore, a correlation was found between the expression level of CXCR5 and Gleason score.

CXCR5 is required for the polarization/organization of GCs (germinal centers, Forster et al, 1996—Allen et al. 2004). CXCR5 and its ligand CXCL13 are required for the migration of B/T cells at the B/T border zone into GC of secondary lymphoid organs (Allen et al, 2004—Relf et al., 2012). The B/T cells interaction in the B/T zone is required for BCR affinity maturation and B cell expansion (Breitfield et al., 2000).

Most chemokines are known to bind to more than one receptor. However, the CXCR5-CXCL13 interaction appears to be unique in that no other ligands bind CXCR5 and no other receptors bind CXCL13, although CXCR3 is the closest paralog sharing about 38.5% amino acid sequence identity or 51.5% similarity with CXCR5.

Sjögren syndrome (SjS, SS) is a long-term autoimmune disease that affects the body's moisture-producing glands. The disease was named after Henrik Sjögren who described it in 1933. Primary symptoms of SS include a dry mouth and dry eyes. Other symptoms can include dry skin, vaginal dryness, a chronic cough, numbness in the arms and legs, feeling tired, muscle and joint pains, and thyroid problems. Those affected are also at an increased risk (5%) of lymphoma.

Between 0.2%-1.2% of the population are affected by SS, with half having the primary form (which occurs independently of other health problems) and half the secondary form (which is a result of another connective tissue disorder). Females are affected about ten times as often as males, and it commonly begins in middle age; but anyone can be affected. Among those without other autoimmune disorders, life expectancy is unchanged.

While the exact cause of SS is unclear, it is believed to involve a combination of genetics and an environmental trigger such as exposure to a virus or bacteria. The inflammation that results progressively damages the glands.

Current treatment of SS is directed at symptoms. Dry eyes treatment includes artificial tears, medications to reduce inflammation, punctal plugs, or surgery to shut the tear ducts. For a dry mouth, chewing gum (preferably sugar free), sipping water, or a saliva substitute may be used. In those with joint or muscle pain, ibuprofen may be used. Medications that can cause dryness, such as antihistamines, may also be stopped.

Thus, there is a need to develop therapeutic reagents that treats Sjögren syndrome, as well as other mechanistically related diseases or indications.

### SUMMARY OF THE INVENTION

The invention described herein provides antagonistic anti-hCXCR5 antibodies that also selectively depletes CXCR5⁺ cells through antibody dependent cell-mediated cytotoxicity (ADCC), which not only inhibits migration of both Tfh and B cells but also eliminates CXCR5⁺ cells already present, thus decreasing tissue damage and pathogenic antibody production.

Thus the invention described herein provides an isolated monoclonal antibody, or an antigen-binding fragment thereof, wherein said monoclonal antibody or antigen-binding fragment thereof is specific for human CXCR5 (hCXCR5), and wherein said monoclonal antibody comprises: (1) a heavy chain variable region (VH), comprising a VH CDR1 sequence, a VH CDR2 sequence, and a VH CDR3 sequence; wherein said VH CDR1 sequence, said VH CDR2 sequence, and said VH CDR3 sequence comprise any one of the VH CDR1, VH CDR2, and VH CDR3 sequences, respectively, in Tables A, B, and D; optionally, said VH CDR1 sequence, said VH CDR2 sequence, and said VH CDR3 sequence comprise the VH CDR1, VH CDR2, and VH CDR3 sequences, respectively, of any one of the monoclonal antibodies in Tables A, B, and D; and/or (2) a light chain variable region (VL), comprising a VL CDR1 sequence, a VL CDR2 sequence, and a VL CDR3 sequence; wherein said VL CDR1 sequence, said VL CDR2 sequence, and said VL CDR3 sequence comprise any one of the VL CDR1, VL CDR2, and VL CDR3 sequences, respectively, in Tables A, C, and D; optionally, said VL CDR1 sequence, said VL CDR2 sequence, and said VL CDR3 sequence comprise the VL CDR1, VL CDR2, and VL CDR3 sequences, respectively, of any one of the monoclonal antibodies in Tables A, C, and D.

In some embodiments, the VH CDR1 sequence, the VH CDR2 sequence, and the VH CDR3 sequence comprise the amino acid sequences of SEQ ID NOs: 1, 2, and 3, respectively, and the VL CDR1 sequence, the VL CDR2 sequence, and the VL CDR3 sequence comprise the amino acid sequences of SEQ ID NOs: 9, 10, and 11 respectively.

In some embodiments, the VH CDR1 sequence, the VH CDR2 sequence, and the VH CDR3 sequence comprise the amino acid sequences of SEQ ID NOs: 17, 18, and 19, respectively, and the VL CDR1 sequence, the VL CDR2 sequence, and the VL CDR3 sequence comprise the amino acid sequences of SEQ ID NOs: 25, 26, and 27 respectively.

In some embodiments, the VH CDR1 sequence, the VH CDR2 sequence, and the VH CDR3 sequence comprise the amino acid sequences of SEQ ID NOs: 33, 34, and 35, respectively, and the VL CDR1 sequence, the VL CDR2 sequence, and the VL CDR3 sequence comprise the amino acid sequences of SEQ ID NOs: 41, 42, and 43 respectively.

In some embodiments, the VH CDR1 sequence, the VH CDR2 sequence, and the VH CDR3 sequence comprise the amino acid sequences of SEQ ID NOs: 33, 49, and 51, respectively, and the VL CDR1 sequence, the VL CDR2 sequence, and the VL CDR3 sequence comprise the amino acid sequences of SEQ ID NOs: 57, 58, and 59 respectively.

In some embodiments, the VH CDR1 sequence, the VH CDR2 sequence, and the VH CDR3 sequence comprise the amino acid sequence of SEQ ID NOs: 33, 49, and 65, respectively, and the VL CDR1 sequence, the VL CDR2 sequence, and the VL CDR3 sequence comprise the amino acid sequence of SEQ ID NOs: 57, 58, and 59 respectively.

In some embodiments, the VH CDR1 sequence, the VH CDR2 sequence, and the VH CDR3 sequence comprise the amino acid sequences of SEQ ID NOs: 69, 70, and 71, respectively, and the VL CDR1 sequence, the VL CDR2 sequence, and the VL CDR3 sequence comprise the amino acid sequences of SEQ ID NOs: 76, 77, and 78 respectively.

In some embodiments, the VH CDR1 sequence, the VH CDR2 sequence, and the VH CDR3 sequence comprise the amino acid sequences of SEQ ID NOs: 33, 49, and 51, respectively, and the VL CDR1 sequence, the VL CDR2 sequence, and the VL CDR3 sequence comprise the amino acid sequences of SEQ ID NOs: 149, 150 and 151, respectively.

In some embodiments, the VH CDR1 sequence, the VH CDR2 sequence, and the VH CDR3 sequence comprise the amino acid sequences of SEQ ID NOs: 114, 115, and 116, respectively, and the VL CDR1 sequence, the VL CDR2 sequence, and the VL CDR3 sequence comprise the amino acid sequences of SEQ ID NOs: 120, 121, and 122 respectively.

In certain embodiments, the isolated monoclonal antibody or antigen-binding fragment thereof of the invention is a mouse-human chimeric antibody comprising constant region sequences of a human antibody (such as hIgG1, hIgG2, hIgG3, or hIgG4), wherein the VH sequence is any one of SEQ ID NOs: 8, 24, 40, 56, 65 or 75 or has at least 90%, 91%, 92%, 93%, 94%, 95%, 96%, 97%, 98%, 99% sequence identity to any one of the amino acid sequences of SEQ ID NOs: 8, 24, 40, 56, 65 or 75, and/or wherein the VL sequence is any one of SEQ ID NOs: 16, 32, 48, 63, 68 or 83 or has at least 90%, 91%, 92%, 93%, 94%, 95%, 96%, 97%, 98%, 99% sequence identity to any one of the amino acid sequences of SEQ ID NOs: 16, 32, 48, 63, 68 or 83.

In some embodiments, the VH sequence is SEQ ID NO: 8 or has at least 90%, 91%, 92%, 93%, 94%, 95%, 96%, 97%, 98%, 99% sequence identity to SEQ ID NO: 8, and the VL sequence is SEQ ID NO: 16 or has at least 90%, 91%, 92%, 93%, 94%, 95%, 96%, 97%, 98%, 99% sequence identity to SEQ ID NO: 16.

In some embodiments, the VH sequence is SEQ ID NO: 24 or has at least 90%, 91%, 92%, 93%, 94%, 95%, 96%, 97%, 98%, 99% sequence identity to SEQ ID NO: 24, and the VL sequence is SEQ ID NO: 32 or has at least 90%, 91%, 92%, 93%, 94%, 95%, 96%, 97%, 98%, 99% sequence identity to SEQ ID NO: 32.

In some embodiments, the VH sequence is SEQ ID NO: 40 or has at least 90%, 91%, 92%, 93%, 94%, 95%, 96%, 97%, 98%, 99% sequence identity to SEQ ID NO: 40, and the VL sequence is SEQ ID NO: 48 or has at least 90%, 91%, 92%, 93%, 94%, 95%, 96%, 97%, 98%, 99% sequence identity to SEQ ID NO: 48.

In some embodiments, the VH sequence is SEQ ID NO: 56 or has at least 90%, 91%, 92%, 93%, 94%, 95%, 96%, 97%, 98%, 99% sequence identity to SEQ ID NO: 56, and the VL sequence is SEQ ID NO: 63 or has at least 90%, 91%, 92%, 93%, 94%, 95%, 96%, 97%, 98%, 99% sequence identity to SEQ ID NO: 63.

In some embodiments, the VH sequence is SEQ ID NO: 65 or has at least 90%, 91%, 92%, 93%, 94%, 95%, 96%, 97%, 98%, 99% sequence identity to SEQ ID NO: 65, and the VL sequence is SEQ ID NO: 68 or has at least 90%, 91%, 92%, 93%, 94%, 95%, 96%, 97%, 98%, 99% sequence identity to SEQ ID NO: 68.

In some embodiments, the VH sequence is SEQ ID NO: 75 or has at least 90%, 91%, 92%, 93%, 94%, 95%, 96%, 97%, 98%, 99% sequence identity to SEQ ID NO: 75, and the VL sequence is SEQ ID NO: 83 or has at least 90%, 91%, 92%, 93%, 94%, 95%, 96%, 97%, 98%, 99% sequence identity to SEQ ID NO: 83.

In certain embodiments, the isolated monoclonal antibody or antigen-binding fragment thereof of the invention is a humanized antibody, optionally, wherein the humanized antibody comprises: (1) the VH sequence of any one of the monoclonal antibodies in Tables B, D and E or a VH sequence least 90%, 91%, 92%, 93%, 94%, 95%, 96%, 97%, 98%, 99% identical thereto; and/or the VL sequence of any one of the monoclonal antibodies in Tables C, D and E or a VL sequence least 90%, 91%, 92%, 93%, 94%, 95%, 96%, 97%, 98%, 99% identical thereto; (2) the VH sequence of SEQ ID NO: 96, or a VH sequence least 90%, 91%, 92%, 93%, 94%, 95%, 96%, 97%, 98%, 99% identical thereto, and the VL sequence of SEQ ID NO: 112, or a VL sequence at least 90%, 91%, 92%, 93%, 94%, 95%, 96%, 97%, 98%, 99% identical thereto; (3) the VH sequence of SEQ ID NO: 113, or a VH sequence least 90%, 91%, 92%, 93%, 94%, 95%, 96%, 97%, 98%, 99% identical thereto, and the VL sequence of SEQ ID NO: 112, or a VL sequence at least 90%, 91%, 92%, 93%, 94%, 95%, 96%, 97%, 98%, 99% identical thereto; (4) the VH sequence of SEQ ID NO: 96, or a VH sequence least 90%, 91%, 92%, 93%, 94%, 95%, 96%, 97%, 98%, 99% identical thereto, and the VL sequence of SEQ ID NO: 101, or a VL sequence at least 90%, 91%, 92%, 93%, 94%, 95%, 96%, 97%, 98%, 99% identical thereto; (5) the VH sequence of SEQ ID NO: 96, or a VH sequence least 90%, 91%, 92%, 93%, 94%, 95%, 96%, 97%, 98%, 99% identical thereto, and the VL sequence of SEQ ID NO: 109, or a VL sequence at least 90%, 91%, 92%, 93%, 94%, 95%, 96%, 97%, 98%, 99% identical thereto.

In some embodiments, the humanized antibody comprises: the VH framework region sequences VH FR1, VH FR2, VH FR3 and VH FR4 of any one antibody in Tables B and D,

In some embodiments, the VH framework region sequences VH FR1, VH FR2, VH FR3 and VH FR4 sequences comprise (i) amino acid sequences substantially identical (e.g., having at least about 80%, 85%, 90%, 92%, 95%, 97%, 98%, or 99% sequence identity) or identical to SEQ ID NOs: 84, 85, 86 and 87, respectively; (ii) amino acid sequences substantially identical (e.g., having at least about 80%, 85%, 90%, 92%, 95%, 97%, 98%, or 99% sequence identity) or identical to SEQ ID NOs: 89, 90, 91 and 87, respectively; (iii) amino acid sequences substantially identical (e.g., having at least about 80%, 85%, 90%, 92%, 95%, 97%, 98%, or 99% sequence identity) or identical to SEQ ID NOs: 93, 94, 95 and 87, respectively; (iv) amino acid sequences substantially identical (e.g., having at least about 80%, 85%, 90%, 92%, 95%, 97%, 98%, or 99% sequence identity) or identical to that of SEQ ID NOs: 132, 85, 133 and 87, respectively; (v) amino acid sequences substantially identical (e.g., having at least about 80%, 85%, 90%, 92%, 95%, 97%, 98%, or 99% sequence identity) or identical to that of SEQ ID NOs: 93, 126, 127 and 87, respectively; (vi) amino acid sequences substantially identical (e.g., having at least about 80%, 85%, 90%, 92%, 95%, 97%, 98%, or 99% sequence identity) or identical to that of SEQ ID NOs: 132, 133, 134 and 87, respectively; (vii) amino acid sequences substantially identical (e.g., having at least about 80%, 85%, 90%, 92%, 95%, 97%, 98%, or 99% sequence identity) or identical to that of SEQ ID NOs: 138, 94, 139 and 87, respectively; (viii) amino acid sequences substantially identical (e.g., having at least about 80%, 85%, 90%, 92%, 95%, 97%, 98%, or 99% sequence identity) or identical to that of SEQ ID NOs: 141, 142, 143 and 87, respectively.

In some embodiments, the humanized antibody comprises: the VL framework region sequences VL FR1, VL FR2, VL FR3 and VL FR4 of any one antibody in Tables C and D,

In some embodiments, the VL FR1, VL FR2, VL FR3 and VL FR4 sequences comprise (i) amino acid sequences substantially identical (e.g., having at least about 80%, 85%, 90%, 92%, 95%, 97%, 98%, or 99% sequence identity) or identical to SEQ ID NOs: 97, 98, 99 and 100, respectively; (ii) amino acid sequences substantially identical (e.g., having at least about 80%, 85%, 90%, 92%, 95%, 97%, 98%, or 99% sequence identity) or identical to SEQ ID NOs: 97, 102, 99 and 100, respectively; (iii) amino acid sequences substantially identical (e.g., having at least about 80%, 85%, 90%, 92%, 95%, 97%, 98%, or 99% sequence identity) or identical to SEQ ID NOs: 103, 104, 105 and 100, respectively; (iv) amino acid sequences substantially identical (e.g., having at least about 80%, 85%, 90%, 92%, 95%, 97%, 98%, or 99% sequence identity) or identical to SEQ ID NOs: 103, 107, 108 and 100, respectively; (v) amino acid sequences substantially identical (e.g., having at least about 80%, 85%, 90%, 92%, 95%, 97%, 98%, or 99% sequence identity) or identical to that of SEQ ID NOs: 134, 135, 136 and 131, respectively; (vi) amino acid sequences substantially identical (e.g., having at least about 80%, 85%, 90%, 92%, 95%, 97%, 98%, or 99% sequence identity) or identical to that of SEQ ID NOs: 128, 129, 130 and 131, respectively; (vii) amino acid sequences substantially identical (e.g., having at least about 80%, 85%, 90%, 92%, 95%, 97%, 98%, or 99% sequence identity) or identical to that of SEQ ID NOs: 145, 146, 147 and 131, respectively; (viii) amino acid sequences substantially identical (e.g., having at least about 80%, 85%, 90%, 92%, 95%, 97%, 98%, or 99% sequence identity) or identical to that of SEQ ID NOs: 97, 98, 152 and 100, respectively; or (ix) amino acid sequences substantially identical (e.g., having at least about 80%, 85%, 90%, 92%, 95%, 97%, 98%, or 99% sequence identity) or identical to that of SEQ ID NOs: 154, 102, 99 and 47, respectively.

In some embodiments. the VH FR1, VH FR2, VH FR3 and VH FR4 sequences comprise (i) SEQ ID NOs: 93, 94, 95 and 87, respectively, (ii) SEQ ID NOs: 132, 85, 133 and 87, respectively, (iii) SEQ ID NOs: 93, 126, 127 and 87, respectively, or (iv) SEQ ID NOs: 132, 133, 134 and 87, respectively; and/or the VL FR1, VL FR2, VL FR3 and VL FR4 sequences comprise (i) SEQ ID NOs: 134, 135, 136 and 131, respectively, or (ii) SEQ ID NOs: 128, 129, 130 and 131, respectively.

In some embodiments, the VH sequence comprises the amino acid sequence of SEQ ID NO: 96 and the VL sequence comprises the amino acid sequence of SEQ ID NO: 112, or the VH sequence comprises the amino acid sequence of SEQ ID NO: 113 and the VL sequence comprises the amino acid sequence of SEQ ID NO: 112.

In some embodiment, the VH sequence comprises the amino acid sequence of SEQ ID NO: 56 and VL sequence comprises the amino acid sequence of SEQ ID NO: 111.

In certain embodiments, the isolated monoclonal antibody or antigen-binding fragment thereof is afucosylated (e.g., absent of core fucose on the Fc N-glycan thus exhibiting increase IgG1 Fc binding affinity to FcγRIIIa), and comprises a VH sequence comprising the VH CDR1-CDR3 amino acid sequences of SEQ ID NOs: 114-116, and a VL sequence comprising the VL CDR1-CDR3 amino acid sequences of SEQ ID NOs: 120-122.

In certain embodiments, the isolated monoclonal antibody or antigen-binding fragment thereof is afucosylated (e.g., absent of core fucose on the Fc N-glycan thus exhibiting increase IgG1 Fc binding affinity to FcγRIIIa), and comprises a VH sequence comprising the amino acid sequence of SEQ ID NO: 113 and a VL sequence comprising the amino acid sequence of SEQ ID NO: 112.

In certain embodiments, the isolated monoclonal antibody or antigen-binding fragment thereof is afucosylated (e.g., absent of core fucose on the Fc N-glycan thus exhibiting increase IgG1 Fc binding affinity to FcγRIIIa), and comprises a VH sequence comprising the VH CDR1-CDR3 amino acid sequences of that of HFB2-4hz42hG1, and a VL sequence comprising the VL CDR1-CDR3 amino acid sequences of that of HFB2-4hz42hG1.

In certain embodiments, the isolated monoclonal antibody or antigen-binding fragment thereof is afucosylated (e.g., absent of core fucose on the Fc N-glycan thus exhibiting increase IgG1 Fc binding affinity to FcγRIIIa), and comprises a VH sequence comprising the amino acid sequence of the VH sequence of HFB2-4hz42hG1 and a VL sequence comprising the amino acid sequence of the VL sequence of HFB2-4hz42hG1.

In certain embodiments, the isolated monoclonal antibody or antigen-binding fragment thereof of the invention comprises a modified Fc region to enhance ADCC.

In certain embodiments, the modified Fc region comprises: (1) F243L/R292P/Y300L/V305I/P396L mutations to enhance FcγRIIIa binding; (2) S239D/I332E mutations to enhance FcγRIIIa binding; (3) S239D/I332E/A330L mutations to simultaneously enhance FcγRIIIa binding and decrease FcγRIIIb binding; (4) S298A/E333A/K334A mutations to enhance FcγRIIIa binding; and/or (5) afucosylated N297 at Fc region to enhance FcγRIIIa binding.

In certain embodiments, the antigen-binding fragment thereof is an Fab, Fab′, F(ab′)₂, F_{d}, single chain Fv or scFv, disulfide linked Fᵥ, V-NAR domain, IgNar, intrabody, IgGΔCH₂, minibody, F(ab′)₃, tetrabody, triabody, diabody, single-domain antibody, DVD-Ig, Fcab, mAb₂, (scFv)₂, or scFv-Fc.

In certain embodiments, the isolated monoclonal antibody or antigen-binding fragment thereof of the invention has a low (e.g., 1-5 or 1-2) pM range EC50 value for ADCC activity.

In certain embodiments, the isolated monoclonal antibody or antigen-binding fragment thereof of the invention has ADCC activity against primary B cells expressing surface hCXCR5, and/or primary T cells expressing surface hCXCR5.

In certain embodiments, the isolated monoclonal antibody or antigen-binding fragment thereof of the invention does not (or at most minimally) internalize the hCXCR5 surface antigen.

In certain embodiments, the isolated monoclonal antibody or antigen-binding fragment thereof of the invention inhibits cAMP signaling (e.g., EC50 less than 1 nM).

In certain embodiments, the isolated monoclonal antibody or antigen-binding fragment thereof of the invention inhibits chemotaxis (e.g., with ˜100% inhibition at about 0.1-0.5 nM, or about 0.1 nM).

In certain embodiments, the isolated monoclonal antibody or antigen-binding fragment thereof of the invention inhibits hCXCL13-induced B cell migration.

In certain embodiments, the isolated monoclonal antibody or antigen-binding fragment thereof of the invention does not substantially cross-react with hCXCR3.

In certain embodiments, the isolated monoclonal antibody or antigen-binding fragment thereof of the invention binds to hCXCR5 expressed on adherent cell lines (such as DX002) and/or suspension cell lines (such as M300-19).

In certain embodiments, the isolated monoclonal antibody or antigen-binding fragment thereof of the invention does not or minimally cross-reacts with cynomolgus monkey or mouse orthologs of hCXCR5.

In certain embodiments, the isolated monoclonal antibody or antigen-binding fragment thereof of the invention reduces the percentage of memory B cell population in a subject.

In certain embodiments, the isolated monoclonal antibody or antigen-binding fragment thereof of the invention binds hCXCR5 with a K_{d} of less than about 25 nM, 20 nM, 15 nM, 10 nM, 5 nM, 2 nM, or 1 nM or less.

Another aspect of the invention provides an isolated monoclonal antibody or an antigen-binding fragment thereof, which competes with the isolated monoclonal antibody or antigen-binding fragment thereof of any of the preceding embodiments for binding to the same epitope.

Another aspect of the invention provides a method of treating Sjögren syndrome (SS) in a subject in need thereof, the method comprising administering a therapeutically effective amount of an antibody of the invention (e.g., a humanized antibody of the invention) to the subject.

In certain embodiments, the method alleviates at least one symptom of SS.

Another aspect of the invention provides a method of treating lymphoma or leukemia in a subject in need thereof, the method comprising administering a therapeutically effective amount of an antibody of the invention (e.g., a humanized antibody of the invention) to the subject.

In certain embodiments, the lymphoma or leukemia is B cell lymphoma.

In certain embodiments, the B cell lymphoma is CLL (B-cell Chronic Lymphocytic Leukemia).

In certain embodiments, the lymphoma or leukemia is non-Hodgkin's lymphoma, such as Burkitt's lymphoma.

Another aspect of the invention provides a method of treating a disease or indication with ectopic germinal centers, including autoimmune disease or disorder, in a subject in need thereof, the method comprising administering a therapeutically effective amount of an antibody of the invention (e.g., a humanized antibody of the invention) to the subject.

In certain embodiments, the disease or indication is Rheumatoid Arthritis (RA), systemic lupus erythematosus (SLE), Celiac disease, Crohn's disease, ulcerative colitis, type I diabetes, multiple sclerosis (MS), Sarcoidosis, Psoriasis, Myasthenia gravis, Hashimoto's thyroiditis, Grave's disease, artherosclerosis, conjunctivitis, gastritis, hepatitis, or dermatitis.

Another aspect of the invention provides a method of treating solid cancer in a subject in need thereof, the method comprising administering a therapeutically effective amount of an antibody of the invention (e.g., a humanized antibody of the invention) to the subject, wherein the solid cancer is optionally gastric cancer, breast cancer, intestinal cancer, lung cancer, or prostate cancer.

In certain embodiments, the method of treating lymphoma or leukemia, and/or the method of treating solid cancer further comprising administering to the patient a chemotherapeutic agent, an anti-angiogenesis agent, a growth inhibitory agent, an immune-oncology agent, and/or an anti-neoplastic composition.

Another aspect of the invention provides polynucleotide encoding the heavy chain or the light chain or the antigen-binding portion thereof of the invention.

In certain embodiments, the polynucleotide is codon optimized for expression in a human cell.

Another aspect of the invention provides a vector comprising the polynucleotide of the invention.

In certain embodiments, the vector is an expression vector (e.g., a mammalian expression vector, a yeast expression vector, an insect expression vector, or a bacterial expression vector).

It should be understood that any one embodiment of the invention, including embodiments described only in the examples or claims, can be combined with any one or more additional embodiments of the invention, unless expressly disclaimed or unless is improper.

### BRIEF DESCRIPTION OF THE DRAWINGS

FIG. 1 shows sequence alignments for human CXCR5 and its closest paralog CXCR3, with a low 38.5% sequence identity and 51.5% sequence similarity.

FIGS. 2A and 2B show IMGT sequence alignments of the VH (FIG. 2A) and VL (FIG. 2B) regions of the various identified mouse anti-CXCR5 monoclonal antibodies. CDR1-CDR3 of the VH and VL sequences are highlighted. All antibodies are obtained from different VDJ recombination events (different families). Although HFB2-3 has a similar VH sequence to VH4 and VH5, the CDR3 are more divergent, thus conferring unique properties. HFB2-4 & HFB2-5 differs in 1 amino acid (a.a) in the CDR3 region sequence.

FIG. 3 shows the pharmacokinetic (PK) profile of chimeric anti-CXCR5 mouse monoclonal antibodies HFB2-4hG1 in wild-type mouse.

FIGS. 4A and 4B show IMGT sequence alignments of 3 humanized VH regions and 4 humanized VL regions, respectively, based on the mouse monoclonal antibody HFB2-4.

FIG. 5 shows sub-nM EC50 binding capacity of several chimeric monoclonal antibodies of the invention towards the hCXCR5 antigen expressed on adherent cell line.

FIG. 6A shows that the chimeric antibodies of the invention have essentially no cross-reactivity to cynomolgus and murine CXCR5 expressed on transiently transfected cells, based on antibody cross-reactivity assessment.

FIG. 6B shows that the chimeric antibodies of the invention do not bind to the closest ortholog of hCXCR5-hCXCR3.

FIG. 7 shows that the anti-CXCR5 antibodies of the invention efficiently inhibited ligand (CXCL13)-induced B-cell migration.

FIG. 8 shows effect of certain anti-CXCR5 monoclonal antibodies on intracellular cAMP signaling. The data showed that two of the chimeric antibodies efficiently blocked cAMP signaling upon ligand (CXCL13) activation.

FIG. 9A shows the results of ADCC reporter bioassay on six chimeric monoclonal antibodies of the invention. The data showed that the subject anti-CXCR5 monoclonal antibodies can trigger ADCC through engagement of CD16, with HFB2-4 demonstrating the most potent CD16 engagement.

FIG. 9B shows ADCC reporter bioassay using HFB2-4hG1 and HFB2-4hG1DE antibodies. An anti-CD20 IgG1 (positive control) and an isotype matched negative control IgG1 antibody were also included in the assay. Representative EC50 values (not necessarily matching those in the graph) are provided in the table below the graph.

FIG. 9C shows HFB2-4hG1DE-mediated ADCC lysis of Raji cells by primary NK cells, compared to positive control Rituximab and isotype control (MGO53-hG1DE).

FIG. 9D shows HFB2-4hG1DE-mediated ADCC lysis of primary B cells by primary NK cells, compared to positive control Rituximab and isotype control (MGO53-hG1DE).

FIG. 9E shows ADCC-mediated lysis of primary T cells by primary NK cells induced by HFB2-4hG1DE, compared to isotype control (MGO53-hG1DE) and no antibody (CD4⁺ cells+NK). Also see FIG. 9F.

FIG. 9F shows ADCC-mediated lysis of primary T cells by primary NK cells induced by afucosylated antibody AfuHFB2-4hG1, compared to Rituximab (hG1), isotype control (MGO53-hG1) and no antibody (CD4⁺ cells+NK) at E:T ratio of 5:1. Rituximab targets CD20 expressed on the surface of all B-cells, and thus Rituximab is not expected to target T cells that do not express CD20.

FIGS. 9G and 9H show results of ADCC reporter assay using HFB2-4hG1DE, rituximab in hG1 format (positive control) and DE isotype control at E:T ratio of 3:1 with B cells from primary Sjögren syndrome (SS) patients (FIG. 9G) and lymphoproliferative pSS patients (FIG. 9H) as target cells. Top graphs: luminescence readout of the ADCC reporter assay (RLU). Bottom graphs: fold induction, calculated as RLU (with antibody−background)/RLU (cells alone−background). In the bottom graphs, left bars: HFB2-4hG1DE, middle bars: positive control, right bars: isotype control.

FIGS. 10A and 10B show measurement of antibody internalization by two different protocols. The results showed that neither HFB2-4hz9-hG1DE nor HFB2-4hz12-hG1DE (both humanized antibodies based on the HFB2-4 chimeric antibody) internalize as compared to the positive control (CD71) antibody.

FIGS. 11A and 11B show results of ADCC reporter bioassay using selected humanized variant antibodies.

FIG. 12 shows chemotaxis inhibition by selected humanized variants.

FIG. 13 shows ADCC reporter bioassay using primary B cells.

FIG. 14 shows target binding to different cancer cell lines.

FIG. 15 shows ADCC reporter assay on B cell lymphoma cell lines.

FIG. 16 shows the results of anti-tumor in vivo efficacy study of HFB2-4-hG1. Both HFB2-4hG1 and the positive control are statistically significant (p<0.00001****) compared to the controls (PBS or the isotype matched MGO53-hG1 antibody).

FIG. 17 shows ADCC activity of the subject antibody on Sjögren Patients' B cells.

FIG. 18 shows the reduction of the percentage of memory B cell population in pSS patient's samples by the subject antibody.

FIGS. 19A and 19B show binding of additional humanized variants of HFB2-4hG1 (HFB2-4 hz-hG1) to Raji cells. Arrows indicate humanized variants with the top binding profiles.

FIG. 20 shows pharmacokinetic profiles of top nine humanized HFB2-4 hz-hG1 variants

FIG. 21 shows binding of top 6 HFB2-4 hz-hG1 variants to CXCR5+ Raji cells (left panel) and ADCC activity to engage CD16 in reporter system.

FIG. 22 shows binding of HFB2-4 hz variants in hG1DE format to adherent DX002 cells expressing CXCR5.

FIG. 23 shows pharmacokinetic profiles of top four humanized HFB2-4 hz in parental hG1 format (left panel) and hG1DE format (right panel).

FIG. 24A shows binding of afucosylated HFB2-4hz42-hG1 (afu-HFB2-4hz42-hG1), afucosylated parent HFB2-4hG1 (afu-HFB2-4hG1), afucosylated benchmark hG1 antibody and isotype control (MGO53-hG1) to DX002 cells expressing CXCR5.

FIG. 24B shows CD16 engagement determined by ADCC reporter assay of afu-HFB2-4hz42-hG1, afu-HFB2-4hG1, afucosylated benchmark hG1 antibody and isotype control (MGO53-hG1).

FIG. 25 shows ADCC-mediated lysis of primary B cells from healthy donors by primary NK cells mediated by afucosylated HFB2-4hz42-hG1 (afu-HFB2-4hz42-hG1) compared to benchmark and isotype control (MGO53-hG1) antibody.

FIG. 26 shows ADCC-mediated lysis of B cells from SS patients by primary NK cells mediated by afucosylated HFB2-4hz42-hG1 (afu-HFB2-4hz42-hG1) compared to benchmark and isotype control (MGO53-hG1) antibody.

FIGS. 27A and 27B show results of testing for complement-dependent cytotoxicity (CDC) activity of afucosylated HFB2-4hz42-hG1 (afu-HFB2-4hz42-hG1), afucosylated parent HFB2-4hG1 (afu-HFB2-4hG1), rituximab in hG1 format (positive control) and isotype control MGO53-hG1. Serum was used to provide the complement system. In FIG. 27B, two different sera were tested.

FIG. 28A shows pharmacokinetic profiles of afucosylated HFB2-4hz42-hG1 (afu-HFB2-4hz42-hG1), afucosylated parent HFB2-4hG1 (afu-HFB2-4hG1) and benchmark antibody in wild-type mice (n=1).

FIG. 28B shows pharmacokinetic profiles of afucosylated HFB2-4hz42-hG1 (afu-HFB2-4hz42-hG1) in 1 male and 2 female cynomolgous monkey.

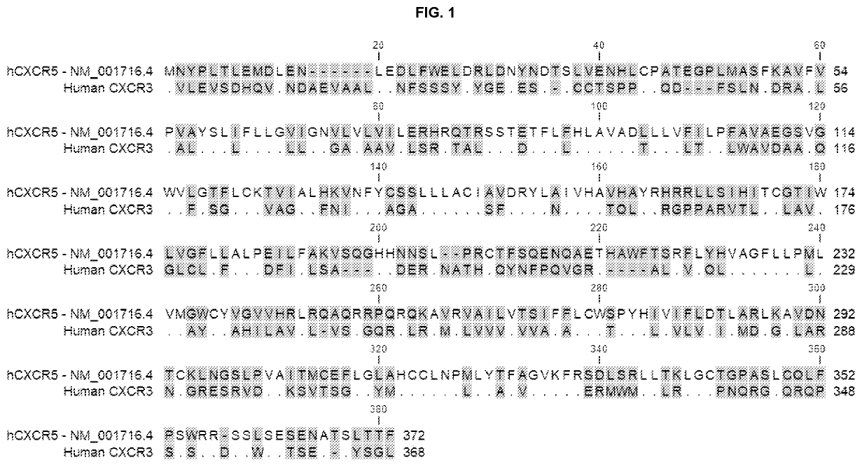

### DETAILED DESCRIPTION OF THE INVENTION

### 1. Overview

According to the invention described herein, mice were immunized with recombinant human CXCR5 using multiple immunization strategies. Combined with Applicant's proprietary single-cell based antibody screening technology, six high affinity anti-CXCR5 mouse monoclonal antibodies, HFB2-1 to HBF2-6, were identified from three independent screens.

These antibodies were further tested for desired functional characteristics, including specificity (e.g., little cross-reactivity to hCXCR3), binding affinity to CXCR5 expressed on cell lines (e.g., adherent cell lines such as DX002 and suspension cell lines such as M300-19), cross-reactivity (e.g., cynomolgus monkey or mouse orthologs of hCXCR5), effects on B cell migration (chemotaxis), effects on intracellular cAMP levels, ADCC reporter assay, internalization assay using CXCR5-expressing stable cell lines, and PK profile in mouse, etc.

For example, FACS (flow cytometry) analysis showed that all except one leading candidate antibodies showed sub-nM EC50 values against hCXCR5-expressing CHO cells, and none of the lead antibodies cross-reacted with cynomolgus or mouse CXCR5 orthologs. In addition, none of the lead antibodies bind to the hCXCR3 paralog, confirming their specificity. The lead antibodies also efficiently blocked hCXCL13-induced B cell migration, and the HFB2-4 chimeric antibody is the most efficient to inhibit chemotaxis. HFB2-4 & HFB2-5 efficiently blocked hCXCL13-induced cAMP signaling, and are superior to the benchmark antibody. ADCC reporter assay (Promega) showed that the anti-hCXCR5 mAbs (especially HFB2-4) engaged CD16.

Based on these test, among the 6 single-digit nanomolar affinity antibodies, HFB2-4 was chosen as the one with the highest potency and maximal effect in the functional assays, and was further analyzed for its PK profile in wild-type mice. The results showed that HFB2-4 demonstrated a favorable PK profile in mice.

Preliminary results of testing the anti-tumor efficacy of HFB2-4 in a mouse Raji model of tumor also showed promising anti-tumor effect, which is on par with that of Rituximab as a positive control.

Based on its overall superior biological profile, HFB2-4 was selected as the lead antibody for further testing and for humanization.

At least 12 humanized variants (all having the S239D/I332E mutations) have been generated by combining 3 VH and 4 VL region CDR sequences (VH1 with each of VL1-VL4, VH2 with each of VL1-VL4, and VH3 with each of VL1-VL4), and characterized; and an additional 13 variants were further synthesized. Of note, all the humanized (Hz) variants have a first match of a human germline sequence according to the IMGT system. The majority of these humanized variants preserved their physico-chemical (affinity to the target, stability, solubility, etc.) and/or biological activity (blockage or stimulation of the target, ADCC, etc.).

These humanized variant antibodies were further tested for desired functional characteristics, including specificity (e.g., little cross-reactivity to hCXCR3), binding affinity to CXCR5 expressed on cell lines (e.g., adherent cell lines such as DX002 and suspension cell lines such as M300-19), cross-reactivity (e.g., cynomolgus monkey or mouse orthologs of hCXCR5), effects on B cell migration (chemotaxis), effects on intracellular cAMP levels, internalization assay using CXCR5-expressing stable cell lines, antibody developability assessments (such as SEC and SDS-PAGE analysis), ADCC reporter assay, and PK profile in mouse, etc.

The results showed that 10 out of 12 HFB2-4 hz variants showed comparable binding to hCXCR5 as the parental antibody HFB2-4; blocking of cAMP signaling by 9 out of 12 HFB2-4 hz variants; potent chemotaxis inhibition by the Hz variants, with HFB2-4hz12 being the most potent (˜100% at 0.1 nM), and a comparable effect in HFB2-4hz9; and efficient blocking of hCXCL13-induced B cell migration.

Based on its overall superior biological profile, HFB2-4hz12 was selected as the lead candidate for ADCC testing. The result showed that HFB2-4hz12hG1DE, as well as HFB2-4hz9hG1DE, have potent ADCC activity reaching low pM range (1-2 pM) EC50. The data further showed that the humanization process did not modify the high ADCC potency of the candidate antibody (e.g., the HFB2-4DE antibody). Further, HFB2-4hz12hG1DE showed ADCC activity on primary B cells expressing hCXCR5.

Internalization studies showed that the HFB2-4hz12-hG1DE and HFB2-4hz9-hG1DE Hz variants do not (or at most minimally) internalize the hCXCR5 surface antigen.

PK profiling showed that HFB2-4hz12-hG1DE has a shorter half-life in plasma (4.5h) than the parent antibody HFB2-4hG1 (109 h).

Selected Hz antibodies including HFB2-4hz2hG1, HFB2-4hz9hG1DE, HFB2-4hz10hG1DE, HFB2-4hz11hG1DE, and HFB2-4hz12hG1DE were also tested for a number of developability assays, including accelerated stability studies at 25 and 40° C. for up to 14 days, forced degradation studies at 25° C. in 100 mM acetate, pH 3.5 for up to 6 hours; and up to three freeze/thaw cycles in PBS, pH 7.4 (all 2 mg/mL antibody concentration). The results showed that HFB2-4hz12hG1DE has the most stable profile by SDS-PAGE gels. Overall, HFB2-4hz9hG1DE and HFB2-4hz12hG1DE showed the most favorable profiles of the tested antibodies, and were chosen for generating further Hz variants based thereon.

Numerous additional second round humanization antibodies were generated and selected. Such second round humanization (Hz) variants are listed in the table below. All such humanized variants tested bind hCXCR5 with sub-nM EC50 values. All such Hz variants bind hCXCR5 with sub-nM EC50s. Comparable binding properties were found for parental antibodies and humanized variant antibodies, including binding affinity to hCXCR5, and engagement of CD16 to induce ADCC.

TABLE 1
Selected Second Round Humanization Variants

| | | | | | Exp 1 | Exp 2 |
|---|---|---|---|---|---|---|
| | V-gene Name | % Humanization | V-gene Name | | EC50 | EC50 |
| Ab Variants | (VH) | (VH) | (VL) | % Humanization(VL) | (nM) | (nM) |
| HFB2-4hG1DE | Chimeric parental | | | | 1 | 1.3 |
| HFB2-4hz9-hG1DE | VH3 | 87.4 | VL1 | 91.4 | 1.3 | 1 |
| HFB2-4hz14-hG1DE | VH3 | 87.4 | VL1b | 96.8 | 0.4 | 0.1 |
| HFB2-4hz15-hG1DE | VH3 | 87.4 | VL1c | 88.2 | 0.6 | 0.05 |
| HFB2-4hz18-hG1DE | VH3c | 83.2 | VL1 | 91.4 | 0.6 | 0.1 |
| HFB2-4hz19-hG1DE | VH3d | 86.3 | VL1 | 91.4 | 0.5 | 0.2 |
| HFB2-4hz12-hG1DE | VH3 | 87.4 | VL4 | 83.9 | 1.2 | 0.3 |
| HFB2-4hz24-hG1DE | VH3 | 87.4 | VL4c | 79.6 | 0.5 | 0.2 |
| HFB2-4hz27-hG1DE | VH3c | 83.2 | VL4 | 83.9 | 0.3 | 0.8 |
| HFB2-4hz28-hG1DE | VH3d | 86.3 | VL4 | 83.9 | 0.7 | 1.8 |
| HFB2-4hz31-hG1DE | VH3c | 83.2 | VL1b | 96.8 | 0.4 | 0.4 |
| HFB2-4hz32-hG1DE | VH3c | 83.2 | VL1c | 88.2 | 0.5 | 0.5 |
| HFB2-4hz33-hG1DE | VH3d | 86.3 | VL1b | 96.8 | 0.3 | 0.3 |
| HFB2-4hz34-hG1DE | VH3d | 86.3 | VL1c | 88.2 | 0.6 | 0.5 |
| HFB2-4hz35-hG1DE | VH3c | 83.2 | VL4c | 79.6 | 0.7 | 0.6 |
| HFB2-4hz36-hG1DE | VH3d | 86.3 | VL4c | 79.6 | 0.5 | 0.4 |

Among these secondary Hz variants, HFB2-4hz14-hG1DE and HFB2-4hz15-hG1DE were selected for further developability assessment (see assays described above). The results showed that the Hz14 and Hz15 DE variants do not show instabilities at 25 and 40° C. when formulated in 20 mM acetate, pH 6.0. In addition, stability is generally retained after freeze/thaw cycles and low pH stress treatment (pH3.5 up to 6 hours). Overall, the humanized variants of HFB2-4, including HFB2-4hz9-hG1DE, HFB2-4hz12-hG1DE, HFB2-4hz14-hG1DE, and HFB2-4hz15-hG1DE, all exhibit favorable developability profiles. One of these leading candidates—HFB2-4hz12-hG1DE—was selected for further development.

Further humanization variants based on HFB2-4 were generated based on HFB2-4hG1 (HFB2-4 hz-hG1), including HFB2-4hz37-hG1 and HFB2-4hz42-hG1. See Example 5 and especially Table 4.

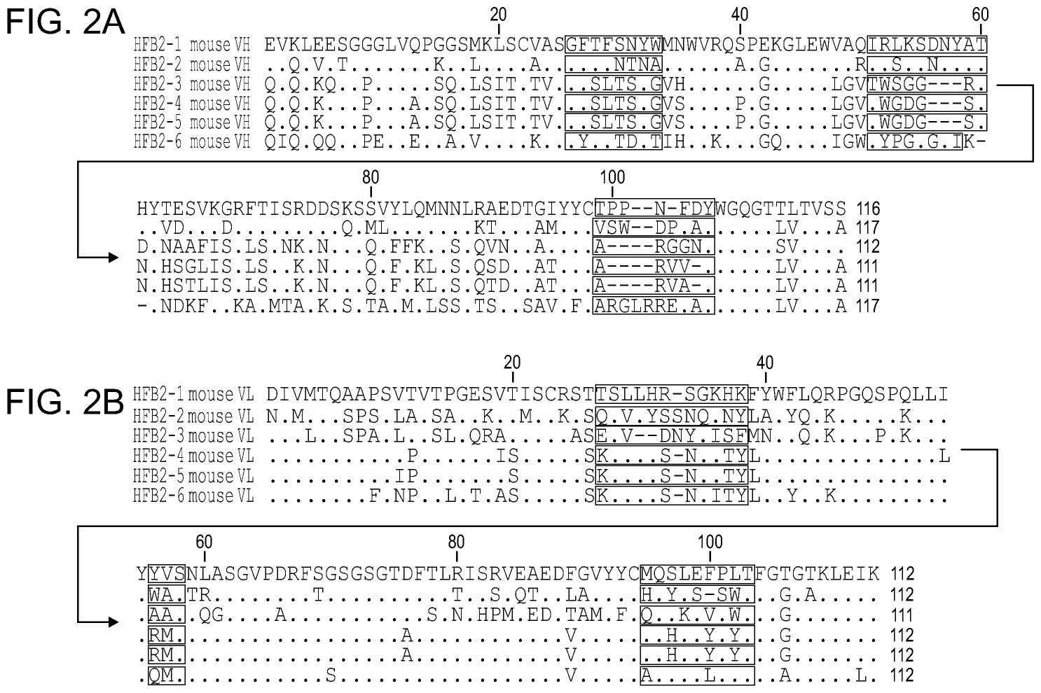

Thus the invention described herein provides an isolated monoclonal antibody, or an antigen-binding fragment thereof, wherein said monoclonal antibody or antigen-binding fragment thereof is specific for human CXCR5 (hCXCR5), and wherein said monoclonal antibody comprises: (1) a heavy chain variable region (HCVR), comprising a HCVR CDR1 sequence, a HCVR CDR2 sequence, and a HCVR CDR3 sequence of any one of the HCVR CDR1, CDR2, and CDR3 sequences, respectively, of the monoclonal antibodies in FIG. 2A (e.g., HFB2-1, HFB2-2, HFB2-3, HFB2-4, HFB2-5, and HFB2-6); and (2) a light chain variable region (LCVR), comprising a LCVR CDR1 sequence, a LCVR CDR2 sequence, and a LCVR CDR3 sequence of any one of the LCVR CDR1, CDR2, and CDR3 sequences, respectively, of the monoclonal antibodies in FIG. 2B (e.g., HFB2-1, HFB2-2, HFB2-3, HFB2-4, HFB2-5, and HFB2-6).

In certain embodiments, the antibody of the invention comprises (1) the HCVR CDR1 sequence, the HCVR CDR2 sequence, and the HCVR CDR3 sequence of HFB2-4 in FIG. 2A; and (2) the (corresponding) LCVR CDR1 sequence, the LCVR CDR2 sequence, and the LCVR CDR3 sequence of HFB2-4 in FIG. 2B.

In certain embodiments, the antibody of the invention is a mouse-human chimeric antibody comprising constant region sequences of a human antibody (such as hIgG1, or hIgG2), and the HCVR of any one of the HCVR sequences of the monoclonal antibodies in FIG. 2A (e.g., HFB2-1, HFB2-2, HFB2-3, HFB2-4, HFB2-5, and HFB2-6); and the LCVR of any one of the (corresponding) LCVR sequences of the monoclonal antibodies in FIG. 2B (e.g., HFB2-1, HFB2-2, HFB2-3, HFB2-4, HFB2-5, and HFB2-6).

In certain embodiments, the mouse-human chimeric antibody comprises the HCVR sequence of HFB2-4 in FIG. 2A, and the LCVR sequence of HFB2-4 in FIG. 2B.

In certain embodiments, the antibody of the invention is a humanized antibody.

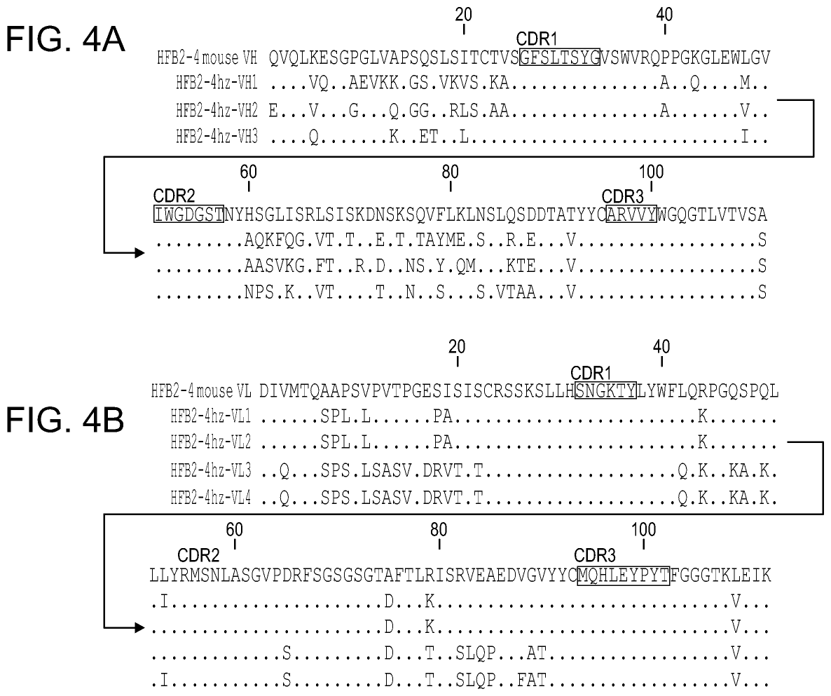

In certain embodiments, the humanized antibody comprises: (1) the HCVR CDR1 sequence, the HCVR CDR2 sequence, and the HCVR CDR3 sequence of the monoclonal antibodies in FIG. 4A; and (2) the LCVR CDR1 sequence, the LCVR CDR2 sequence, and the LCVR CDR3 sequence of the monoclonal antibodies in FIG. 4B.

In certain embodiments, the humanized antibody comprises: (1) the framework region sequence of any one of the HCVR sequences of the VH1, VH2, and VH3 sequences in FIG. 4A, and/or (2) the framework region sequence of any one of the LCVR sequences of the VL1, VL2, VL3, and VL4 sequences in FIG. 4B.

In certain embodiments, the humanized antibody comprises: (1) the framework region sequence of VH3 in FIG. 4A, and/or (2) the framework region sequence of VL1 in FIG. 4B.

In certain embodiments, the humanized antibody comprises: (1) the framework region sequence of VH3 in FIG. 4A, and/or (2) the framework region sequence of VL4 in FIG. 4B.

In certain embodiments, the antibody of the invention has a modified Fc region to enhance ADCC. For example, in certain embodiments, the antibody of the invention comprises F243L/R292P/Y300L/V305I/P396L mutations to enhance FcγRIIIa binding. In certain embodiments, the antibody of the invention comprises S239D/I332E mutations to enhance FcγRIIIa binding (hIgG1-DE herein). In certain embodiments, the antibody of the invention comprises S239D/I332E/A330L mutations to simultaneously enhance FcγRIIIa binding and decrease FcγRIIIb binding. In certain embodiments, the antibody of the invention comprises S298A/E333A/K334A mutations to enhance FcγRIIIa binding.

In certain embodiments, the antibody of the invention is a humanized mouse monoclonal antibody, optionally, the antibody comprises S239D/I332E mutations to enhance FcγRIIIa binding (hIgG1-DE). For example, the humanized monoclonal antibody may be produced by CDR grafting of the CDR regions of the mouse monoclonal antibody into a human framework region.

In certain embodiments, the antibody of the invention has low (e.g., 1-5 or 1-2) pM range EC50 value for ADCC activity.

In certain embodiments, the antibody of the invention has ADCC activity against primary B cells expressing surface hCXCR5.

In certain embodiments, the antibody of the invention does not (or at most minimally) internalize the hCXCR5 surface antigen.

In certain embodiments, the antibody of the invention inhibits cAMP signaling.

In certain embodiments, the antibody of the invention inhibits chemotaxis (e.g., with ˜100% inhibition at about 0.1-0.5 nM, or about 0.1 nM).

In certain embodiments, the antibody of the invention inhibits hCXCL13-induced B cell migration.

In certain embodiments, the antibody of the invention is specific for hCXCR5 (e.g., has no or little cross-reactivity to hCXCR3.

In certain embodiments, the antibody of the invention binds to hCXCR5 expressed on adherent cell lines (such as DX002) and/or suspension cell lines (such as M300-19).

In certain embodiments, the antibody of the invention does not or minimally cross-reacts with cynomolgus monkey or mouse orthologs of hCXCR5.

In certain embodiments, the antibody of the invention reduces the percentage of memory B cell population in a subject.

Another aspect of the invention provides a method of treating Sjögren syndrome (SS) in a subject in need thereof, the method comprising administering a therapeutically effective amount of an antibody of the invention (e.g., a humanized antibody of the invention) to the subject.

About 25% of the Sjögren syndrome patients show ectopic germinal centers (GC) with high infiltration of CXCR5⁺ cells into salivary glands, GCs in SS patients are rich in CXCL13-producing Tfh cells that recruit and differentiate CXCR5⁺ B cells into antibody-producing cells. CXCL13 levels have been found to be elevated in mouse SS models and in human disease. In addition, neutralization of CXCL13 has been found protective in animal models of Sjögren's disease.

In certain embodiments, the method alleviates at least one symptom of SS.

Another aspect of the invention provides a method of treating lymphoma or leukemia in a subject in need thereof, the method comprising administering a therapeutically effective amount of an antibody of the invention (e.g., a humanized antibody of the invention) to the subject.

In certain embodiments, the lymphoma or leukemia is B cell lymphoma.

In certain embodiments, the B cell lymphoma is CLL (B-cell Chronic Lymphocytic Leukemia).

In certain embodiments, the lymphoma or leukemia is non-Hodgkin's lymphoma, such as Burkitt's lymphoma.

Another aspect of the invention provides a method of treating a disease or indication with ectopic germinal centers, including autoimmune disease or disorder, in a subject in need thereof, the method comprising administering a therapeutically effective amount of an antibody of the invention (e.g., a humanized antibody of the invention) to the subject.

In certain embodiments, the disease or indication is Rheumatoid Arthritis (RA), systemic lupus erythematosus (SLE), Celiac disease, Crohn's disease, ulcerative colitis, type I diabetes, multiple sclerosis (MS), Sarcoidosis, Psoriasis, Myasthenia gravis, Hashimoto's thyroiditis, Grave's disease, artherosclerosis, conjunctivitis, gastritis, hepatitis, or dermatitis.

Another aspect of the invention provides a method of treating solid cancer in a subject in need thereof, the method comprising administering a therapeutically effective amount of an antibody of the invention (e.g., a humanized antibody of the invention) to the subject.

In certain embodiments, the solid cancer is gastric cancer, breast cancer, intestinal cancer, lung cancer, or prostate cancer.

Detailed aspects of the invention are described further and separately in the various sections below. However, it should be understood that any one embodiment of the invention, including embodiments described only in the examples or drawings, and embodiments described only under one section below, can be combined with any other embodiment(s) of the invention.

### 2. Definitions

The term “antibody,” in the broadest sense, encompasses various antibody structures, including but not limited to monoclonal antibodies, polyclonal antibodies, and multispecific antibodies (e.g., bispecific antibodies). The term “antibody” may also broadly refers to a molecule comprising complementarity determining region (CDR) 1, CDR2, and CDR3 of a heavy chain and CDR1, CDR2, and CDR3 of a light chain, wherein the molecule is capable of binding to an antigen. The term “antibody” also includes, but is not limited to, chimeric antibodies, humanized antibodies, human antibodies, and antibodies of various species such as mouse, human, cynomolgus monkey, etc.

In a narrower sense, however, “antibody” refers to the various monoclonal antibodies, including chimeric monoclonal antibodies, humanized monoclonal antibodies, and human monoclonal antibodies, particularly humanized monoclonal antibodies of the invention.

In some embodiments, an antibody comprises a heavy chain variable region (HCVR) and a light chain variable region (LCVR). In some embodiments, an antibody comprises at least one heavy chain (HC) comprising a heavy chain variable region and at least a portion of a heavy chain constant region, and at least one light chain (LC) comprising a light chain variable region and at least a portion of a light chain constant region. In some embodiments, an antibody comprises two heavy chains, wherein each heavy chain comprises a heavy chain variable region and at least a portion of a heavy chain constant region, and two light chains, wherein each light chain comprises a light chain variable region and at least a portion of a light chain constant region.

As used herein, a single-chain Fv (scFv), or any other antibody that comprises, for example, a single polypeptide chain comprising all six CDRs (three heavy chain CDRs and three light chain CDRs) is considered to have a heavy chain and a light chain. In some such embodiments, the heavy chain is the region of the antibody that comprises the three heavy chain CDRs and the light chain in the region of the antibody that comprises the three light chain CDRs.

The term “heavy chain variable region (HCVR)” as used herein refers to, at a minimum, a region comprising heavy chain CDR1 (CDR-H1), framework 2 (HFR2), CDR2 (CDR-H2), FR3 (HFR3), and CDR3 (CDR-H3). In some embodiments, a heavy chain variable region also comprises at least a portion (e.g., the whole) of an FR1 (HFR1), which is N-terminal to CDR-H1, and/or at least a portion (e.g., the whole) of an FR4 (HFR4), which is C-terminal to CDR-H3.

The term “heavy chain constant region” as used herein refers to a region comprising at least three heavy chain constant domains, CH1, CH2, and CH3. Non-limiting exemplary heavy chain constant regions include γ, δ, and α. Non-limiting exemplary heavy chain constant regions also include ε and μ. Each heavy constant region corresponds to an antibody isotype. For example, an antibody comprising a γ constant region is an IgG antibody, an antibody comprising a δ constant region is an IgD antibody, an antibody comprising an α constant region is an IgA antibody, an antibody comprising an ε constant region is an IgE antibody, and an antibody comprising an μ constant region is an IgM antibody.

Certain isotypes can be further subdivided into subclasses. For example, IgG antibodies include, but are not limited to, IgG1 (comprising a γ1 constant region), IgG2 (comprising a γ2 constant region), IgG3 (comprising a γ3 constant region), and IgG4 (comprising a γ4 constant region) antibodies; IgA antibodies include, but are not limited to, IgA1 (comprising an α1 constant region) and IgA2 (comprising an α2 constant region) antibodies; and IgM antibodies include, but are not limited to, IgM1 (comprising an μ1 constant region) and IgM2 (comprising an μ2 constant region).

The term “heavy chain” as used herein refers to a polypeptide comprising at least a heavy chain variable region, with or without a leader sequence. In some embodiments, a heavy chain comprises at least a portion of a heavy chain constant region. The term “full-length heavy chain” as used herein refers to a polypeptide comprising a heavy chain variable region and a heavy chain constant region, with or without a leader sequence, and with or without a C-terminal lysine.

The term “light chain variable region (LCVR)” as used herein refers to a region comprising light chain CDR1 (CDR-L1), framework (FR) 2 (LFR2), CDR2 (CDR-L2), FR3 (LFR3), and CDR3 (CDR-L3). In some embodiments, a light chain variable region also comprises at least a portion (e.g., the whole) of an FR1 (LFR1) and/or at least a portion (e.g., the whole) of an FR4 (LFR4).

The term “light chain constant region” as used herein refers to a region comprising a light chain constant domain, C_{L}. Non-limiting exemplary light chain constant regions include λ and κ.

The term “light chain” as used herein refers to a polypeptide comprising at least a light chain variable region, with or without a leader sequence. In some embodiments, a light chain comprises at least a portion of a light chain constant region. The term “full-length light chain” as used herein refers to a polypeptide comprising a light chain variable region and a light chain constant region, with or without a leader sequence.

The term “antibody fragment” or “antigen binding portion” (of antibody) includes, but is not limited to, fragments that are capable of binding antigen, such as Fv, single-chain Fv (scFv), Fab, Fab′, and (Fab′)₂. In certain embodiments, an antibody fragment includes Fab, Fab′, F(ab′)₂, F_{d}, single chain Fv or scFv, disulfide linked Fᵥ, V-NAR domain, IgNar, intrabody, IgGΔCH2, minibody, F(ab′)₃, tetrabody, triabody, diabody, single-domain antibody, DVD-Ig, Fcab, mAb₂, (scFv)₂, or scFv-Fc.

The term “Fab” refers to an antibody fragment with a molecular mass of approximately 50,000 Daltons, and has an activity of binding to the antigen. It comprises approximately half of the N-terminal side of the heavy chain and the whole of the light chain connected by a disulfide bridge. The Fab can be obtained in particular by treatment of immunoglobulin by a protease, papain.

The term “F(ab′)₂” designates a fragment of approximately 100,000 Daltons and an activity of binding to the antigen. This fragment is slightly larger than two Fab fragments connected via a disulfide bridge in the hinge region. These fragments are obtained by treating an immunoglobulin with a protease, pepsin. The Fab fragment can be obtained from the F(ab′)2 fragment by cleaving of the disulfide bridge of the hinge region.

A single Fv chain “scFv” corresponds to a VH: VL polypeptide synthesized using the genes coding for the VL and VH domains and a sequence coding for a peptide intended to bind these domains. An scFv according to the invention includes the CDRs maintained in an appropriate conformation, for example using genetic recombination techniques.

The dimers of “scFv” correspond to two scFv molecules connected together by a peptide bond. This Fv chain is frequently the result of the expression of a fusion gene including the genes coding for VH and VL connected by a linker sequence coding a peptide. The human scFv fragment may include CDR regions that are maintained in an appropriate conformation, preferably by means of the use of genetic recombination techniques.

The “dsFv” fragment is a VH-VL heterodimer stabilized by a disulfide bridge; it may be divalent (dsFV₂). Fragments of divalent Sc(Fv)₂ or multivalent antibodies may form spontaneously by the association of monovalent scFvs or be produced by connecting scFvs fragments by peptide binding sequences.

The Fc fragment is the support for the biological properties of the antibody, in particular its ability to be recognized by immunity effectors or to activate the complement. It consists of constant fragments of the heavy chains beyond the hinge region.

The term “diabodies” signifies small antibody fragments having two antigen fixing sites. These fragments comprise, in the same VH-VL polypeptide chain, a variable heavy chain domain VH connected to a variable light chain domain VL. Using a binding sequence that is too short to allow the matching of two domains of the same chain, the matching with two complementary domains of another chain necessarily occurs and thus two antigen fixing sites are created.

An “antibody that binds to the same epitope” as a reference antibody can be determined by an antibody competition assay. It refers to an antibody that blocks binding of the reference antibody to its antigen in a competition assay by 50% or more, and conversely, the reference antibody blocks binding of the antibody to its antigen in a competition assay by 50% or more. The term “compete” when used in the context of an antibody that compete for the same epitope means competition between antibodies is determined by an assay in which an antibody being tested prevents or inhibits specific binding of a reference antibody to a common antigen.

Numerous types of competitive binding assays can be used, for example: solid phase direct or indirect radioimmunoassay (RIA), solid phase direct or indirect enzyme immunoassay (EIA), sandwich competition assay (see, e.g., Stahli et al., 1983, Methods in Enzymology 9:242-253); solid phase direct biotin-avidin EIA (see, e.g., Kirkland et al., 1986, J. Immunol. 137:3614-3619); solid phase direct labeled assay; solid phase direct labeled sandwich assay (see, e.g., Harlow and Lane, 1988, Antibodies, A Laboratory Manual, Cold Spring Harbor Press); solid phase direct label RIA using I¹²⁵ label (see, e.g., Morel et al., 1988, Molec. Immunol. 25:7-15); solid phase direct biotin-avidin EIA (see, e.g., Cheung, et al., 1990, Virology 176:546-552); and direct labeled RIA (Moldenhauer et al., 1990, Scand. J. Immunol.).

Typically, such an assay involves the use of purified antigen bound to a solid surface or cells bearing either of these, an unlabeled test antigen binding protein and a labeled reference antibody. Competitive inhibition is measured by determining the amount of label bound to the solid surface or cells in the presence of the test antibody. Usually the test antibody is present in excess. Antibodies identified by competition assay (competing antibodies) include antibodies binding to the same epitope as the reference antibodies and antibodies binding to an adjacent epitope sufficiently proximal to the epitope bound by the reference antibody for steric hindrance to occur. In some embodiments, when a competing antibody is present in excess, it will inhibit specific binding of a reference antibody to a common antigen by at least 40%, 45%, 50%, 55%, 60%, 65%, 70% or 75%. In some instance, binding is inhibited by at least 80%, 85%, 90%, 95%, or 97% or more.

The term “antigen” refers to a molecule or a portion of a molecule capable of being bound by a selective binding agent, such as an antibody or immunologically functional fragment thereof, and additionally capable of being used in a mammal to produce antibodies capable of binding to that antigen. An antigen may possess one or more epitopes that are capable of interacting with antibodies.

The term “epitope” is the portion of an antigen molecule that is bound by a selective binding agent, such as an antibody or a fragment thereof. The term includes any determinant capable of specifically binding to an antibody. An epitope can be contiguous or non-contiguous (e.g., in a polypeptide, amino acid residues that are not contiguous to one another in the polypeptide sequence but that within in context of the molecule are bound by the antigen binding protein). In some embodiments, epitopes may be mimetic in that they comprise a three dimensional structure that is similar to an epitope used to generate the antibody, yet comprise none or only some of the amino acid residues found in that epitope used to generate the antibody. Epitope determinants may include chemically active surface groupings of molecules such as amino acids, sugar side chains, phosphoryl or sulfonyl groups, and may have specific three dimensional structural characteristics, and/or specific charge characteristics.

In some embodiments, an “epitope” is defined by the method used to determine it. For example, in some embodiments, an antibody binds to the same epitope as a reference antibody, if they bind to the same region of the antigen, as determined by hydrogen-deuterium exchange (HDX).

In certain embodiments, an antibody binds to the same epitope as a reference antibody if they bind to the same region of the antigen, as determined by X-ray crystallography.

A “chimeric antibody” as used herein refers to an antibody comprising at least one variable region from a first species (such as mouse, rat, cynomolgus monkey, etc.) and at least one constant region from a second species (such as human, cynomolgus monkey, chicken, etc.). In some embodiments, a chimeric antibody comprises at least one mouse variable region and at least one human constant region. In some embodiments, all of the variable regions of a chimeric antibody are from a first species and all of the constant regions of the chimeric antibody are from a second species.

A “humanized antibody” as used herein refers to an antibody in which at least one amino acid in a framework region of a non-human variable region (such as mouse, rat, cynomolgus monkey, chicken, etc.) has been replaced with the corresponding amino acid from a human variable region. In some embodiments, a humanized antibody comprises at least one human constant region or fragment thereof. In some embodiments, a humanized antibody fragment is an Fab, an scFv, a (Fab′)₂, etc.

A “CDR-grafted antibody” as used herein refers to a humanized antibody in which one or more complementarity determining regions (CDRs) of a first (non-human) species have been grafted onto the framework regions (FRs) of a second (human) species.

A “human antibody” as used herein refers to antibodies produced in humans, antibodies produced in non-human animals that comprise human immunoglobulin genes, such as XENOMOUSE®, and antibodies selected using in vitro methods, such as phage display, wherein the antibody repertoire is based on a human immunoglobulin sequences.

An antibody having an “enhanced ADCC activity” includes an antibody that is more effective at mediating ADCC in vitro or in vivo when compared to a control or parent antibody, wherein the antibody and the control/parent antibody differ in at least one structural aspect, and when the amounts of such antibody and control/parent antibody used in the assay are essentially the same. In some embodiments, the antibody and the control/parent antibody have the same amino acid sequence, but the antibody is afucosylated while the parent antibody is fucosylated. ADCC activity can be determined using any art recognized methods. In some embodiments, ADCC activity is determined using the in vitro ADCC assay as herein disclosed, but other assays or methods for determining ADCC activity, e.g., in an animal model etc., are contemplated. In some embodiments, an antibody with enhanced ADCC activity has enhanced affinity for Fc gamma RIIIA In some embodiments, an antibody with enhanced ADCC activity has enhanced affinity for Fc gamma RIIIA (V158). In some embodiments, an antibody with enhanced ADCC activity has enhanced affinity for Fc gamma RIIIA (F158).

An antibody with “altered” FcR binding affinity or ADCC activity is one which has either enhanced or diminished FcR binding activity, and/or ADCC activity compared to a control/parent antibody, wherein the antibody and the parent antibody differ in at least one structural aspect.

An antibody that “displays increased binding” to an FcR binds at least one FcR with better affinity than the parent antibody.

An antibody that “displays decreased binding” to an FcR, binds at least one FcR with lower affinity than a parent antibody. Such antibodies that display decreased binding to an FcR may possess little or no appreciable binding to an FcR, e.g., 0-20 percent binding to the FcR compared to a native sequence IgG Fc region.

“Enhanced affinity for Fc gamma RIIIA” refers to an antibody that has greater affinity for Fc gamma RIIIA (also referred to, in some instances, as CD 16a) than a control/parent antibody, wherein the antibody and the parent antibody differ in at least one structural aspect. In some embodiments, the antibody and the control/parent antibody have the same amino acid sequence, but the antibody is afucosylated while the control/parent antibody is fucosylated. Any suitable method for determining affinity for Fc gamma RIIIA may be used. In some embodiments, affinity for Fc gamma RIIIA is determined by a method described herein. In some embodiments, an antibody with enhanced affinity for Fc gamma RIIIA has enhanced ADCC activity. In some embodiments, an antibody with enhanced affinity for Fc gamma RIIIA has enhanced affinity for Fc gamma RIIIA(V158). In some embodiments, an antibody with enhanced affinity for Fc gamma RIIIA has enhanced affinity for Fc gamma RIIIA(F158).

“Complement dependent cytotoxicity” or “CDC” refers to the lysis of a target cell in the presence of complement. Activation of the classical complement pathway is initiated by the binding of the first component of the complement system (Clq) to antibodies (of the appropriate subclass), which are bound to their cognate antigen. To assess complement activation, a CDC assay, e.g., as described in Gazzano-Santoro et al., J. Immunol. Methods 202: 163, 1996, may be performed. Antibodies with altered Fc region amino acid sequences and increased or decreased Clq binding capability are described, e.g., in U.S. Pat. No. 6,194,551 B 1, U.S. Pat. Nos. 7,923,538, 7,994,290 and WO 1999/51642.

A “host cell” refers to a cell that may be or has been a recipient of a vector or isolated polynucleotide. Host cells may be prokaryotic cells or eukaryotic cells. Exemplary eukaryotic cells include mammalian cells, such as primate or non-primate animal cells; fungal cells, such as yeast; plant cells; and insect cells. Non-limiting exemplary mammalian cells include, but are not limited to, NS0 cells, PER.C6® cells (Crucell), and 293 and CHO cells, and their derivatives, such as 293-6E and DG44 cells, respectively.

The term “isolated” as used herein refers to a molecule that has been separated from at least some of the components with which it is typically found in nature or has been separated from at least some of the components with which it is typically produced. For example, a polypeptide is referred to as “isolated” when it is separated from at least some of the components of the cell in which it was produced. Where a polypeptide is secreted by a cell after expression, physically separating the supernatant containing the polypeptide from the cell that produced it is considered to be “isolating” the polypeptide. Similarly, a polynucleotide is referred to as “isolated” when it is not part of the larger polynucleotide (such as, for example, genomic DNA or mitochondrial DNA, in the case of a DNA polynucleotide) in which it is typically found in nature, or is separated from at least some of the components of the cell in which it was produced, e.g., in the case of an RNA polynucleotide. Thus, a DNA polynucleotide that is contained in a vector inside a host cell may be referred to as “isolated” so long as that polynucleotide is not found in that vector in nature.

The terms “subject” and “patient” are used interchangeably herein to refer to a mammal such as human. In some embodiments, methods of treating other non-human mammals, including, but not limited to, rodents, simians, felines, canines, equines, bovines, porcines, ovines, caprines, mammalian laboratory animals, mammalian farm animals, mammalian sport animals, and mammalian pets, are also provided. In some instances, a “subject” or “patient” refers to a (human) subject or patient in need of treatment for a disease or disorder.

The term “sample” or “patient sample” as used herein, refers to material that is obtained or derived from a subject of interest that contains a cellular and/or other molecular entity that is to be characterized and/or identified, for example based on physical, biochemical, chemical and/or physiological characteristics. For example, the phrase “disease sample” and variations thereof refers to any sample obtained from a subject of interest that would be expected or is known to contain the cellular and/or molecular entity that is to be characterized.

By “tissue or cell sample” is meant a collection of similar cells obtained from a tissue of a subject or patient. The source of the tissue or cell sample may be solid tissue as from a fresh, frozen and/or preserved organ or tissue sample or biopsy or aspirate; blood or any blood constituents; bodily fluids such as sputum, cerebral spinal fluid, amniotic fluid, peritoneal fluid, or interstitial fluid; cells from any time in gestation or development of the subject. The tissue sample may also be primary or cultured cells or cell lines. Optionally, the tissue or cell sample is obtained from a disease tissue/organ. The tissue sample may contain compounds which are not naturally intermixed with the tissue in nature such as preservatives, anticoagulants, buffers, fixatives, nutrients, antibiotics, or the like.

A “reference sample,” “reference cell,” or “reference tissue,” as used herein, refers to a sample, cell or tissue obtained from a source known, or believed, not to be afflicted with the disease or condition for which a method or composition of the invention is being used to identify. In one embodiment, a reference sample, reference cell or reference tissue is obtained from a healthy part of the body of the same subject or patient in whom a disease or condition is being identified using a composition or method of the invention. In one embodiment, a reference sample, reference cell or reference tissue is obtained from a healthy part of the body of at least one individual who is not the subject or patient in whom a disease or condition is being identified using a composition or method of the invention. In some embodiments, a reference sample, reference cell or reference tissue was previously obtained from a patient prior to developing a disease or condition or at an earlier stage of the disease or condition.

A “disorder” or “disease” is any condition that would benefit from treatment with one or more Gal-9 antagonists of the invention. This includes chronic and acute disorders or diseases including those pathological conditions that predispose the mammal to the disorder in question. Non-limiting examples of disorders to be treated herein include cancers.

The term “cancer” is used herein to refer to a group of cells that exhibit abnormally high levels of proliferation and growth. A cancer may be benign (also referred to as a benign tumor), pre-malignant, or malignant. Cancer cells may be solid cancer cells (i.e., forming solid tumors) or leukemic cancer cells. The term “cancer growth” is used herein to refer to proliferation or growth by a cell or cells that comprise a cancer that leads to a corresponding increase in the size or extent of the cancer.

Examples of cancer include but are not limited to, carcinoma, lymphoma, blastoma, sarcoma, and leukemia. More particular nonlimiting examples of such cancers include squamous cell cancer, small-cell lung cancer, pituitary cancer, esophageal cancer, astrocytoma, soft tissue sarcoma, non-small cell lung cancer, adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney cancer, renal cancer, liver cancer, prostate cancer, vulval cancer, thyroid cancer, hepatic carcinoma, brain cancer, endometrial cancer, testis cancer, cholangiocarcinoma, gallbladder carcinoma, gastric cancer, melanoma, and various types of head and neck cancer.

In certain embodiments, cancer as used herein includes a hematological cancer (such as lymphoma or leukemia, including CLL, Burkitt's lymphoma), or a solid tumor (such as breast cancer, lung cancer, and prostate cancer).

A “chemotherapeutic agent” is a chemical compound that can be useful in the treatment of cancer. Examples of chemotherapeutic agents include, but are not limited to, alkylating agents such as thiotepa and CYTOXAN® cyclosphosphamide; alkyl sulfonates such as busulfan, improsulfan and piposulfan; aziridines such as benzodopa, carboquone, meturedopa, and uredepa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammall and calicheamicin omegall (see, e.g., Agnew, Chem Intl. Ed. Engl, 33: 183-186 (1994)); dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, ADRIAMYCIN® doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfomithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, OR); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL® paclitaxel (Bristol-Myers Squibb Oncology, Princeton, N.J.), ABRAXANE® Cremophor-free, albumin-engineered nanoparticle formulation of paclitaxel (American Pharmaceutical Partners, Schaumberg, Illinois), and TAXOTERE® doxetaxel (Rhone-Poulenc Rorer, Antony, France); chloranbucil; GEMZAR® gemcitabine; 6-thioguanine; mercaptopurine; methotrexate; platinum analogs such as cisplatin, oxaliplatin and carboplatin; vinblastine; platinum; etoposide (VP-16); ifosfamide; mitoxantrone; vincristine; NAVELBINE® vinorelbine; novantrone; teniposide; edatrexate; daunomycin; aminopterin; xeloda; ibandronate; irinotecan (Camptosar, CPT-11) (including the treatment regimen of irinotecan with 5-FU and leucovorin); topoisomerase inhibitor RFS 2000; difluoromethylornithine (DMFO); retinoids such as retinoic acid; capecitabine; combretastatin; leucovorin (LV); oxaliplatin, including the oxaliplatin treatment regimen (FOLFOX); inhibitors of PKC-alpha, Raf, H-Ras, EGFR (e.g., erlotinib (TARCEVA®)) and VEGF-A that reduce cell proliferation and pharmaceutically acceptable salts, acids or derivatives of any of the above.

Further non-limiting exemplary chemotherapeutic agents include anti-hormonal agents that act to regulate or inhibit hormone action on cancers such as anti-estrogens and selective estrogen receptor modulators (SERMs), including, for example, tamoxifen (including NOLVADEX® tamoxifen), raloxifene, droloxifene, 4-hydroxy tamoxifen, trioxifene, keoxifene, LY117018, onapristone, and FARESTON® toremifene; aromatase inhibitors that inhibit the enzyme aromatase, which regulates estrogen production in the adrenal glands, such as, for example, 4(5)-imidazoles, aminoglutethimide, MEGASE® megestrol acetate, AROMASINT® exemestane, formestanie, fadrozole, RIVISOR® vorozole, FEMARA® letrozole, and ARIMIDEX® anastrozole; and anti-androgens such as flutamide, nilutamide, bicalutamide, leuprolide, and goserelin; as well as troxacitabine (a 1,3-dioxolane nucleoside cytosine analog); antisense oligonucleotides, particularly those which inhibit expression of genes in signaling pathways implicated in abherant cell proliferation, such as, for example, PKC-alpha, Ralf and H-Ras; ribozymes such as a VEGF expression inhibitor (e.g., ANGIOZYME® ribozyme) and a HER2 expression inhibitor; vaccines such as gene therapy vaccines, for example, ALLOVECTIN® vaccine, LEUVECTIN® vaccine, and VAXID® vaccine; PROLEUKIN® rIL-2; LURTOTECAN® topoisomerase 1 inhibitor; ABARELIX® rmRH; and pharmaceutically acceptable salts, acids or derivatives of any of the above.

An “anti-angiogenesis agent” or “angiogenesis inhibitor” refers to a small molecular weight substance, a polynucleotide (including, e.g., an inhibitory RNA (RNAi or siRNA)), a polypeptide, an isolated protein, a recombinant protein, an antibody, or conjugates or fusion proteins thereof, that inhibits angiogenesis, vasculogenesis, or undesirable vascular permeability, either directly or indirectly. It should be understood that the anti-angiogenesis agent includes those agents that bind and block the angiogenic activity of the angiogenic factor or its receptor. For example, an anti-angiogenesis agent is an antibody or other antagonist to an angiogenic agent, e.g., antibodies to VEGF-A (e.g., bevacizumab (AVASTIN®)) or to the VEGF-A receptor (e.g., KDR receptor or Flt-1 receptor), anti-PDGFR inhibitors such as GLEEVEC® (Imatinib Mesylate), small molecules that block VEGF receptor signaling (e.g., PTK787/ZK2284, SU6668, SUTENT®/SU1 1248 (sunitinib malate), AMG706, or those described in, e.g., international patent application WO 2004/113304). Anti-angiogenesis agents also include native angiogenesis inhibitors, e.g., angiostatin, endostatin, etc. See, e.g., Klagsbrun and D'Amore (1991) Annu. Rev. Physiol. 53:217-39; Streit and Detmar (2003) Oncogene 22:3172-3179 (e.g., Table 3 listing anti-angiogenic therapy in malignant melanoma); Ferrara & Alitalo (1999) Nature Medicine 5(12): 1359-1364; Tonini et al. (2003) Oncogene 22:6549-6556 (e.g., Table 2 listing known anti-angiogenic factors); and, Sato (2003) Int. J. Clin. Oncol. 8:200-206 (e.g., Table 1 listing anti-angiogenic agents used in clinical trials).

A “growth inhibitory agent” as used herein refers to a compound or composition that inhibits growth of a cell (such as a cell expressing VEGF) either in vitro or in vivo. Thus, the growth inhibitory agent may be one that significantly reduces the percentage of cells (such as a cell expressing VEGF) in S phase. Examples of growth inhibitory agents include, but are not limited to, agents that block cell cycle progression (at a place other than S phase), such as agents that induce G1 arrest and M-phase arrest. Classical M-phase blockers include the vincas (vincristine and vinblastine), taxanes, and topoisomerase II inhibitors such as doxorubicin, epirubicin, daunorubicin, etoposide, and bleomycin. Those agents that arrest G1 also spill over into S-phase arrest, for example, DNA alkylating agents such as tamoxifen, prednisone, dacarbazine, mechlorethamine, cisplatin, methotrexate, 5-fluorouracil, and ara-C. Further information can be found in Mendelsohn and Israel, eds., The Molecular Basis of Cancer, Chapter 1, entitled “Cell cycle regulation, oncogenes, and antineoplastic drugs” by Murakami et al. (W.B. Saunders, Philadelphia, 1995), e.g., p. 13. The taxanes (paclitaxel and docetaxel) are anticancer drugs both derived from the yew tree. Docetaxel (TAXOTERE®, Rhone-Poulenc Rorer), derived from the European yew, is a semisynthetic analogue of paclitaxel (TAXOL®, Bristol-Myers Squibb). Paclitaxel and docetaxel promote the assembly of microtubules from tubulin dimers and stabilize microtubules by preventing depolymerization, which results in the inhibition of mitosis in cells.

The term “anti-neoplastic composition” refers to a composition useful in treating cancer comprising at least one active therapeutic agent. Examples of therapeutic agents include, but are not limited to, e.g., chemotherapeutic agents, growth inhibitory agents, cytotoxic agents, agents used in radiation therapy, anti-angiogenesis agents, cancer immunotherapeutic agents (also referred to as immuno-oncology agents), apoptotic agents, anti-tubulin agents, and other-agents to treat cancer, such as anti-HER-2 antibodies, anti-CD20 antibodies, an epidermal growth factor receptor (EGFR) antagonist (e.g., a tyrosine kinase inhibitor), HER1/EGFR inhibitor (e.g., erlotinib (TARCEVA®), platelet derived growth factor inhibitors (e.g., GLEEVEC® (Imatinib Mesylate)), a COX-2 inhibitor (e.g., celecoxib), interferons, CTLA4 inhibitors (e.g., anti-CTLA antibody ipilimumab (YERVOY®)), PD-1 inhibitors (e.g., anti-PD1 antibodies, BMS-936558), PDL1 inhibitors (e.g., anti-PDL1 antibodies, MPDL3280A), PDL2 inhibitors (e.g., anti-PDL2 antibodies), VISTA inhibitors (e.g., anti-VISTA antibodies), cytokines, antagonists (e.g., neutralizing antibodies) that bind to one or more of the following targets ErbB2, ErbB3, ErbB4, PDGFR-beta, BlyS, APRIL, BCMA, PD-1, PDL1, PDL2, CTLA4, VISTA, or VEGF receptor(s), TRAIL/Apo2, and other bioactive and organic chemical agents, etc. Combinations thereof are also included in the invention.

“Treatment” refers to therapeutic treatment, for example, wherein the object is to slow down (lessen) the targeted pathologic condition or disorder as well as, for example, wherein the object is to inhibit recurrence of the condition or disorder. “Treatment” covers any administration or application of a therapeutic for a disease (also referred to herein as a “disorder” or a “condition”) in a mammal, including a human, and includes inhibiting the disease or progression of the disease, inhibiting or slowing the disease or its progression, arresting its development, partially or fully relieving the disease, partially or fully relieving one or more symptoms of a disease, or restoring or repairing a lost, missing, or defective function; or stimulating an inefficient process. The term “treatment” also includes reducing the severity of any phenotypic characteristic and/or reducing the incidence, degree, or likelihood of that characteristic. Those in need of treatment include those already with the disorder as well as those at risk of recurrence of the disorder or those in whom a recurrence of the disorder is to be prevented or slowed down.

The term “effective amount” or “therapeutically effective amount” refers to an amount of a drug effective to treat a disease or disorder in a subject. In some embodiments, an effective amount refers to an amount effective, at dosages and for periods of time necessary, to achieve the desired therapeutic or prophylactic result. A therapeutically effective amount of the antibodies of the invention may vary according to factors such as the disease state, age, sex, and weight of the individual, and the ability of the antagonist to elicit a desired response in the individual. A therapeutically effective amount encompasses an amount in which any toxic or detrimental effects of the subject antibodies are outweighed by the therapeutically beneficial effects.

A “prophylactically effective amount” refers to an amount effective, at dosages and for periods of time necessary, to achieve the desired prophylactic result. Typically, but not necessarily, since a prophylactic dose is used in subjects prior to or at an earlier stage of disease, the prophylactically effective amount would be less than the therapeutically effective amount.

A “pharmaceutically acceptable carrier” refers to a non-toxic solid, semisolid, or liquid filler, diluent, encapsulating material, formulation auxiliary, or carrier conventional in the art for use with a therapeutic agent that together comprise a “pharmaceutical composition” for administration to a subject. A pharmaceutically acceptable carrier is non-toxic to recipients at the dosages and concentrations employed and is compatible with other ingredients of the formulation. The pharmaceutically acceptable carrier is appropriate for the formulation employed. For example, if the therapeutic agent is to be administered orally, the carrier may be a gel capsule. If the therapeutic agent is to be administered subcutaneously, the carrier ideally is not irritable to the skin and does not cause injection site reaction.

An “article of manufacture” is any manufacture (e.g., a package or container) or kit comprising at least one reagent, e.g., a medicament for treatment of a disease or disorder, or a probe for specifically detecting a biomarker described herein. In some embodiments, the manufacture or kit is promoted, distributed, or sold as a unit for performing the methods described herein.

### 3. Methods of Use, Such as Methods of Treating Cancer

The invention described herein provides anti-CXCR5 antibodies for use in methods of treating humans and other non-human mammals.

In some embodiments, methods for treating or preventing a cancer are provided, comprising administering an effective amount of any of the subject anti-CXCR5 antibodies or antigen-binding fragments thereof to a subject in need of such treatment.

In some embodiments, methods of treating cancer are provided, wherein the methods comprise administering any of the subject anti-CXCR5 antibodies or antigen-binding fragments thereof to a subject with cancer.

The cancers treatable by the method/use of the invention include any of those described herein/above.

Non-limiting exemplary cancers that may be treated with any of the subject anti-CXCR5 antibodies or antigen-binding fragments thereof are provided herein, including carcinoma, lymphoma, blastoma, sarcoma, and leukemia. More particular non-limiting examples of such cancers include melanoma, cervical cancer, squamous cell cancer, small-cell lung cancer, pituitary cancer, esophageal cancer, astrocytoma, soft tissue sarcoma, non-small cell lung cancer, adenocarcinoma of the lung, squamous carcinoma of the lung, cancer of the peritoneum, hepatocellular cancer, gastrointestinal cancer, pancreatic cancer, glioblastoma, ovarian cancer, liver cancer, bladder cancer, hepatoma, breast cancer, colon cancer, colorectal cancer, endometrial or uterine carcinoma, salivary gland carcinoma, kidney cancer, renal cancer, liver cancer, prostate cancer, vulval cancer, thyroid cancer, hepatic carcinoma, brain cancer, endometrial cancer, testis cancer, cholangiocarcinoma, gallbladder carcinoma, gastric cancer, melanoma, and various types of head and neck cancer.

In certain embodiment, the cancer is melanoma, breast cancer, colon cancer, cervical cancer, renal cancer, liver cancer (e.g., hepatocellular carcinoma), lung cancer (NSCLC), ovarian cancer, skin cancer (e.g., squamous cell carcinoma or basal cell carcinoma), lymphoma, or leukemia.

In some embodiments, the anti-CXCR5 antibodies of the invention can be used alone, or alternatively used in combination with any other suitable compound known to be able to treat the disease or indication, for example, an anticancer agent.

That is, when the use is the treatment of a cancer, the antibody can be used in combination with known therapies against cancer such as for example surgery, radiotherapy, chemotherapy or combinations thereof. For example, the antibody can be used in combination with an adoptive immunotherapy, consisting one or more injections of effector lymphocytes against tumoral antigens, in particular EBV antigens. According to some aspects, other anticancer agents used in combination with the antibody directed against CXCR5 according to the invention for cancer therapy comprise anti-angiogenics. According to certain aspects, the antibody can be co-administered with a cytokine, for example a cytokine that stimulates an anti-tumoral immune response.

In such combination therapy, the antibody of the invention can be used before, after, or concurrently with the second therapeutic agent. See further section below concerning combination therapy.

A related aspect of the invention provides a method of reducing the activity of CXCR5, comprising administering to a subject in need thereof a therapeutically effective amount of the antibody, or antigen-binding fragment thereof of the invention as described herein, or the pharmaceutical composition thereof.

A related aspect of the invention provides a method of treating an inflammatory disease, comprising administering to a subject in need thereof a therapeutically effective amount of the antibody, or antigen-binding fragment thereof of the invention, or the pharmaceutical composition thereof.

A related aspect of the invention provides a method of treating a subject in need of immunosuppression comprising administering to a subject in need thereof a therapeutically effective amount of the antibody, or antigen-binding fragment thereof of the invention, or the pharmaceutical composition thereof.

A related aspect of the invention provides a method of treating an autoimmune disease, disorder or condition, comprising administering to a subject in need thereof a therapeutically effective amount of the antibody, or antigen-binding fragment thereof of the invention, or the pharmaceutical composition thereof.

A related aspect of the invention provides a method of decreasing the number of cells expressing CXCR5 in a subject in need thereof, said method comprising administering to the subject a therapeutically effective amount of the antibody, or antigen-binding fragment thereof of the invention, or the pharmaceutical composition thereof.

In certain embodiments, the cells express CXCR5 on their surface. In certain embodiments, the cells are B cells and Tfh-like cells.

In certain embodiments, the subject is a human.

In certain embodiments, the method comprises administering the antibody or antigen-binding fragment thereof, or pharmaceutical composition thereof, intravenously, or subcutaneously.

In certain embodiments, the antibody or antigen-binding fragment thereof, or pharmaceutical composition, is administered about twice a week, once a week, once every two weeks, once every three weeks, once every four weeks, once every five weeks, once every six weeks, once every seven weeks, once every eight weeks, once every nine weeks, once every ten weeks, twice a month, once a month, once every two months, once every three months, once every four months, once every five months, once every six months, once every seven months, once every eight months, once every nine months, once every ten months, once every eleven months or once every twelve months.

A related aspect of the invention provides a method of decreasing the number of CXCR5+ cells in a sample, said method comprising contacting said cell with the antibody, or antigen-binding fragment thereof of the invention, or the pharmaceutical composition thereof.

A related aspect of the invention provides the antibody, or antigen-binding fragment thereof of the invention, or the pharmaceutical composition thereof, for use as a medicament.

A related aspect of the invention provides the antibody, or antigen-binding fragment thereof of the invention, or the pharmaceutical composition thereof, for use in reducing the activity of CXCR5 in a subject.

A related aspect of the invention provides the antibody, or antigen-binding fragment thereof of the invention, or the pharmaceutical composition thereof, for use in treating a subject in need of immunosuppression.

A related aspect of the invention provides the antibody, or antigen-binding fragment thereof of the invention, or the pharmaceutical composition thereof, for use in treating an autoimmune disease, disorder or condition in a subject.

A related aspect of the invention provides use of an antibody, or antigen-binding fragment thereof of the invention in the manufacture of a medicament for treating an immune disease, disorder or condition.

A related aspect of the invention provides use of a pharmaceutical composition of the invention in the manufacture of a medicament for treating an immune disease, disorder or condition.

A related aspect of the invention provides a method of treating a medical condition, comprising administering to a subject in need thereof a therapeutically effective amount of the antibody, or antigen-binding fragment thereof of the invention, or the pharmaceutical composition thereof.

In certain embodiments, the condition is selected from the group consisting of inflammatory responses such as inflammatory skin diseases including psoriasis and dermatitis (e. g. atopic dermatitis); dermatomyositis; systemic scleroderma and sclerosis; responses associated with inflammatory bowel disease (such as Crohn's disease and ulcerative colitis); respiratory distress syndrome (including adult respiratory distress syndrome; ARDS); dermatitis; meningitis; encephalitis; uveitis; colitis; gastritis; glomerulonephritis; allergic conditions such as eczema and asthma and other conditions involving infiltration of T cells and chronic inflammatory responses; atherosclerosis; leukocyte adhesion deficiency; rheumatoid arthritis; systemic lupus erythematosus (SLE); diabetes mellitus (e. g. Type I diabetes mellitus or insulin dependent diabetes mellitis); multiple sclerosis; Reynaud's syndrome; autoimmune thyroiditis; allergic encephalomyelitis; Sjogren's syndrome; juvenile onset diabetes; and immune responses associated with acute and delayed hypersensitivity mediated by cytokines and T-lymphocytes typically found in tuberculosis, sarcoidosis, polymyositis, granulomatosis and vasculitis; Wegener's disease; pernicious anemia (Addison's disease); diseases involving leukocyte diapedesis; central nervous system (CNS) inflammatory disorder; multiple organ injury syndrome; hemolytic anemia (including, but not limited to cryoglobinemia or Coombs positive anemia); myasthenia gravis; antigen-antibody complex mediated diseases; anti-glomerular basement membrane disease; antiphospholipid syndrome; allergic neuritis; Graves' disease; Lambert-Eaton myasthenic syndrome; pemphigoid bullous; pemphigus; autoimmune polyendocrinopathies; vitiligo; Reiter's disease; stiff-person syndrome; Bechet disease; giant cell arteritis; immune complex nephritis; IgA nephropathy; IgM polyneuropathies; immune thrombocytopenic purpura (ITP) or autoimmune thrombocytopenia and autoimmune hemolytic diseases; Hashimoto's thyroiditis; autoimmune hepatitis; autoimmune hemophilia; autoimmune lymphoproliferative syndrome (ALPS); autoimmune uveoretinitis; Guillain-Barre syndrome; Goodpasture's syndrome; mixed connective tissue disease; autoimmune-associated infertility; polyarteritis nodosa; alopecia areata; idiopathic myxedema; graft versus host disease; muscular dystrophy (Duchenne, Becker, Myotonic, Limb-girdle, Facioscapulohumeral, Congenital, Oculopharyngeal, Distal, Emery-Dreifuss) and controlling the proliferation of cancer cells expressing CXCR5 such as cancers of the pancreas, colon, bladder, T-cell leukemia, and B-cell leukemia. In certain embodiments, the disease is SLE or rheumatoid arthritis.

In certain embodiments, the disease is Sjogren's syndrome.

A related aspect of the invention provides a method of detecting CXCR5 in a sample, tissue, or cell using the antibody, or antigen-binding fragment thereof of the invention, or the pharmaceutical composition thereof, comprising contacting the sample, tissue or cell with the antibody and detecting the antibody.

A related aspect of the invention provides a method of detecting CXCR5 in a sample, tissue, or cell using the antibody, or antigen-binding fragment thereof of the invention, comprising contacting the sample, tissue or cell with the antibody and detecting the antibody.

A related aspect of the invention provides a method of inhibiting a humoral immune response in a subject in need thereof, the method comprising administering a therapeutically effective amount of the antibody, or antigen-binding fragment thereof of the invention, or the pharmaceutical composition thereof.

In certain embodiments, the antibody mediates depletion of at least one cell expressing CXCR5 selected from the group consisting of a Tfh cell in the spleen, a B cell in peripheral blood, and a Tfh-like cell in peripheral blood.

### 4. Routes of Administration and Carriers

In various embodiments, the subject anti-CXCR5 monoclonal antibodies may be administered subcutaneously or intravenously. For simplicity, “the subject anti-CXCR5 monoclonal antibodies” refer to mouse-human chimeric anti-CXCR5 antibody of the invention, as well as the humanized variants thereof.

In some embodiments, the subject anti-CXCR5 monoclonal antibodies may be administered in vivo by various routes, including, but not limited to, oral, intra-arterial, parenteral, intranasal, intramuscular, intracardiac, intraventricular, intratracheal, buccal, rectal, intraperitoneal, by inhalation, intradermal, topical, transdermal, and intrathecal, or otherwise, e.g., by implantation.

In some embodiments, the subject anti-CXCR5 monoclonal antibodies may be administered via i.v. or s.c.

The subject antibody compositions may be formulated into preparations in solid, semi-solid, liquid, or gaseous forms; including, but not limited to, tablets, capsules, powders, granules, ointments, solutions, suppositories, enemas, injections, inhalants, and aerosols.

In various embodiments, compositions comprising the subject anti-CXCR5 monoclonal antibodies are provided in formulations with a wide variety of pharmaceutically acceptable carriers (see, e.g., Gennaro, Remington: The Science and Practice of Pharmacy with Facts and Comparisons: Drugfacts Plus, 20th ed. (2003); Ansel et al., Pharmaceutical Dosage Forms and Drug Delivery Systems, 7th ed., Lippencott Williams and Wilkins (2004); Kibbe et al., Handbook of Pharmaceutical Excipients, 3rd ed., Pharmaceutical Press (2000)). Various pharmaceutically acceptable carriers, which include vehicles, adjuvants, and diluents, are available. Moreover, various pharmaceutically acceptable auxiliary substances, such as pH adjusting and buffering agents, tonicity adjusting agents, stabilizers, wetting agents and the like, are also available. Nonlimiting exemplary carriers include saline, buffered saline, dextrose, water, glycerol, ethanol, and combinations thereof.

In various embodiments, compositions comprising the subject anti-CXCR5 monoclonal antibodies may be formulated for injection, including subcutaneous administration, by dissolving, suspending, or emulsifying them in an aqueous or nonaqueous solvent, such as vegetable or other oils, synthetic aliphatic acid glycerides, esters of higher aliphatic acids, or propylene glycol; and if desired, with conventional additives such as solubilizers, isotonic agents, suspending agents, emulsifying agents, stabilizers and preservatives.

In various embodiments, the compositions may be formulated for inhalation, for example, using pressurized acceptable propellants such as dichlorodifluoromethane, propane, nitrogen, and the like.

The compositions may also be formulated, in various embodiments, into sustained release microcapsules, such as with biodegradable or non-biodegradable polymers. A non-limiting exemplary biodegradable formulation includes poly lactic acid-glycolic acid (PLGA) polymer. A non-limiting exemplary non-biodegradable formulation includes a polyglycerin fatty acid ester. Certain methods of making such formulations are described, for example, in EP 1125584 A1.

Pharmaceutical dosage packs comprising one or more containers, each containing one or more doses of the subject anti-CXCR5 monoclonal antibodies, are also provided. In some embodiments, a unit dosage is provided wherein the unit dosage contains a predetermined amount of a composition comprising the subject anti-CXCR5 monoclonal antibodies, with or without one or more additional agents. In some embodiments, such a unit dosage is supplied in single-use prefilled syringe for injection. In various embodiments, the composition contained in the unit dosage may comprise saline, sucrose, or the like; a buffer, such as phosphate, or the like; and/or be formulated within a stable and effective pH range. Alternatively, in some embodiments, the composition may be provided as a lyophilized powder that may be reconstituted upon addition of an appropriate liquid, for example, sterile water. In some embodiments, the composition comprises one or more substances that inhibit protein aggregation, including, but not limited to, sucrose and arginine. In some embodiments, a composition of the invention comprises heparin and/or a proteoglycan.

Pharmaceutical compositions are administered in an amount effective for treatment or prophylaxis of the specific indication. The therapeutically effective amount is typically dependent on the weight of the subject being treated, his or her physical or health condition, the extensiveness of the condition to be treated, or the age of the subject being treated.

In some embodiments, the subject anti-CXCR5 monoclonal antibodies may be administered in an amount in the range of about 50 μg/kg body weight to about 50 mg/kg body weight per dose. In some embodiments, the subject anti-CXCR5 monoclonal antibodies may be administered in an amount in the range of about 100 μg/kg body weight to about 50 mg/kg body weight per dose. In some embodiments, the subject anti-CXCR5 monoclonal antibodies may be administered in an amount in the range of about 100 μg/kg body weight to about 20 mg/kg body weight per dose. In some embodiments, the subject anti-CXCR5 monoclonal antibodies may be administered in an amount in the range of about 0.5 mg/kg body weight to about 20 mg/kg body weight per dose.

In some embodiments, the subject anti-CXCR5 monoclonal antibodies may be administered in an amount in the range of about 10 mg to about 1,000 mg per dose. In some embodiments, the subject anti-CXCR5 monoclonal antibodies may be administered in an amount in the range of about 20 mg to about 500 mg per dose. In some embodiments, the subject anti-CXCR5 monoclonal antibodies may be administered in an amount in the range of about 20 mg to about 300 mg per dose. In some embodiments, the subject anti-CXCR5 monoclonal antibodies may be administered in an amount in the range of about 20 mg to about 200 mg per dose.

The subject anti-CXCR5 monoclonal antibody compositions may be administered as needed to subjects. In some embodiments, an effective dose of the subject anti-CXCR5 monoclonal antibodies is administered to a subject one or more times. In various embodiments, an effective dose of the subject anti-CXCR5 monoclonal antibodies is administered to the subject once a month, less than once a month, such as, for example, every two months, every three months, or every six months. In other embodiments, an effective dose of the subject anti-CXCR5 monoclonal antibodies is administered more than once a month, such as, for example, every two weeks, every week, twice per week, three times per week, daily, or multiple times per day. An effective dose of the subject anti-CXCR5 monoclonal antibodies is administered to the subject at least once. In some embodiments, the effective dose of the subject anti-CXCR5 monoclonal antibodies may be administered multiple times, including for periods of at least a month, at least six months, or at least a year. In some embodiments, the subject anti-CXCR5 monoclonal antibodies is administered to a subject as-needed to alleviate one or more symptoms of a condition.

### 5. Combination Therapy

The subject anti-CXCR5 monoclonal antibodies of the invention, including functional fragments thereof, may be administered to a subject in need thereof in combination with other biologically active substances or other treatment procedures for the treatment of diseases. For example, the subject anti-CXCR5 monoclonal antibodies may be administered alone or with other modes of treatment. They may be provided before, substantially contemporaneous with, or after other modes of treatment, such as radiation therapy.

For treatment of cancer, the subject anti-CXCR5 monoclonal antibodies may be administered in conjunction with one or more of anti-cancer agents, such as the immune checkpoint inhibitor, chemotherapeutic agent, growth inhibitory agent, anti-angiogenesis agent or anti-neoplastic composition.

In certain embodiments, the subject anti-CXCR5 monoclonal antibodies specifically binds to CXCR5 (a “CXCR5-binding antagonist”), e.g., CXCR5 antagonist antibody or antigen-binding fragment thereof, is administered with a second antagonist such as an immune checkpoint inhibitor (e.g., an inhibitor of the PD-1 or PD-L1 pathway), to a subject having a disease in which the stimulation of the immune system would be beneficial, e.g., cancer or infectious diseases. The two antagonists may be administered simultaneously or consecutively, e.g., as described below for the combination of the subject anti-CXCR5 monoclonal antibodies with an immuno-oncology agent. One or more additional therapeutics, e.g., checkpoint modulators may be added to a treatment with the subject anti-CXCR5 monoclonal antibodies for treating cancer or autoimmune diseases.

In certain embodiments, the subject anti-CXCR5 monoclonal antibodies is administered with another treatment, either simultaneously, or consecutively, to a subject, e.g., a subject having cancer. For example, the subject anti-CXCR5 monoclonal antibodies may be administered with one of more of: radiotherapy, surgery, or chemotherapy, e.g., targeted chemotherapy or immunotherapy.

In certain embodiments, a method of treatment of a subject having cancer comprises administering to the subject an anti-CXCR5 monoclonal antibody of the invention, and one or more immuno-oncology agents, such as immune checkpoint inhibitor.

Immunotherapy, e.g., therapy with an immuno-oncology agent, is effective to enhance, stimulate, and/or upregulate immune responses in a subject. In one aspect, the administration of the subject anti-CXCR5 monoclonal antibodies with an immuno-oncology agent (such as a PD-1 inhibitor) has a synergic effect in the treatment of cancer, e.g., in inhibiting tumor growth.

In one aspect, a subject anti-CXCR5 monoclonal antibody is sequentially administered prior to administration of the immuno-oncology agent. In one aspect, a subject anti-CXCR5 monoclonal antibody is administered concurrently with the immunology-oncology agent (such as PD-1 inhibitor). In yet one aspect, a subject anti-CXCR5 monoclonal antibody is sequentially administered after administration of the immuno-oncology agent (such as PD-1 inhibitor). The administration of the two agents may start at times that are, e.g., 30 minutes, 60 minutes, 90 minutes, 120 minutes, 3 hours, 6 hours, 12 hours, 24 hours, 36 hours, 48 hours, 3 days, 5 days, 7 days, or one or more weeks apart, or administration of the second agent may start, e.g., 30 minutes, 60 minutes, 90 minutes, 120 minutes, 3 hours, 6 hours, 12 hours, 24 hours, 36 hours, 48 hours, 3 days, 5 days, 7 days, or one or more weeks after the first agent has been administered.

In certain aspects, the subject anti-CXCR5 monoclonal antibodies and an immuno-oncology agent (e.g., PD-1 inhibitor) are administered simultaneously, e.g., are infused simultaneously, e.g., over a period of 30 or 60 minutes, to a patient. The subject anti-CXCR5 monoclonal antibodies may be co-formulated with an immuno-oncology agent (such as PD-1 inhibitor).

Immuno-oncology agents include, for example, a small molecule drug, antibody or fragment thereof, or other biologic or small molecule. Examples of biologic immuno-oncology agents include, but are not limited to, antibodies, antibody fragments, vaccines and cytokines. In one aspect, the antibody is a monoclonal antibody. In certain aspects, the monoclonal antibody is humanized or human antibody.

In one aspect, the immuno-oncology agent is (i) an agonist of a stimulatory (including a co-stimulatory) molecule (e.g., receptor or ligand) or (ii) an antagonist of an inhibitory (including a co-inhibitory) molecule (e.g., receptor or ligand) on immune cells, e.g., T cells, both of which result in amplifying antigen-specific T cell responses. In certain aspects, an immuno-oncology agent is (i) an agonist of a stimulatory (including a co-stimulatory) molecule (e.g., receptor or ligand) or (ii) an antagonist of an inhibitory (including a co-inhibitory) molecule (e.g., receptor or ligand) on cells involved in innate immunity, e.g., NK cells, and wherein the immuno-oncology agent enhances innate immunity. Such immuno-oncology agents are often referred to as immune checkpoint regulators, e.g., immune checkpoint inhibitor or immune checkpoint stimulator.

In certain embodiments, the immuno-oncology agent may be an agent that targets (or binds specifically to) a member of the B7 family of membrane-bound ligands, which includes B7-1, B7-2, B7-H1 (PD-L1), B7-DC (PD-L2), B7-H2 (ICOS-L), B7-H3, B7-H4, B7-H5, and B7-H6, or a co-stimulatory or co-inhibitory receptor binding specifically to a B7 family member. An immuno-oncology agent may be an agent that targets a member of the TNF family of membrane bound ligands or a co-stimulatory or co-inhibitory receptor binding specifically thereto, e.g., a TNF receptor family member. Exemplary TNF and TNFR family members that may be targeted by immuno-oncology agents include CD40 and CD40L, OX-40, OX-40L, GITR, GITRL, CD70, CD27L, CD30, CD30L, 4-1BBL, CD137 (4-1BB), TRAIL/Apo2-L, TRAILR1/DR4, TRAILR2/DR5, TRAILR3, TRAILR4, OPG, RANK, RANKL, TWEAKR/Fn14, TWEAK, BAFFR, EDAR, XEDAR, TACI, APRIL, BCMA, LTfiR, LIGHT, DcR3, HVEM, VEGI/TL1A, TRAMP/DR3, EDAR, EDA1, XEDAR, EDA2, TNFR1, Lymphotoxin α/TNPβ, CXCR5, TNFa, LTfiR, Lymphotoxin a 1(32, FAS, FASL, RELT, DR6, TROY and NGFR. An immuno-oncology agent that may be used in combination with the subject anti-CXCR5 monoclonal antibodies for treating cancer may be an agent, e.g., an antibody, targeting a B7 family member, a B7 receptor family member, a TNF family member or a TNFR family member, such as those described above.

In one aspect, a subject anti-CXCR5 monoclonal antibody is administered with one or more of (i) an antagonist of a protein that inhibits T cell activation (e.g., immune checkpoint inhibitor) such as CTLA-4, PD-1, PD-L1, PD-L2, LAG-3, TIM3, CEACAM-1, BTLA, CD69, Galectin-1, TIGIT, CD113, GPR56, VISTA, B7-H3, B7-H4, 2B4, CD48, GARP, PDIH, LAIR1, TIM-1, TIM-4, and PSGL-1 and (ii) an agonist of a protein that stimulates T cell activation such as B7-1, B7-2, CD28, 4-1BB (CD137), 4-1BBL, ICOS, ICOS-L, OX40, OX40L, GITR, GITRL, CD70, CD27, CD40, CD40L, DR3 and CD28H.

In one aspect, an immuno-oncology agent is an agent that inhibits (i.e., an antagonist of) a cytokine that inhibits T cell activation (e.g., IL-6, IL-10, TGF-β, VEGF, and other immunosuppressive cytokines) or is an agonist of a cytokine, such as IL-2, IL-7, IL-12, IL-15, IL-21 and IFNα (e.g., the cytokine itself) that stimulates T cell activation, and stimulates an immune response.

Other agents that can be combined with the subject anti-CXCR5 monoclonal antibodies for stimulating the immune system, e.g., for the treatment of cancer and infectious diseases, include antagonists of inhibitory receptors on NK cells or agonists of activating receptors on NK cells. For example, the subject anti-CXCR5 monoclonal antibodies can be combined with an antagonist of KIR.

Yet other agents for combination therapies include agents that inhibit or deplete macrophages or monocytes, including but not limited to CSF-IR antagonists such as CSF-IR antagonist antibodies including RG7155 (WO11/70024, WO11/107553, WO11/131407, WO13/87699, WO13/119716, WO13/132044) or FPA008 (WO11/140249; WO13169264; WO14/036357).

Immuno-oncology agents also include agents that inhibit TGF-β signaling.

Additional agents that may be combined with the subject anti-CXCR5 monoclonal antibodies include agents that enhance tumor antigen presentation, e.g., dendritic cell vaccines, GM-CSF secreting cellular vaccines, CpG oligonucleotides, and imiquimod, or therapies that enhance the immunogenicity of tumor cells (e.g., anthracyclines).

Yet other therapies that may be combined with the subject anti-CXCR5 monoclonal antibodies include therapies that deplete or block T_{reg} cells, e.g., an agent that specifically binds to CD25.

Another therapy that may be combined with the subject anti-CXCR5 monoclonal antibodies is a therapy that inhibits a metabolic enzyme such as indoleamine dioxigenase (IDO), dioxigenase, arginase, or nitric oxide synthetase.

Another class of agents that may be used includes agents that inhibit the formation of adenosine or inhibit the adenosine A2A receptor.

Other therapies that may be combined with the subject anti-CXCR5 monoclonal antibodies for treating cancer include therapies that reverse/prevent T cell anergy or exhaustion and therapies that trigger an innate immune activation and/or inflammation at a tumor site.

The subject anti-CXCR5 monoclonal antibodies may be combined with more than one immuno-oncology agent (such as immune checkpoint inhibitor), and may be, e.g., combined with a combinatorial approach that targets multiple elements of the immune pathway, such as one or more of the following: a therapy that enhances tumor antigen presentation (e.g., dendritic cell vaccine, GM-CSF secreting cellular vaccines, CpG oligonucleotides, imiquimod); a therapy that inhibits negative immune regulation e.g., by inhibiting CTLA-4 and/or PD1/PD-L1/PD-L2 pathway and/or depleting or blocking Treg or other immune suppressing cells; a therapy that stimulates positive immune regulation, e.g., with agonists that stimulate the CD-137, OX-40 and/or GITR pathway and/or stimulate T cell effector function; a therapy that increases systemically the frequency of anti-tumor T cells; a therapy that depletes or inhibits Tregs, such as Tregs in the tumor, e.g., using an antagonist of CD25 (e.g., daclizumab) or by ex vivo anti-CD25 bead depletion; a therapy that impacts the function of suppressor myeloid cells in the tumor; a therapy that enhances immunogenicity of tumor cells (e.g., anthracyclines); adoptive T cell or NK cell transfer including genetically modified cells, e.g., cells modified by chimeric antigen receptors (CAR-T therapy); a therapy that inhibits a metabolic enzyme such as indoleamine dioxigenase (IDO), dioxigenase, arginase or nitric oxide synthetase; a therapy that reverses/prevents T cell anergy or exhaustion; a therapy that triggers an innate immune activation and/or inflammation at a tumor site; administration of immune stimulatory cytokines or blocking of immuno repressive cytokines.

For example, the subject anti-CXCR5 monoclonal antibodies can be used with one or more agonistic agents that ligate positive costimulatory receptors; one or more antagonists (blocking agents) that attenuate signaling through inhibitory receptors, such as antagonists that overcome distinct immune suppressive pathways within the tumor microenvironment (e.g., block PD-L1/PD-1/PD-L2 interactions); one or more agents that increase systemically the frequency of anti-tumor immune cells, such as T cells, deplete or inhibit Tregs (e.g., by inhibiting CD25); one or more agents that inhibit metabolic enzymes such as IDO; one or more agents that reverse/prevent T cell anergy or exhaustion; and one or more agents that trigger innate immune activation and/or inflammation at tumor sites.

In one embodiment, a subject having a disease that may benefit from stimulation of the immune system, e.g., cancer or an infectious disease, is treated by administration to the subject of the subject anti-CXCR5 monoclonal antibodies and an immuno-oncology agent, wherein the immuno-oncology agent is a CTLA-4 antagonist, such as an antagonistic CTLA-4 antibody. Suitable CTLA-4 antibodies include, for example, YERVOY (ipilimumab) or tremelimumab.

In one embodiment, a subject having a disease that may benefit from stimulation of the immune system, e.g., cancer or an infectious disease, is treated by administration to the subject of the subject anti-CXCR5 monoclonal antibodies and an immuno-oncology agent, wherein the immuno-oncology agent is a PD-1 antagonist, such as an antagonistic PD-1 antibody. Suitable PD-1 antibodies include, for example, OPDIVO (nivolumab), KEYTRUDA (pembrolizumab), or MEDI-0680 (AMP-514; WO2012/145493). The immuno-oncology agent may also include pidilizumab (CT-011). Another approach to target the PD-1 receptor is the recombinant protein composed of the extracellular domain of PD-L2 (B7-DC) fused to the Fc portion of IgG1, called AMP-224.

In one embodiment, a subject having a disease that may benefit from stimulation of the immune system, e.g., cancer or an infectious disease, is treated by administration to the subject of an anti-CXCR5 monoclonal antibody of the invention and an immuno-oncology agent, wherein the immuno-oncology agent is a PD-L1 antagonist, such as an antagonistic PD-L1 antibody. Suitable PD-L1 antibodies include, for example, MPDL3280A (RG7446; WO2010/077634), durvalumab (MEDI4736), BMS-936559 (WO2007/005874), MSB0010718C (WO2013/79174) or rHigM12B7.

In one embodiment, a subject having a disease that may benefit from stimulation of the immune system, e.g., cancer or an infectious disease, is treated by administration to the subject an anti-CXCR5 monoclonal antibody of the invention and an immuno-oncology agent, wherein the immuno-oncology agent is a LAG-3 antagonist, such as an antagonistic LAG-3 antibody. Suitable LAG3 antibodies include, for example, BMS-986016 (WO10/19570, WO14/08218), or IMP-731 or IMP-321 (WO08/132601, WO09/44273).

In one embodiment, a subject having a disease that may benefit from stimulation of the immune system, e.g., cancer or an infectious disease, is treated by administration to the subject an anti-CXCR5 monoclonal antibody of the invention and an immuno-oncology agent, wherein the immuno-oncology agent is a CD137 (4-1BB) agonist, such as an agonistic CD137 antibody. Suitable CD137 antibodies include, for example, urelumab or PF-05082566 (WO12/32433).

In one embodiment, a subject having a disease that may benefit from stimulation of the immune system, e.g., cancer or an infectious disease, is treated by administration to the subject an anti-CXCR5 monoclonal antibody of the invention and an immuno-oncology agent, wherein the immuno-oncology agent is a GITR agonist, such as an agonistic GITR antibody. Suitable GITR antibodies include, for example, TRX-518 (WO06/105021, WO09/009116), MK-4166 (WO 11/028683) or a GITR antibody disclosed in WO2015/031667.

In one embodiment, a subject having a disease that may benefit from stimulation of the immune system, e.g., cancer or an infectious disease, is treated by administration to the subject an anti-CXCR5 monoclonal antibody of the invention and an immuno-oncology agent, wherein the immuno-oncology agent is an OX40 agonist, such as an agonistic OX40 antibody. Suitable OX40 antibodies include, for example, MEDI-6383, MEDI-6469 or MOXR0916 (RG7888; WO06/029879).

In one embodiment, a subject having a disease that may benefit from stimulation of the immune system, e.g., cancer or an infectious disease, is treated by administration to the subject an anti-CXCR5 monoclonal antibody of the invention and an immuno-oncology agent, wherein the immuno-oncology agent is a CD40 agonist, such as an agonistic CD40 antibody. In certain embodiments, the immuno-oncology agent is a CD40 antagonist, such as an antagonistic CD40 antibody. Suitable CD40 antibodies include, for example, lucatumumab (HCD122), dacetuzumab (SGN-40), CP-870,893 or Chi Lob 7/4.

In one embodiment, a subject having a disease that may benefit from stimulation of the immune system, e.g., cancer or an infectious disease, is treated by administration to the subject an anti-CXCR5 monoclonal antibody of the invention and an immuno-oncology agent, wherein the immuno-oncology agent is a CD27 agonist, such as an agonistic CD27 antibody. Suitable CD27 antibodies include, for example, varlilumab (CDX-1127).

In one embodiment, a subject having a disease that may benefit from stimulation of the immune system, e.g., cancer or an infectious disease, is treated by administration to the subject an anti-CXCR5 monoclonal antibody of the invention and an immuno-oncology agent, wherein the immuno-oncology agent is MGA271 (to B7H3) (WO1 1/109400).

In one embodiment, a subject having a disease that may benefit from stimulation of the immune system, e.g., cancer or an infectious disease, is treated by administration to the subject an anti-CXCR5 monoclonal antibody of the invention and an immuno-oncology agent, wherein the immuno-oncology agent is a KIR antagonist, such as lirilumab.

In one embodiment, a subject having a disease that may benefit from stimulation of the immune system, e.g., cancer or an infectious disease, is treated by administration to the subject an anti-CXCR5 monoclonal antibody of the invention and an immuno-oncology agent, wherein the immuno-oncology agent is an IDO antagonist. Suitable IDO antagonists include, for example, INCB-024360 (WO2006/122150, WO07/75598, WO08/36653, WO08/36642), indoximod, NLG-919 (WO09/73620, WO09/1156652, WO1 1/56652, WO 12/142237) or F001287.

In one embodiment, a subject having a disease that may benefit from stimulation of the immune system, e.g., cancer or an infectious disease, is treated by administration to the subject an anti-CXCR5 monoclonal antibody of the invention and an immuno-oncology agent, wherein the immuno-oncology agent is a Toll-like receptor agonist, e.g., a TLR2/4 agonist (e.g., Bacillus Calmette-Guerin); a TLR7 agonist (e.g., Hiltonol or Imiquimod); a TLR7/8 agonist (e.g., Resiquimod); or a TLR9 agonist (e.g., CpG7909).

In one embodiment, a subject having a disease that may benefit from stimulation of the immune system, e.g., cancer or an infectious disease, is treated by administration to the subject an anti-CXCR5 monoclonal antibody of the invention and an immuno-oncology agent, wherein, the immuno-oncology agent is a TGF-β inhibitor, e.g., GC1008, LY2157299, TEW7197 or IMC-TR1.

### 6. Exemplary Anti-CXCR5 Monoclonal Antibody

The invention described herein provides monoclonal antibodies specific for CXCR5, or antigen-binding fragments thereof.

Thus one aspect of the invention provides an isolated monoclonal antibody, or an antigen-binding fragment thereof, which competes with any of the isolated monoclonal antibody or antigen-binding fragment thereof described herein for binding to hCXCR5, or for binding to the epitope bound by HFB2-4 or its Hz variants.

In some embodiment, the monoclonal antibodies of the invention or antigen-binding fragments thereof are human-mouse chimeric antibodies, humanized antibodies, human antibodies, CDR-grafted antibodies, or resurfaced antibodies.

In some embodiments, the antigen-binding fragment thereof is an Fab, Fab′, F(ab′)₂, F_{d}, single chain Fv or scFv, disulfide linked Fᵥ, V-NAR domain, IgNar, intrabody, IgGΔCH₂, minibody, F(ab′)₃, tetrabody, triabody, diabody, single-domain antibody, DVD-Ig, Fcab, mAb₂, (scFv)₂, or scFv-Fc.

In some embodiment, the monoclonal antibodies of the invention or antigen-binding fragments thereof has an engineered Fc region that enhances ADCC, such as those described herein/above, including the double mutation hG1DE.

In certain embodiments, the monoclonal antibodies of the invention or antigen-binding fragments thereof are specific for human CXCR5, e.g., substantially do not crossreact with CXCR3, and/or substantially do not crossreact with mouse or cynomolgus monkey CXCR5.

In some embodiments, the monoclonal antibody of the invention or antigen-binding fragment thereof has a dissociation constant (K_{d}) of ≤1 μM, ≤100 nM, ≤50 nM, ≤25 nM, ≤20 nM, ≤15 nM, ≤10 nM, ≤5 nM, ≤2 nM, ≤1 nM, ≤0.1 nM, ≤0.01 nM, or ≤0.001 nM (e.g. 10⁻⁸ M or less, e.g. from 10⁻⁸ M to 10⁻¹³ M, e.g., from 10⁻⁹ M to 10⁻¹³ M) for hCXCR5.

In certain embodiment, the monoclonal antibodies of the invention or antigen-binding fragments thereof bind to the epitope bound by HFB2-4, or its Hz variants, such as Hz9, Hz12, Hz14, Hz15, Hz37, Hz38, Hz39, Hz40, Hz41, and Hz42.

In certain embodiments, the monoclonal antibodies of the invention or antigen-binding fragments thereof binds hCXCR5 expressed on cells with an apparent affinity of an EC50 of about 0.4 to 4 nM, such as about 0.9 nM or about 1.2 nM.

In certain embodiments, the monoclonal antibodies of the invention or antigen-binding fragments thereof antagonizes CXCR5-CXCL13 signaling in a cAMP reporter assay with an EC50 of about 0.5 to 3.5 nM, such as about 0.49 nM.

In certain embodiments, the monoclonal antibodies of the invention or antigen-binding fragments thereof exhibits ADCC activity against cells expressing hCXCR5 with an EC50 of less than 0.1 nM, e.g., 0.001 to 0.1 nM, such as about 0.002 nM.

In certain embodiments, the monoclonal antibodies of the invention or antigen-binding fragments thereof mediates NK-cells killing of human B cells expressing hCXCR5 with an EC50 of less than 0.1 fM., or about 0.3 aM to 0.8 pM

In certain embodiments, the monoclonal antibodies of the invention or antigen-binding fragments thereof binds hCXCR5 but does not detectably bind human chemokine receptors CCR1, CCR2, CCR3, CCR4, CCR5, CCR6, CCR7, CCR8, CCR9, CCR10, CMKLR1, CXCR3R1, CXCR1, CXCR2, CXCR3, CXCR4, CXCR6, CXCR7, and XCR1.

In certain embodiments, the monoclonal antibodies of the invention or antigen-binding fragments thereof depletes B cells in the peripheral blood, optionally reversibly depletes B cells in the peripheral blood.

In certain embodiments, the monoclonal antibodies of the invention or antigen-binding fragments thereof depletes Tfh-like cells in the peripheral blood, and/or the spleen.

In certain embodiments, the monoclonal antibodies of the invention or antigen-binding fragments thereof inhibits binding of CXCR5 to CXCL13.

In certain embodiments, the monoclonal antibodies of the invention or antigen-binding fragments thereof inhibits CXCL13 inhibition of cAMP production in a cell otherwise triggered by forskolin leading to an increase in cAMP level compared to the level of cAMP in the absence of the antibody, or antigen-binding fragment thereof (e.g., inhibits CXCL13 inhibition of cAMP production with an EC50 of about 0.4 to 3.5 nM, such as about 0.49 nM). Optionally, the maximal inhibition of CXCL13 inhibition is at least about 60%, 70%, or 80%.

In certain embodiments, the monoclonal antibodies of the invention or antigen-binding fragments thereof triggers ADCC of CXCR5-expressing cells in human donor peripheral blood mononuclear cells (PBMCs).

In certain embodiments, the monoclonal antibodies of the invention or antigen-binding fragments thereof specifically binds hCXCR5 and competes with any of the antibody, or antigen-binding fragment thereof, of the invention.

TABLE A
CDR, framework (FR), variable heavy chain and variable light chain
of exemplary anti-human CXCR5 antibodies

| Antibody | | Sequence | SEQ ID NO: |
|---|---|---|---|
| HFB2-1 | VH CDR1 | GFTFSNYW | 1 |
| | VH CDR2 | IRLKSDNYAT | 2 |
| | VH CDR3 | TPPNFDY | 3 |
| | VH FR1 | EVKLEESGGGLVQPGGSMKLSCVAS | 4 |
| | VH FR2 | MNWVRQSPEKGLEWVAQ | 5 |
| | VH FR3 | HYTESVKGRFTISRDDSKSSVYLOMNNLRAEDTGIYY | 6 |
| | | C | |
| | VH FR4 | WGQGTTLTVSS | 7 |
| | VH | EVKLEESGGGLVQPGGSMKLSCVASGFTFSNYWMNWV | 8 |
| | | RQSPEKGLEWVAQIRLKSDNYATHYTESVKGRFTISR | |
| | | DDSKSSVYLQMNNLRAEDTGIYYCTPPNFDYWGQGTT | |
| | | LTVSS | |
| | VL CDR1 | TSLLHRSGKHK | 9 |
| | VL CDR2 | YVS | 10 |
| | VL CDR3 | MQSLEFPLT | 11 |
| | VL FR1 | DIVMTQAAPSVTVTPGESVTISCRST | 12 |
| | VL FR2 | FYWFLQRPGOSPQLLIY | 13 |
| | VL FR3 | NLASGVPDRFSGSGSGTDFTLRISRVEAEDFGVYYC | 14 |
| | VL FR4 | FGTGTKLEIK | 15 |
| | VL | DIVMTQAAPSVTVTPGESVTISCRSTTSLLHRSGKHK | 16 |
| | | FYWFLQRPGQSPQLLIYYVSNLASGVPDRFSGSGSGT | |
| | | DFTLRISRVEAEDFGVYYCMQSLEFPLTFGTGTKLEI | |
| | | K | |
| HFB2-2 | VH CDR1 | GFTFNTNA | 17 |
| | VH CDR2 | IRSKSNNYAT | 18 |
| | VH CDR3 | VSWDPFAY | 19 |
| | VH FR1 | EVQLVETGGGLVQPKGSLKLSCAAS | 20 |
| | VH FR2 | MNWVRQAPGKGLEWVAR | 21 |
| | VH FR3 | HYVDSVKDRFTISRDDSQSMLYLQMNNLKTEDTAMYY | 22 |
| | | C | |
| | VH FR4 | WGQGTLVTVSA | 23 |
| | VH | EVQLVETGGGLVQPKGSLKLSCAASGFTFNTNAMNWV | 24 |
| | | RQAPGKGLEWVARIRSKSNNYATHYVDSVKDRFTISR | |
| | | DDSQSMLYLQMNNLKTEDTAMYYCVSWDPFAYWGQGT | |
| | | LVTVSA | |
| | VL CDR1 | QSVLYSSNQKNY | 25 |
| | VL CDR2 | WAS | 26 |
| | VL CDR3 | HQYLSSWT | 27 |
| | VL FR1 | NIMMTQSPSSLAVSAGEKVTMSCKSS | 28 |
| | VL FR2 | LAWYQQKPGOSPKLLIY | 29 |
| | VL FR3 | TRASGVPDRFTGSGSGTDFTLTISSVOTEDLAVYYC | 30 |
| | VL FR4 | FGGGAKLEIK | 31 |
| | VL | NIMMTQSPSSLAVSAGEKVTMSCKSSQSVLYSSNQKN | 32 |
| | | YLAWYQQKPGQSPKLLIYWASTRASGVPDRFTGSGSG | |
| | | TDFTLTISSVQTEDLAVYYCHQYLSSWTFGGGAKLEI | |
| | | K | |
| HFB2-3 | VH CDR1 | GFSLTSYG | 33 |
| | VH CDR2 | TWSGGRT | 34 |
| | VH CDR3 | ARGGNY | 35 |
| | VH FR1 | QVQLKQSGPGLVOPSQSLSITCTVS | 36 |
| | VH FR2 | VHWVRQSPGKGLEWLGV | 37 |
| | VH FR3 | DYNAAFISRLSINKDNSKSQVFFKMNSLQVNDTAIYY | 38 |
| | | C | |
| | VH FR4 | WGQGTSVTVSS | 39 |
| | VH | QVQLKQSGPGLVQPSQSLSITCTVSGFSLTSYGVHWV | 40 |
| | | RQSPGKGLEWLGVTWSGGRTDYNAAFISRLSINKDNS | |
| | | KSQVFFKMNSLQVNDTAIYYCARGGNYWGQGTSVTVS | |
| | | S | |
| | VL CDR1 | ESVDNYGISF | 41 |
| | VL CDR2 | AAS | 42 |
| | VL CDR3 | QQSKEVPWT | 43 |
| | VL FR1 | DIVLTQSPASLTVSLGQRATISCRAS | 44 |
| | VL FR2 | MNWFQQKPGQPPKLLIY | 45 |
| | VL FR3 | NQGSGVPARFSGSGSGTDFSLNIHPMEEDDTAMYFC | 46 |
| | VL FR4 | FGGGTKLEIK | 47 |
| | VL | DIVLTQSPASLTVSLGQRATISCRASESVDNYGISFM | 48 |
| | | NWFQQKPGQPPKLLIYAASNQGSGVPARFSGSGSGTD | |
| | | QVQLKESGPGLVAPSQSLSITCTVSGFSLTSYGVSWV | |
| | | RQPPGKGLEWLGVIWGDGSTNYHSTLISRLSISKDNS | |
| | | KSQVFLKLNSLQTDDTATYYCARVAYWGQGTLVTVSA | |
| | | FSLNIHPMEEDDTAMYFCQQSKEVPWTFGGGTKLEIK | |
| HFB2-4 | VH CDR1 | GFSLTSYG | 33 |
| | VH CDR2 | IWGDGST | 49 |
| | VH CDR3 | ARVVY | 51 |
| | VH FR1 | QVQLKESGPGLVAPSQSLSITCTVS | 52 |
| | VH FR2 | VSWVRQPPGKGLEWLGV | 53 |
| | VH FR3 | NYHSGLISRLSISKDNSKSQVFLKLNSLOSDDTATYY | 54 |
| | | C | |
| | VH FR4 | WGQGTLVTVSA | 55 |
| | VH | QVQLKESGPGLVAPSQSLSITCTVSGFSLTSYGVSWV | 56 |
| | | RQPPGKGLEWLGVIWGDGSTNYHSGLISRLSISKDNS | |
| | | KSQVFLKLNSLOSDDTATYYCARVVYWGQGTLVTVSA | |
| | VL CDR1 | KSLLHSNGKTY | 57 |
| | VL CDR2 | RMS | 58 |
| | VL CDR3 | MQHLEYPYT | 59 |
| | VL FR1 | DIVMTQAAPSVPVTPGESISISCRSS | 60 |
| | VL FR2 | LYWFLORPGOSPOLLLY | 61 |
| | VL FR3 | NLASGVPDRFSGSGSGTAFTLRISRVEAEDVGVYYC | 62 |
| | VL FR4 | FGGGTKLEIK | 47 |
| | VL | DIVMTQAAPSVPVTPGESISISCRSSKSLLHSNGKTY | 63 |
| | | LYWFLQRPGQSPQLLLYRMSNLASGVPDRFSGSGSGT | |
| | | AFTLRISRVEAEDVGVYYCMQHLEYPYTFGGGTKLEI | |
| | | K | |
| HFB2-5 | VH CDR1 | GFSLTSYG | 33 |
| | VH CDR2 | IWGDGST | 49 |
| | VH CDR3 | ARVAY | 65 |
| | VH FR1 | QVQLKESGPGLVAPSQSLSITCTVS | 52 |
| | VH FR2 | VSWVRQPPGKGLEWLGV | 53 |
| | VH FR3 | NYHSTLISRLSISKDNSKSQVFLKLNSLOTDDTATYY | 64 |
| | | C | |
| | VH FR4 | WGQGTLVTVSA | 55 |
| | VH | QVQLKESGPGLVAPSQSLSITCTVSGFSLTSYGVSWV | 65 |
| | | RQPPGKGLEWLGVIWGDGSTNYHSTLISRLSISKDNS | |
| | | KSQVFLKLNSLQTDDTATYYCARVAYWGQGTLVTVSA | |
| | VL CDR1 | KSLLHSNGKTY | 57 |
| | VL CDR2 | RMS | 58 |
| | VL CDR3 | MQHLEYPYT | 59 |
| | VL FR1 | DIVMTQAAPSIPVTPGESVSISCRSS | 66 |
| | VL FR2 | LYWFLQRPGQSPQLLIY | 67 |
| | VL FR3 | NLASGVPDRFSGSGSGTAFTLRISRVEAEDVGVYYC | 62 |
| | VL FR4 | FGGGTKLEIK | 47 |
| | VL | DIVMTQAAPSIPVTPGESVSISCRSSKSLLHSNGKTY | 68 |
| | | LYWFLQRPGQSPQLLIYRMSNLASGVPDRFSGSGSGT | |
| | | AFTLRISRVEAEDVGVYYCMQHLEYPYTFGGGTKLEI | |
| | | K | |
| HFB2-6 | VH CDR1 | GYTFTDYT | 69 |
| | VH CDR2 | IYPGSGNI | 70 |
| | VH CDR3 | ARGLRREFAY | 71 |
| | VH FR1 | QIQLQQSGPELVEPGASVKLSCKAS | 72 |
| | VH FR2 | IHWVKQSPGQGLEWIGW | 73 |
| | VH FR3 | KYNDKFKGKATMTADKSSSTAYMQLSSLTSEDSAVYF | 74 |
| | | C | |
| | VH FR4 | WGQGTLVTVSA | 55 |
| | VH | QIQLQQSGPELVEPGASVKLSCKASGYTFTDYTIHWV | 75 |
| | | KQSPGQGLEWIGWIYPGSGNIKYNDKFKGKATMTADK | |
| | | SSSTAYMQLSSLTSEDSAVYFCARGLRREFAYWGQGT | |
| | | LVTVSA | |
| | VL CDR1 | KSLLHSNGITY | 76 |
| | VL CDR2 | QMS | 77 |
| | VL CDR3 | AQSLELPLT | 78 |
| | VL FR1 | DIVMTQAAFSNPVTLGTSASISCRSS | 79 |
| | VL FR2 | LYWYLQKPGQSPOLLIY | 80 |
| | VL FR3 | NLASGVPDRESSSGSGTDFTLRISRVEAEDVGVYYC | 81 |
| | VL FR4 | FGAGTKLELK | 82 |
| | VL | DIVMTQAAFSNPVTLGTSASISCRSSKSLLHSNGITY | 83 |
| | | LYWYLOKPGQSPQLLIYQMSNLASGVPDRESSSGSGT | |
| | | DFTLRISRVEAEDVGVYYCAQSLELPLTFGAGTKLEL | |
| | | K | |

In some embodiments, the anti-human CXCR5 antibody or antigen-binding fragment thereof of the invention comprises at least one, two, or three (e.g., all three) corresponding VH CDRs of any one of the antibodies listed in Table A and Table B.

In some embodiments, the anti-human CXCR5 antibody or antigen-binding fragment thereof of the invention comprises at least one, two, or three (e.g., all three) corresponding VL CDRs of any one of the antibodies listed in Table A and Table C.

In some embodiments, the monoclonal antibodies of the invention or antigen-binding fragments thereof comprises the VH CDR1, VH CDR2 and VH CDR3 sequences comprising the amino acid sequence of SEQ ID NOs: 1, 2, and 3, respectively, and the VL CDR1, VL CDR2 and VL CDR3 sequences comprising the amino acid sequence of SEQ ID NOs: 9, 10, and 11 respectively.

In some embodiments, the monoclonal antibodies of the invention or antigen-binding fragments thereof comprises the VH CDR1, VH CDR2 and VH CDR3 sequences comprising the amino acid sequence of SEQ ID NOs: 17, 18, and 19, respectively, and the VL CDR1, VL CDR2 and VL CDR3 sequences comprising the amino acid sequence of SEQ ID NOs: 25, 26, and 27 respectively.

In some embodiments, the monoclonal antibodies of the invention or antigen-binding fragments thereof comprises the VH CDR1, VH CDR2 and VH CDR3 sequences comprising the amino acid sequence of SEQ ID NOs: 33, 34, and 35, respectively, and the VL CDR1, VL CDR2 and VL CDR3 sequences comprising the amino acid sequence of SEQ ID NOs: 41, 42, and 43 respectively.

In some embodiments, the monoclonal antibodies of the invention or antigen-binding fragments thereof comprises the VH CDR1, VH CDR2 and VH CDR3 sequences comprising the amino acid sequence of SEQ ID NOs: 33, 49, and 51, respectively, and the VL CDR1, VL CDR2 and VL CDR3 sequences comprising the amino acid sequence of SEQ ID NOs: 57, 58, and 59 respectively.

In some embodiments, the monoclonal antibodies of the invention or antigen-binding fragments thereof comprises the VH CDR1, VH CDR2 and VH CDR3 sequences comprising the amino acid sequence of SEQ ID NOs: 33, 49, and 65, respectively, and the VL CDR1, VL CDR2 and VL CDR3 sequences comprising the amino acid sequence of SEQ ID NOs: 57, 58, and 59 respectively.

In some embodiments, the monoclonal antibodies of the invention or antigen-binding fragments thereof comprises the VH CDR1, VH CDR2 and VH CDR3 sequences comprising the amino acid sequence of SEQ ID NOs: 69, 70, and 71, respectively, and the VL CDR1, VL CDR2 and VL CDR3 sequences comprising the amino acid sequence of SEQ ID NOs: 76, 77, and 78 respectively.

In some embodiments, the monoclonal antibodies of the invention or antigen-binding fragments thereof comprises the VH CDR1, VH CDR2 and VH CDR3 sequences comprising the amino acid sequence of SEQ ID NOs: 33, 49, and 51, respectively, and the VL CDR1, VL CDR2 and VL CDR3 sequences comprising the amino acid sequences of SEQ ID NOs: 149, 150 and 151, respectively.

In some embodiments, the monoclonal antibodies of the invention or antigen-binding fragments thereof comprises the VH CDR1, VH CDR2 and VH CDR3 sequences comprising the amino acid sequence of SEQ ID NOs: 114, 115, and 116, respectively, and the VL CDR1, VL CDR2 and VL CDR3 sequences comprising the amino acid sequences of SEQ ID NOs: 120, 121, and 122 respectively.

In certain embodiments, the monoclonal antibodies of the invention or antigen-binding fragments thereof comprises up to 1, 2, 3, 4, or 5 (e.g., up to 1, 2, or 3) amino acid residue changes (e.g., deletions, insertions or substitutions (e.g., conservative substitutions)) in any one or more of VH CDR1, VH CDR2 and VH CDR3 of the antibody of the invention; and/or 1, 2, 3, 4, or 5 (e.g., up to 1, 2, or 3) amino acid residue changes (e.g., deletions, insertions or substitutions (e.g., conservative substitutions)) in any one or more of VL CDR1, VH CDR2 and VH CDR3 of the antibody of the invention.

Anti-human CXCR5 antibodies or antigen-binding fragments thereof according to the present disclosure may be prepared using any of the framework region (FR) of amino acid sequences as described in Table A, or sequences substantially identical (e.g., having at least about 80%, 85%, 90%, 92%, 95%, 97%, 98%, or 99% sequence identity) to the FR amino acid sequences as described in Table A. In certain embodiments, the heavy chain FR regions are all from the same exemplified antibodies herein. In certain embodiments, the light chain FR regions are all from the same exemplified antibodies herein. In certain embodiments, both the heavy and light chain FR regions are all from the same exemplified antibodies herein.

In some embodiments, the monoclonal antibody or antigen-binding fragment thereof of the invention has a heavy chain variable region (VH) comprising one, two, three, or all (i.e., four) of a heavy chain framework region 1 (VH FR1), a heavy chain framework region 2 (VH FR2), a heavy chain framework region 3 (VH FR3), and/or a heavy chain framework region 4 (VH FR4) of the corresponding heavy chain framework regions of any one of the antibodies listed in Table A, or a VH FR1, VH FR2, VH FR3 and/or VH FR4 comprising sequences substantially identical (e.g., having at least about 80%, 85%, 90%, 92%, 95%, 97%, 98%, or 99% sequence identity) to the corresponding VH FR amino acid sequences of any one of the antibodies as described in Table A.

In some embodiments, the monoclonal antibody or antigen-binding fragment thereof of the invention comprises a VH FR1 of SEQ ID NO: 4, 20, 36, 52 or 72, or an amino acid sequence substantially identical (e.g., having at least about 80%, 85%, 90%, 92%, 95%, 97%, 98%, or 99% sequence identity) to SEQ ID NO: 4, 20, 36, 52 or 72.

In some embodiments, the monoclonal antibody or antigen-binding fragment thereof of the invention comprises a VH FR2 of SEQ ID NO: 5, 21, 37, 53 or 73, or an amino acid sequence substantially identical (e.g., having at least about 80%, 85%, 90%, 92%, 95%, 97%, 98%, or 99% sequence identity) to SEQ ID NO: SEQ ID NO: 5, 21, 37, 53 or 73.

In some embodiments, the monoclonal antibody or antigen-binding fragment thereof of the invention comprises a VH FR3 of SEQ ID NO: 6, 22, 38, 54, 64 or 74, or an amino acid sequence substantially identical (e.g., having at least about 80%, 85%, 90%, 92%, 95%, 97%, 98%, or 99% sequence identity) to SEQ ID NO: 6, 22, 38, 54, 64 or 74.

In some embodiments, the monoclonal antibody or antigen-binding fragment thereof of the invention comprises a VH FR4 of SEQ ID NO: 7, 23, 39 or 55, or an amino acid sequence substantially identical (e.g., having at least about 80%, 85%, 90%, 92%, 95%, 97%, 98%, or 99% sequence identity) to SEQ ID NO: 7, 23, 39 or 55.

In some embodiments, the monoclonal antibody or antigen-binding fragment thereof of the invention has a light chain variable region (VL) comprising one, two, three, or all (i.e., four) of a heavy chain framework region 1 (VL FR1), a heavy chain framework region 2 (VL FR2), a heavy chain framework region 3 (VL FR3), and/or a heavy chain framework region 4 (VL FR4) of the corresponding heavy chain framework regions of any one of the antibodies listed in Table A, or a VL FR1, VL FR2, VL FR3 and/or VL FR4 comprising sequences substantially identical (e.g., having at least about 80%, 85%, 90%, 92%, 95%, 97%, 98%, or 99% sequence identity) to the corresponding VL FR amino acid sequences of any one of the antibodies as described in Table A.

In some embodiments, the monoclonal antibody or antigen-binding fragment thereof of the invention comprises a VL FR1 of SEQ ID NO: 12, 28, 44, 60, 66 or 79, or an amino acid sequence substantially identical (e.g., having at least about 80%, 85%, 90%, 92%, 95%, 97%, 98%, or 99% sequence identity) to SEQ ID NO: 12, 28, 44, 60, 66 or 79.

In some embodiments, the monoclonal antibody or antigen-binding fragment thereof of the invention comprises a VL FR2 of SEQ ID NO: 13, 29, 45, 61, 67 or 80, or an amino acid sequence substantially identical (e.g., having at least about 80%, 85%, 90%, 92%, 95%, 97%, 98%, or 99% sequence identity) to SEQ ID NO: 13, 29, 45, 61, 67 or 80.

In some embodiments, the monoclonal antibody or antigen-binding fragment thereof of the invention comprises a VL FR3 of SEQ ID NO: 14, 30, 46, 62 or 81, or an amino acid sequence substantially identical (e.g., having at least about 80%, 85%, 90%, 92%, 95%, 97%, 98%, or 99% sequence identity) to SEQ ID NO: 14, 30, 46, 62 or 81.

In some embodiments, the monoclonal antibody or antigen-binding fragment thereof of the invention comprises a VL FR4 of SEQ ID NO: 15, 31, 47 or 82, or an amino acid sequence substantially identical (e.g., having at least about 80%, 85%, 90%, 92%, 95%, 97%, 98%, or 99% sequence identity) to SEQ ID NO: 15, 31, 47 or 82.

In certain embodiments, the monoclonal antibodies of the invention or antigen-binding fragments thereof comprises a VL framework sequence and a VH framework sequence, and wherein one or both of the VL framework sequence or VH framework sequence is at least 90%, 91%, 92%, 93%, 94%, 95%, 96%, 97%, 98% or 99% identical to the human germline sequence form which it was derived and wherein the human germline VL sequence from which the VL framework sequence is derived is selected from the group consisting of V1-22, Vλ consensus, Vλ1 consensus, Vλ3 consensus, Vκ consensus, Vκ1 consensus, Vκ2 consensus and Vκ3, and wherein the human germline VH sequence from which the VH framework sequence is derived is selected from the group consisting of VH3, VH5, VH1 and VH4.

In certain embodiments, the monoclonal antibodies of the invention or antigen-binding fragments thereof comprises a VL framework sequence and a VH framework sequence, and wherein one or both of the VL framework sequence and/or VH framework sequence is at least 66%, 76%, 80%, 90%, 91%, 92%, 93%, 94%, 95%, 96%, 97%, 98%, or 99% identical to the human germline sequence from which it was derived.

In certain embodiments, the monoclonal antibodies of the invention or antigen-binding fragments thereof comprises a VL framework sequence and a VH framework sequence, and wherein one or both of the VL framework sequence or VH framework sequence is identical to the human germline sequence from which it was derived.

In certain embodiments, the monoclonal antibodies of the invention or antigen-binding fragments thereof is afucosylated.

In certain embodiments, the monoclonal antibodies of the invention or antigen-binding fragments thereof exhibits enhanced ADCC.

In certain embodiments, the monoclonal antibodies of the invention or antigen-binding fragments thereof comprises a framework VH sequence derived from a human VH3 germline sequence.

In certain embodiments, the monoclonal antibodies of the invention or antigen-binding fragments thereof comprising an Fc domain, such as the Fc domain of an IgA (for example IgA1 or IgA2), IgD, IgE, IgM, or IgG (for example IgG1, IgG2, IgG3, or IgG4). In certain embodiments, the Fc domain is the Fc domain of an IgG, such as that of (human) IgG1, IgG2, IgG3, or IgG4.

In certain embodiments, the monoclonal antibodies of the invention or antigen-binding fragments thereof, wherein the antibody or antigen-binding fragment is an Fc fusion protein, a monobody, a maxibody, a bifunctional antibody, an scFab, an scFv, a peptibody.

In certain embodiments, the monoclonal antibodies of the invention or antigen-binding fragments thereof binds human CXCR5 with a K_{D} about or less than a value selected from the group consisting of about 10 nM, 5 nM, 2 nM, 1 nM, 900 pM, 800 pM, 700 pM, 600 pM, 500 pM, 400 pM, 300 pM, 250 pM, 200 pM, 150 pM, 100 pM, 50 pM, 40 pM, 30 pM, 25 pM, 20 pM, 15 pM, 10 pM, 5 pM, and 1 pM.

In certain embodiments, the monoclonal antibodies of the invention or antigen-binding fragments thereof has a predicted half-life in human of from about one (1) day, to seven (7) days.

In some embodiments, the monoclonal antibodies of the invention or antigen-binding fragments thereof, including humanized monoclonal antibodies or antigen-binding fragments thereof, have good developability profile, including being stable under high temperature (e.g., 25° C. or 40° C.), low pH conditions (e.g., pH3.5 around room temperature), and/or following several rounds of freeze/thaw cycles.

In certain embodiments, the monoclonal antibodies of the invention or antigen-binding fragments thereof, including humanized monoclonal antibodies or antigen-binding fragments thereof, include one or more point mutations of in amino acid sequences that are designed to improve developability of the antibody. For example, Raybould et al. (Five computational developability guidelines for therapeutic antibody profiling, PNAS 116(10): 4025-4030, 2019) described Therapeutic Antibody Profiler (TAP), a computational tool that builds downloadable homology models of variable domain sequences, tests them against five developability guidelines, and reports potential sequence liabilities and canonical forms. The authors further provide TAP as freely available at opig.stats.ox.ac.uk/webapps/sabdab-sabpred/TAP.php.

There are many barriers to therapeutic mAb development, besides achieving the desired affinity to the antigen. These include intrinsic immunogenicity, chemical and conformational instability, self-association, high viscosity, polyspecificity, and poor expression. For example, high levels of hydrophobicity, particularly in the highly variable complementarity-determining regions (CDRs), have repeatedly been implicated in aggregation, viscosity, and polyspecificity. Asymmetry in the net charge of the heavy- and light-chain variable domains is also correlated with self-association and viscosity at high concentrations. Patches of positive and negative charge in the CDRs are linked to high rates of clearance and poor expression levels. Product heterogeneity (e.g., through oxidation, isomerization, or glycosylation) often results from specific sequence motifs liable to post- or co-translational modification. Computational tools are available to facilitate the identification of sequence liabilities. Warszawski et al. (Optimizing antibody affinity and stability by the automated design of the variable light-heavy chain interfaces. PLoS Comput Biol 15(8): e1007207. https://doi.org/10.1371/journal.pcbi.1007207) also described methods of optimizing antibody affinity and stability by an automated design of the variable light-heave chain interfaces. Additional methods are available to identify potential developability issues of a candidate antibody, and in preferred embodiments of this invention, one or more point mutations can be introduced, via conventional methods, to the candidate antibody to address such issues to lead to an optimized therapeutic antibody of the invention.

### 7. Humanized Antibodies

In some embodiments, the antibody of the invention is a humanized antibody. Humanized antibodies are useful as therapeutic molecules because humanized antibodies reduce or eliminate the human immune response to non-human antibodies (such as the human anti-mouse antibody (HAMA) response), which can result in an immune response to an antibody therapeutic, and decreased effectiveness of the therapeutic.

An antibody may be humanized by any standard method. Non-limiting exemplary methods of humanization include methods described, e.g., in U.S. Pat. Nos. 5,530,101; 5,585,089; 5,693,761; 5,693,762; 6,180,370; Jones et al., Nature 321:522-525 (1986); Riechmann et al, Nature 332: 323-27 (1988); Verhoeyen et al, Science 239: 1534-36 (1988); and U.S. Publication No. US 2009/0136500. All incorporated by reference.

A humanized antibody is an antibody in which at least one amino acid in a framework region of a non-human variable region has been replaced with the amino acid from the corresponding location in a human framework region. In some embodiments, at least two, at least three, at least four, at least five, at least six, at least seven, at least eight, at least nine, at least 10, at least 11, at least 12, at least 15, or at least 20 amino acids in the framework regions of a non-human variable region are replaced with an amino acid from one or more corresponding locations in one or more human framework regions.

In some embodiments, some of the corresponding human amino acids used for substitution are from the framework regions of different human immunoglobulin genes. That is, in some such embodiments, one or more of the non-human amino acids may be replaced with corresponding amino acids from a human framework region of a first human antibody or encoded by a first human immunoglobulin gene, one or more of the non-human amino acids may be replaced with corresponding amino acids from a human framework region of a second human antibody or encoded by a second human immunoglobulin gene, one or more of the non-human amino acids may be replaced with corresponding amino acids from a human framework region of a third human antibody or encoded by a third human immunoglobulin gene, etc. Further, in some embodiments, all of the corresponding human amino acids being used for substitution in a single framework region, for example, FR2, need not be from the same human framework. In some embodiments, however, all of the corresponding human amino acids being used for substitution are from the same human antibody or encoded by the same human immunoglobulin gene.

In some embodiments, an antibody is humanized by replacing one or more entire framework regions with corresponding human framework regions. In some embodiments, a human framework region is selected that has the highest level of homology to the non-human framework region being replaced. In some embodiments, such a humanized antibody is a CDR-grafted antibody.

In some embodiments, following CDR-grafting, one or more framework amino acids are changed back to the corresponding amino acid in a mouse framework region. Such “back mutations” are made, in some embodiments, to retain one or more mouse framework amino acids that appear to contribute to the structure of one or more of the CDRs and/or that may be involved in antigen contacts and/or appear to be involved in the overall structural integrity of the antibody. In some embodiments, ten or fewer, nine or fewer, eight or fewer, seven or fewer, six or fewer, five or fewer, four or fewer, three or fewer, two or fewer, one, or zero back mutations are made to the framework regions of an antibody following CDR grafting.

TABLE B
Exemplary humanized VH sequences derived from HFB2-4

| | | | SEQ ID |
|---|---|---|---|
| VH sequence | | Sequence | NO: |
| HFB2-4hz-VH1 | VH CDR1 | GFSLTSYG | 33 |
| | VH CDR2 | IWGDGST | 49 |
| | VH CDR3 | ARVVY | 51 |
| | VH FR1 | QVQLVOSGAEVKKPGSSVKVSCKAS | 84 |
| | VH FR2 | VSWVRQAPGQGLEWMGV | 85 |
| | VH FR3 | NYAQKFQGRVTITKDESTSTAYMELSSLRSEDTAVYY | 86 |
| | | C | |
| | VH FR4 | WGQGTLVTVSS | 87 |
| | VH | QVQLVQSGAEVKKPGSSVKVSCKASGFSLTSYGVSWV | 88 |
| | | RQAPGQGLEWMGVIWGDGSTNYAQKFQGRVTITKDES | |
| | | TSTAYMELSSLRSEDTAVYYCARVVYWGQGTLVTVSS | |
| HFB2-4hz-VH2 | VH CDR1 | GFSLTSYG | 33 |
| | VH CDR2 | IWGDGST | 49 |
| | VH CDR3 | ARVVY | 51 |
| | VH FR1 | EVQLVESGGGLVQPGGSLRLSCAAS | 89 |
| | VH FR2 | VSWVRQAPGKGLEWVGV | 90 |
| | VH FR3 | NYAASVKGRFTISRDDSKNSVYLQMNSLKTEDTAVYY | 91 |
| | | C | |
| | VH FR4 | WGQGTLVTVSS | 87 |
| | VH | EVQLVESGGGLVQPGGSLRLSCAASGFSLTSYGVSWV | 92 |
| | | RQAPGKGLEWVGVIWGDGSTNYAASVKGRFTISRDDS | |
| | | KNSVYLQMNSLKTEDTAVYYCARVVYWGQGTLVTVSS | |
| HFB2-4hz-VH3 | VH CDR1 | GFSLTSYG | 33 |
| | VH CDR2 | IWGDGST | 49 |
| | VH CDR3 | ARVVY | 51 |
| | VH FR1 | QVQLQESGPGLVKPSETLSLTCTVS | 93 |
| | VH FR2 | VSWVRQPPGKGLEWIGV | 94 |
| | VH FR3 | NYNPSLKSRVTISKDTSKNQVSLKLSSVTAADTAVYY | 95 |
| | | C | |
| | VH FR4 | WGQGTLVTVSS | 87 |
| | VH | QVQLQESGPGLVKPSETLSLTCTVSGFSLTSYGVSWV | 96 |
| | | RQPPGKGLEWIGVIWGDGSTNYNPSLKSRVTISKDTS | |
| | | KNQVSLKLSSVTAADTAVYYCARVVYWGQGTLVTVSS | |
| HFB2-4hz-VH6 | VH CDR1 | GFSLTSYG | 33 |
| | VH CDR2 | IWGDGST | 49 |
| | VH CDR3 | ARVVY | 51 |
| | VH FR1 | QVQLQESGPGLVKPSGTLSLTCAVS | 138 |
| | VH FR2 | VSWVRQPPGKGLEWIGV | 94 |
| | VH FR3 | NYNPSLKSRLTISKDKSKNQVSLKLSSVTAADTAVYY | 139 |
| | | C | |
| | VH FR4 | WGQGTLVTVSS | 87 |
| | VH | QVQLQESGPGLVKPSGTLSLTCAVSGFSLTSYGVSWV | 140 |
| | | RQPPGKGLEWIGVIWGDGSTNYNPSLKSRLTISKDKS | |
| | | KNQVSLKLSSVTAADTAVYYCARVVYWGQGTLVTVSS | |
| HFB2-4hz-VH8 | VH CDR1 | GFSLTSYG | 33 |
| | VH CDR2 | IWGDGST | 49 |
| | VH CDR3 | ARVVY | 51 |
| | VH FR1 | QVQLVESGGGVVQPGRSLRLSCAVS | 141 |
| | VH FR2 | VSWVRQAPGKGLEWLGV | 142 |
| | VH FR3 | NYHSGLISRLTISKDNSKNTVYLQMNSLRAEDTAVYY | 143 |
| | | C | |
| | VH FR4 | WGQGTLVTVSS | 87 |
| | VH | QVQLVESGGGVVQPGRSLRLSCAVSGFSLTSYGVSWV | 144 |
| | | RQAPGKGLEWLGVIWGDGSTNYHSGLISRLTISKDNS | |
| | | KNTVYLQMNSLRAEDTAVYYCARVVYWGQGTLVTVSS | |
| HFB2-4hz-VH10 | VH CDR1 | GFSLTSYG | 33 |
| | VH CDR2 | IWGDGST | 49 |
| | VH CDR3 | ARVVY | 51 |
| | VH FR1 | QVQLVOSGAEVKKPGASVKVSCKAS | 132 |
| | VH FR2 | VSWVRQAPGQGLEWMGV | 85 |
| | VH FR3 | NYAQKLQGRVTMTKDTSTSTAYMELRSLRSDDTAVYY | 133 |
| | | C | |
| | VH FR4 | WGQGTLVTVSS | 87 |
| | VH | QVQLVQSGAEVKKPGASVKVSCKASGFSLTSYGVSWV | 113 |
| | | RQAPGQGLEWMGVIWGDGSTNYAQKLQGRVTMTKDTS | |
| | | TSTAYMELRSLRSDDTAVYYCARVVYWGQGTLVTVSS | |

TABLE C
Exemplary humanized VL sequences derived from HFB2-4

| | | | SEQ |
|---|---|---|---|
| VL sequence | | Sequence | ID NO: |
| HFB2-4hz-VL1 | VL CDR1 | KSLLHSNGKTY | 57 |
| | VL CDR2 | RMS | 58 |
| | VL CDR3 | MQHLEYPYT | 59 |
| | VL FR1 | DIVMTQSPLSLPVTPGEPASISCRSS | 97 |
| | VL FR2 | LYWFLQKPGQSPOLLIY | 98 |
| | VL FR3 | NLASGVPDRFSGSGSGTDFTLKISRVEAEDVGVYYC | 99 |
| | VL FR4 | FGGGTKVEIK | 100 |
| | VL | DIVMTQSPLSLPVTPGEPASISCRSSKSLLHSNGKTYLY | 101 |
| | | WFLOKPGQSPQLLIYRMSNLASGVPDRFSGSGSGTDFTL | |
| | | KISRVEAEDVGVYYCMQHLEYPYTFGGGTKVEIK | |
| HFB2-4hz-VL2 | VL CDR1 | KSLLHSNGKTY | 57 |
| | VL CDR2 | RMS | 58 |
| | VL CDR3 | MQHLEYPYT | 59 |
| | VL FR1 | DIVMTQSPLSLPVTPGEPASISCRSS | 97 |
| | VL FR2 | LYWFLQKPGOSPOLLLY | 102 |
| | VL FR3 | NLASGVPDRFSGSGSGTDFTLKISRVEAEDVGVYYC | 99 |
| | VL FR4 | FGGGTKVEIK | 100 |
| | VL | DIVMTQSPLSLPVTPGEPASISCRSSKSLLHSNGKTYLY | 137 |
| | | WFLQKPGOSPQLLLYRMSNLASGVPDRFSGSGSGTDFTL | |
| | | KISRVEAEDVGVYYCMQHLEYPYTFGGGTKVEIK | |
| HFB2-4hz-VL3 | VL CDR1 | KSLLHSNGKTY | 57 |
| | VL CDR2 | RMS | 58 |
| | VL CDR3 | MQHLEYPYT | 59 |
| | VL FR1 | DIQMTQSPSSLSASVGDRVTITCRSS | 103 |
| | VL FR2 | LYWFQQKPGKAPKLLLY | 104 |
| | VL FR3 | NLASGVPSRFSGSGSGTDFTLTISSLQPEDVATYYC | 105 |
| | VL FR4 | FGGGTKVEIK | 100 |
| | VL | DIQMTQSPSSLSASVGDRVTITCRSSKSLLHSNGKTYLY | 106 |
| | | WFQQKPGKAPKLLLYRMSNLASGVPSRFSGSGSGTDFTL | |
| | | TISSLQPEDVATYYCMQHLEYPYTFGGGTKVEIK | |
| HFB2-4hz-VL4 | VL CDR1 | KSLLHSNGKTY | 57 |
| | VL CDR2 | RMS | 58 |
| | VL CDR3 | MQHLEYPYT | 59 |
| | VL FR1 | DIQMTQSPSSLSASVGDRVTITCRSS | 103 |
| | VL FR2 | LYWFQQKPGKAPKLLIY | 107 |
| | VL FR3 | NLASGVP SRFSGSGSGTDFTLTISSLQPEDFATYYC | 108 |
| | VL FR4 | FGGGTKVEIK | 100 |
| | VL | DIQMTQSPSSLSASVGDRVTITCRSSKSLLHSNGKTYLY | 109 |
| | | WFQQKPGKAPKLLIYRMSNLASGVPSRFSGSGSGTDFTL | |
| | | TISSLQPEDFATYYCMQHLEYPYTFGGGTKVEIK | |
| HFB2-4hz-VL6 | VL CDR1 | KSLLHSNGKTY | 57 |
| | VL CDR2 | RMS | 58 |
| | VL CDR3 | MQHLEYPYT | 59 |
| | VL FR1 | EIVMTQSPATLSVSPGERATLSCRSS | 134 |
| | VL FR2 | LYWFQQKPGQAPRLLIY | 135 |
| | VL FR3 | NLASGIPARFSGSGSGTEFTLTISSVQSEDVAVYYC | 136 |
| | VL FR4 | FGQGTKLEIK | 131 |
| | VL | EIVMTQSPATLSVSPGERATLSCRSSKSLLHSNGKTYLY | 112 |
| | | WFQQKPGQAPRLLIYRMSNLASGIPARFSGSGSGTEFTL | |
| | | TISSVQSEDVAVYYCMQHLEYPYTFGQGTKLEIK | |
| HFB2-4hz-VL7 | VL CDR1 | KSLLHSNGKTY | 57 |
| | VL CDR2 | RMS | 58 |
| | VL CDR3 | MQHLEYPYT | 59 |
| | VL FR1 | DIVMTQSPDSLAVSLGERATINCRSS | 145 |
| | VL FR2 | LYWFQQKPGOSPKLLLY | 146 |
| | VL FR3 | NLASGVPDRFSGSGSGTDFTLTISSLQAEDVAVYYC | 147 |
| | VL FR4 | FGQGTKLEIK | 131 |
| | VL | DIVMTQSPDSLAVSLGERATINCRSSKSLLHSNGKTYLY | 148 |
| | | WFQQKPGQSPKLLLYRMSNLASGVPDRFSGSGSGTDFTL | |
| | | TISSLQAEDVAVYYCMQHLEYPYTFGQGTKLEIK | |
| HFB2-4hz- | VL CDR1 | QSLLHSNGYNY | 149 |
| VL1b | VL CDR2 | RGS | 150 |
| | VL CDR3 | MQHLQYPYT | 151 |
| | VL FR1 | DIVMTQSPLSLPVTPGEPASISCRSS | 97 |
| | VL FR2 | LYWFLOKPGQSPOLLIY | 98 |
| | VL FR3 | NRASGVPDRFSGSGSGTDFTLKISRVEAEDVGVYYC | 152 |
| | VL FR4 | FGGGTKVEIK | 100 |
| | VL | DIVMTQSPLSLPVTPGEPASISCRSSQSLLHSNGYNYLY | 153 |
| | | WFLQKPGQSPQLLIYRGSNRASGVPDRFSGSGSGTDFTL | |
| | | KISRVEAEDVGVYYCMQHLQYPYTFGGGTKVEIK | |
| HFB2-4hz- | VL CDR1 | KSLLHSNGKTY | 57 |
| VL1c | VL CDR2 | RMS | 58 |
| | VL CDR3 | MQHLEYPYT | 59 |
| | VL FR1 | DIVMTQSPLSLPVTPGEPISLSCRSS | 154 |
| | VL FR2 | LYWFLQKPGOSPQLLLY | 102 |
| | VL FR3 | NLASGVPDRFSGSGSGTDFTLKISRVEAEDVGVYYC | 99 |
| | VL FR4 | FGGGTKLEIK | 47 |
| | VL | DIVMTQSPLSLPVTPGEPISLSCRSSKSLLHSNGKTYLY | 155 |
| | | WFLOKPGQSPQLLLYRMSNLASGVPDRFSGSGSGTDFTL | |
| | | KISRVEAEDVGVYYCMQHLEYPYTFGGGTKLEIK | |

TABLE D
Exemplary humanized antibodies derived from HFB2-4

| | | | SEQ |
|---|---|---|---|
| Antibody | | Sequence | ID NO: |
| HFB2-4hG1 | VH CDR1 | GFSLTSYGVS | 114 |
| (parental) | VH CDR2 | VIWGDGSTN | 115 |
| | VH CDR3 | VVY | 116 |
| | VH FR1 | QVQLKESGPGLVAPSQSLSITCTVS | 52 |
| | VH FR2 | WVRQPPGKGLEWLG | 117 |
| | VH FR3 | YHSGLISRLSISKDNSKSQVFLKLNSLQSDDTATYYCAR | 118 |
| | VH FR4 | WGQGTLVTVSA | 119 |
| | VH | QVQLKESGPGLVAPSQSLSITCTVSGFSLTSYGVSWVROPP | 56 |
| | | GKGLEWLGVIWGDGSTNYHSGLISRLSISKDNSKSQVFLKL | |
| | | NSLOSDDTATYYCARVVYWGQGTLVTVSA | |
| | VL CDR1 | RSSKSLLHSNGKTYLY | 120 |
| | VL CDR2 | RMSNLAS | 121 |
| | VL CDR3 | MQHLEYPYT | 122 |
| | VL FR1 | DIVMTQAAP SVPVTPGESISISC | 123 |
| | VL FR2 | WFLQRPGQSPQLLL | 124 |
| | VL FR3 | GVPDRFSGSGSGTAFTLRISRVEAEDVGVYYC | 125 |
| | VL FR4 | FGGGTKLEIK | 47 |
| | VL | DIVMTQAAPSVPVTPGESISISCRSSKSLLHSNGKTYLY | 111 |
| | | WFLQRPGQSPQLLLYRMSNLASGVPDRFSGSGSGTAFTL | |
| | | RISRVEAEDVGVYYCMQHLEYPYTFGGGTKLEIK | |
| HFB2- | VH CDR1 | GFSLTSYGVS | 114 |
| 4hz37hG1 | VH CDR2 | VIWGDGSTN | 115 |
| | VH CDR3 | VVY | 116 |
| | VH FR1 | QVQLQESGPGLVKPSETLSLTCTVS | 93 |
| | VH FR2 | WVRQPPGKGLEWIG | 126 |
| | VH FR3 | YNPSLKSRVTISKDTSKNQVSLKLSSVTAADTAVYYCAR | 127 |
| | VH FR4 | WGQGTLVTVSS | 87 |
| | VH (VH3) | QVQLQESGPGLVKPSETLSLTCTVSGFSLTSYGVSWVRQ | 96 |
| | | PPGKGLEWIGVIWGDGSTNYNPSLKSRVTISKDTSKNQV | |
| | | SLKLSSVTAADTAVYYCARVVYWGQGTLVTVSS | |
| | VL CDR1 | RSSKSLLHSNGKTYLY | 120 |
| | VL CDR2 | RMSNLAS | 121 |
| | VL CDR3 | MQHLEYPYT | 122 |
| | VL FR1 | EIVMTQSPATLSVSPGERATLSC | 128 |
| | VL FR2 | WFQQKPGQAPRLLIY | 129 |
| | VL FR3 | GIPARFSGSGSGTEFTLTISSVQSEDVAVYYC | 130 |
| | VL FR4 | FGQGTKLEIK | 131 |
| | VL (VL6) | EIVMTQSPATLSVSPGERATLSCRSSKSLLHSNGKTYLY | 112 |
| | | WFQQKPGQAPRLLIYRMSNLASGIPARFSGSGSGTEFTL | |
| | | TISSVQSEDVAVYYCMQHLEYPYTFGQGTKLEIK | |
| HFB2- | VH CDR1 | GFSLTSYGVS | 114 |
| 4hz42hG1 | VH CDR2 | VIWGDGSTN | 115 |
| | VH CDR3 | VVY | 116 |
| | VH FR1 | QVQLVOSGAEVKKPGASVKVSCKAS | 132 |
| | VH FR2 | WVRQAPGQGLEWMG | 133 |
| | VH FR3 | YAQKLQGRVTMTKDTSTSTAYMELRSLRSDDTAVYYCAR | 134 |
| | VH FR4 | WGQGTLVTVSS | 87 |
| | VH (VH10) | QVQLVQSGAEVKKPGASVKVSCKASGFSLTSYGVSWVRQ | 113 |
| | | APGQGLEWMGVIWGDGSTNYAQKLQGRVTMTKDTSTSTA | |
| | | YMELRSLRSDDTAVYYCARVVYWGQGTLVTVSS | |
| | VL CDR1 | RSSKSLLHSNGKTYLY | 120 |
| | VL CDR2 | RMSNLAS | 121 |
| | VL CDR3 | MQHLEYPYT | 122 |
| | VL FR1 | EIVMTQSPATLSVSPGERATLSC | 128 |
| | VL FR2 | WFQQKPGQAPRLLIY | 129 |
| | VL FR3 | GIPARFSGSGSGTEFTLTISSVQSEDVAVYYC | 130 |
| | VL FR4 | FGQGTKLEIK | 131 |
| | VL (VL6) | EIVMTQSPATLSVSPGERATLSCRSSKSLLHSNGKTYLY | 112 |
| | | WFQQKPGQAPRLLIYRMSNLASGIPARFSGSGSGTEFTL | |
| | | TISSVQSEDVAVYYCMQHLEYPYTFGQGTKLEIK | |

In some embodiments, the monoclonal antibody or antigen-binding fragment thereof of the invention has a heavy chain variable region (VH) comprising one, two, three, or all (i.e., four) of a heavy chain framework region 1 (VH FR1), a heavy chain framework region 2 (VH FR2), a heavy chain framework region 3 (VH FR3), and/or a heavy chain framework region 4 (VH FR4) of the corresponding heavy chain framework regions of any one of the antibodies listed in Table B, or a VH FR1, VH FR2, VH FR3 and/or VH FR4 sequences comprising sequences substantially identical (e.g., having at least about 80%, 85%, 90%, 92%, 95%, 97%, 98%, or 99% sequence identity) to the corresponding VH FR amino acid sequences of any one of the antibodies as described in Tables B and D.

In some embodiments, the monoclonal antibodies of the invention or antigen-binding fragments thereof comprises the VH FR1, VH FR2, VHFR3 and VH FR4 sequences comprising amino acid sequences substantially identical (e.g., having at least about 80%, 85%, 90%, 92%, 95%, 97%, 98%, or 99% sequence identity) or identical to the amino acid sequences of SEQ ID NOs: 84, 85, 86 and 87, respectively.

In some embodiments, the monoclonal antibodies of the invention or antigen-binding fragments thereof comprises the VH FR1, VH FR2, VHFR3 and VH FR4 sequences comprising amino acid sequences substantially identical (e.g., having at least about 80%, 85%, 90%, 92%, 95%, 97%, 98%, or 99% sequence identity) or identical to the amino acid sequences of SEQ ID NOs: 89, 90, 91 and 87, respectively.

In some embodiments, the monoclonal antibodies of the invention or antigen-binding fragments thereof comprises the VH FR1, VH FR2, VHFR3 and VH FR4 sequences comprising amino acid sequences substantially identical (e.g., having at least about 80%, 85%, 90%, 92%, 95%, 97%, 98%, or 99% sequence identity) or identical to the amino acid sequences of SEQ ID NOs: 93, 94, 95 and 87, respectively.

In some embodiments, the monoclonal antibodies of the invention or antigen-binding fragments thereof comprises the VH FR1, VH FR2, VHFR3 and VH FR4 sequences comprising amino acid sequences substantially identical (e.g., having at least about 80%, 85%, 90%, 92%, 95%, 97%, 98%, or 99% sequence identity) or identical to the amino acid sequences of SEQ ID NOs: 138, 94, 139 and 87, respectively.

In some embodiments, the monoclonal antibodies of the invention or antigen-binding fragments thereof comprises the VH FR1, VH FR2, VHFR3 and VH FR4 sequences comprising amino acid sequences substantially identical (e.g., having at least about 80%, 85%, 90%, 92%, 95%, 97%, 98%, or 99% sequence identity) or identical to the amino acid sequences of SEQ ID NOs: 141, 142, 143 and 87, respectively.

In some embodiments, the monoclonal antibodies of the invention or antigen-binding fragments thereof comprises the VH FR1, VH FR2, VHFR3 and VH FR4 sequences comprising amino acid sequences substantially identical (e.g., having at least about 80%, 85%, 90%, 92%, 95%, 97%, 98%, or 99% sequence identity) or identical to that of SEQ ID NOs: 132, 85, 133 and 87, respectively;

In some embodiments, the monoclonal antibodies of the invention or antigen-binding fragments thereof comprises the VH FR1, VH FR2, VHFR3 and VH FR4 sequences comprising amino acid sequences substantially identical (e.g., having at least about 80%, 85%, 90%, 92%, 95%, 97%, 98%, or 99% sequence identity) or identical to that of SEQ ID NOs: 93, 126, 127 and 87, respectively;

In some embodiments, the monoclonal antibodies of the invention or antigen-binding fragments thereof comprises the VH FR1, VH FR2, VHFR3 and VH FR4 sequences comprising amino acid sequences substantially identical (e.g., having at least about 80%, 85%, 90%, 92%, 95%, 97%, 98%, or 99% sequence identity) or identical to that of SEQ ID NOs: 132, 133, 134 and 87, respectively

In some embodiments, the monoclonal antibody or antigen-binding fragment thereof of the invention has a light chain variable region (VL) comprising one, two, three, or all (i.e., four) of a heavy chain framework region 1 (VL FR1), a heavy chain framework region 2 (VL FR2), a heavy chain framework region 3 (VL FR3), and/or a heavy chain framework region 4 (VL FR4) of the corresponding heavy chain framework regions of any one of the antibodies listed in Table C, or a VL FR1, VL FR2, VL FR3 and/or VL FR4 comprising sequences substantially identical (e.g., having at least about 80%, 85%, 90%, 92%, 95%, 97%, 98%, or 99% sequence identity) to the corresponding VL FR amino acid sequences of any one of the antibodies as described in Tables C and D.

In some embodiments, the monoclonal antibodies of the invention or antigen-binding fragments thereof comprises the VL FR1, VL FR2, VLFR3 and VL FR4 sequences comprising amino acid sequences substantially identical (e.g., having at least about 80%, 85%, 90%, 92%, 95%, 97%, 98%, or 99% sequence identity) or identical to the amino acid sequences of SEQ ID NOs: 97, 98, 99 and 100, respectively.

In some embodiments, the monoclonal antibodies of the invention or antigen-binding fragments thereof comprises the VL FR1, VL FR2, VLFR3 and VL FR4 sequences comprising amino acid sequences substantially identical (e.g., having at least about 80%, 85%, 90%, 92%, 95%, 97%, 98%, or 99% sequence identity) or identical to the amino acid sequences of SEQ ID NOs: 97, 102, 99 and 100, respectively.

In some embodiments, the monoclonal antibodies of the invention or antigen-binding fragments thereof comprises the VL FR1, VL FR2, VLFR3 and VL FR4 sequences comprising amino acid sequences substantially identical (e.g., having at least about 80%, 85%, 90%, 92%, 95%, 97%, 98%, or 99% sequence identity) or identical to the amino acid sequences of SEQ ID NOs: 103, 104, 105 and 100, respectively.

In some embodiments, the monoclonal antibodies of the invention or antigen-binding fragments thereof comprises the VL FR1, VL FR2, VLFR3 and VL FR4 sequences comprising amino acid sequences substantially identical (e.g., having at least about 80%, 85%, 90%, 92%, 95%, 97%, 98%, or 99% sequence identity) or identical to the amino acid sequences of SEQ ID NOs: 103, 107, 108 and 100, respectively.

In some embodiments, the monoclonal antibodies of the invention or antigen-binding fragments thereof comprises the VL FR1, VL FR2, VLFR3 and VL FR4 sequences comprising amino acid sequences substantially identical (e.g., having at least about 80%, 85%, 90%, 92%, 95%, 97%, 98%, or 99% sequence identity) or identical to that of SEQ ID NOs: 134, 135, 136 and 131, respectively;

In some embodiments, the monoclonal antibodies of the invention or antigen-binding fragments thereof comprises the VL FR1, VL FR2, VLFR3 and VL FR4 sequences comprising amino acid sequences substantially identical (e.g., having at least about 80%, 85%, 90%, 92%, 95%, 97%, 98%, or 99% sequence identity) or identical to that of SEQ ID NOs: 128, 129, 130 and 131, respectively.

In some embodiments, the monoclonal antibodies of the invention or antigen-binding fragments thereof comprises the VL FR1, VL FR2, VLFR3 and VL FR4 sequences comprising amino acid sequences substantially identical (e.g., having at least about 80%, 85%, 90%, 92%, 95%, 97%, 98%, or 99% sequence identity) or identical to the amino acid sequences of SEQ ID NOs: 145, 146, 147 and 131, respectively.

In some embodiments, the monoclonal antibodies of the invention or antigen-binding fragments thereof comprises the VL FR1, VL FR2, VLFR3 and VL FR4 sequences comprising amino acid sequences substantially identical (e.g., having at least about 80%, 85%, 90%, 92%, 95%, 97%, 98%, or 99% sequence identity) or identical to the amino acid sequences of SEQ ID NOs: 97, 98, 152 and 100, respectively.

In some embodiments, the monoclonal antibodies of the invention or antigen-binding fragments thereof comprises the VL FR1, VL FR2, VLFR3 and VL FR4 sequences comprising amino acid sequences substantially identical (e.g., having at least about 80%, 85%, 90%, 92%, 95%, 97%, 98%, or 99% sequence identity) or identical to the amino acid sequences of SEQ ID NOs: 154, 102, 99 and 47, respectively.

TABLE E
Selected humanized antibodies derived from HFB2-4

| Antibody | VH | VL | VH sequence | VL sequence |
|---|---|---|---|---|
| HFB2- | VH1 | VL2 | QVQLVOSGAEVKKPGSSVKVSC | DIVMTQSPLSLPVTPGEPASIS |
| 4hz2hG1 | | | KASGFSLTSYGVSWVRQAPGQG | CRSSKSLLHSNGKTYLYWFLQK |
| | | | LEWMGVIWGDGSTNYAQKFQGR | PGQSPQLLLYRMSNLASGVPDR |
| | | | VTITKDESTSTAYMELSSLRSE | FSGSGSGTDFTLKISRVEAEDV |
| | | | DTAVYYCARVVYWGQGTLVTVS | GVYYCMQHLEYPYTFGGGTKVE |
| | | | S (SEQ ID NO: 88) | IK (SEQ ID NO: 137) |
| HFB2- | VH2 | VL1 | EVQLVESGGGLVQPGGSLRLSC | DIVMTQSPLSLPVTPGEPASIS |
| 4hz5hG1 | | | AASGFSLTSYGVSWVRQAPGKG | CRSSKSLLHSNGKTYLYWFLQK |
| | | | LEWVGVIWGDGSTNYAASVKGR | PGQSPQLLIYRMSNLASGVPDR |
| | | | FTISRDDSKNSVYLQMNSLKTE | FSGSGSGTDFTLKISRVEAEDV |
| | | | DTAVYYCARVVYWGQGTLVTVS | GVYYCMQHLEYPYTFGGGTKVE |
| | | | S (SEQ ID NO: 92) | IK (SEQ ID NO: 101) |
| HFB2- | VH2 | VL2 | EVQLVESGGGLVQPGGSLRLSC | DIVMTQSPLSLPVTPGEPASIS |
| 4hz6hG1 | | | AASGFSLTSYGVSWVRQAPGKG | CRSSKSLLHSNGKTYLYWFLOK |
| | | | LEWVGVIWGDGSTNYAASVKGR | PGQSPQLLLYRMSNLASGVPDR |
| | | | FTISRDDSKNSVYLOMNSLKTE | FSGSGSGTDFTLKISRVEAEDV |
| | | | DTAVYYCARVVYWGQGTLVTVS | GVYYCMQHLEYPYTFGGGTKVE |
| | | | S (SEQ ID NO: 92) | IK (SEQ ID NO: 137) |
| HFB2- | VH3 | VL1 | QVQLQESGPGLVKPSETLSLTC | DIVMTQSPLSLPVTPGEPASIS |
| 4hz9hG1 | | | TVSGFSLTSYGVSWVRQPPGKG | CRSSKSLLHSNGKTYLYWFLOK |
| | | | LEWIGVIWGDGSTNYNPSLKSR | PGQSPQLLIYRMSNLASGVPDR |
| | | | VTISKDTSKNQVSLKLSSVTAA | FSGSGSGTDFTLKISRVEAEDV |
| | | | DTAVYYCARVVYWGQGTLVTVS | GVYYCMQHLEYPYTFGGGTKVE |
| | | | S (SEQ ID NO: 96) | IK (SEQ ID NO: 101) |
| HFB2- | VH3 | VL2 | QVQLQESGPGLVKPSETLSLTC | DIVMTQSPLSLPVTPGEPASIS |
| 4hz10hG1 | | | TVSGFSLTSYGVSWVRQPPGKG | CRSSKSLLHSNGKTYLYWFLQK |
| | | | LEWIGVIWGDGSTNYNPSLKSR | PGQSPQLLLYRMSNLASGVPDR |
| | | | VTISKDTSKNQVSLKLSSVTAA | FSGSGSGTDFTLKISRVEAEDV |
| | | | DTAVYYCARVVYWGQGTLVTVS | GVYYCMQHLEYPYTFGGGTKVE |
| | | | S | IK (SEQ ID NO: 137) |
| HFB2- | VH3 | VL3 | QVQLQESGPGLVKPSETLSLTC | DIQMTQSPSSLSASVGDRVTIT |
| 4hz11hG1 | | | TVSGFSLTSYGVSWVRQPPGKG | CRSSKSLLHSNGKTYLYWFQQK |
| | | | LEWIGVIWGDGSTNYNPSLKSR | PGKAPKLLLYRMSNLASGVPSR |
| | | | VTISKDTSKNQVSLKLSSVTAA | FSGSGSGTDFTLTISSLQPEDV |
| | | | DTAVYYCARVVYWGQGTLVTVS | ATYYCMQHLEYPYTFGGGTKVE |
| | | | S (SEQ ID NO: 96) | IK (SEQ ID NO: 106) |
| HFB2- | VH3 | VL4 | QVQLQESGPGLVKPSETLSLTC | DIQMTQSPSSLSASVGDRVTIT |
| 4hz12hG1 | | | TVSGFSLTSYGVSWVRQPPGKG | CRSSKSLLHSNGKTYLYWFQQK |
| | | | LEWIGVIWGDGSTNYNPSLKSR | PGKAPKLLIYRMSNLASGVPSR |
| | | | VTISKDTSKNQVSLKLSSVTAA | FSGSGSGTDFTLTISSLQPEDF |
| | | | DTAVYYCARVVYWGQGTLVTVS | ATYYCMQHLEYPYTFGGGTKVE |
| | | | S (SEQ ID NO: 96) | IK (SEQ ID NO: 109) |
| HFB2- | VH3 | VL1b | QVQLQESGPGLVKPSETLSLTC | DIVMTQSPLSLPVTPGEPASIS |
| 4hz14hG1 | | | TVSGFSLTSYGVSWVRQPPGKG | CRSSQSLLHSNGYNYLYWFLQK |
| | | | LEWIGVIWGDGSTNYNPSLKSR | PGQSPOLLIYRGSNRASGVPDR |
| | | | VTISKDTSKNQVSLKLSSVTAA | FSGSGSGTDFTLKISRVEAEDV |
| | | | DTAVYYCARVVYWGQGTLVTVS | GVYYCMQHLQYPYTFGGGTKVE |
| | | | S (SEQ ID NO: 96) | IK (SEQ ID NO: 153) |
| HFB2- | VH3 | VL1c | QVQLQESGPGLVKPSETLSLTC | DIVMTQSPLSLPVTPGEPISLS |
| 4hz15hG1 | | | TVSGFSLTSYGVSWVRQPPGKG | CRSSKSLLHSNGKTYLYWFLQK |
| | | | LEWIGVIWGDGSTNYNPSLKSR | PGQSPOLLLYRMSNLASGVPDR |
| | | | VTISKDTSKNQVSLKLSSVTAA | FSGSGSGTDFTLKISRVEAEDV |
| | | | DTAVYYCARVVYWGQGTLVTVS | GVYYCMQHLEYPYTFGGGTKLE |
| | | | S (SEQ ID NO: 96) | IK (SEQ ID NO: 155) |
| HFB2- | VH3 | VL6 | QVQLQESGPGLVKPSETLSLTC | EIVMTQSPATLSVSPGERATLS |
| 4hz37hG1 | | | TVSGFSLTSYGVSWVRQPPGKG | CRSSKSLLHSNGKTYLYWFQQK |
| | | | LEWIGVIWGDGSTNYNPSLKSR | PGQAPRLLIYRMSNLASGIPAR |
| | | | VTISKDTSKNQVSLKLSSVTAA | FSGSGSGTEFTLTISSVQSEDV |
| | | | DTAVYYCARVVYWGQGTLVTVS | AVYYCMQHLEYPYTFGQGTKLE |
| | | | S (SEQ ID NO: 96) | IK (SEQ ID NO: 112) |
| HFB2- | VH3 | VL7 | QVQLQESGPGLVKPSETLSLTC | DIVMTQSPDSLAVSLGERATIN |
| 4hz38hG1 | | | TVSGFSLTSYGVSWVRQPPGKG | CRSSKSLLHSNGKTYLYWFQQK |
| | | | LEWIGVIWGDGSTNYNPSLKSR | PGQSPKLLLYRMSNLASGVPDR |
| | | | VTISKDTSKNQVSLKLSSVTAA | FSGSGSGTDFTLTISSLQAEDV |
| | | | DTAVYYCARVVYWGQGTLVTVS | AVYYCMQHLEYPYTFGQGTKLE |
| | | | S (SEQ ID NO: 96) | IK (SEQ ID NO: 148) |
| HFB2- | VH6 | VL6 | QVQLQESGPGLVKPSGTLSLTC | EIVMTQSPATLSVSPGERATLS |
| 4hz39hG1 | | | AVSGFSLTSYGVSWVRQPPGKG | CRSSKSLLHSNGKTYLYWFQQK |
| | | | LEWIGVIWGDGSTNYNPSLKSR | PGQAPRLLIYRMSNLASGIPAR |
| | | | LTISKDKSKNQVSLKLSSVTAA | FSGSGSGTEFTLTISSVQSEDV |
| | | | DTAVYYCARVVYWGQGTLVTVS | AVYYCMQHLEYPYTFGQGTKLE |
| | | | S (SEQ ID NO: 140) | IK (SEQ ID NO: 112) |
| HFB2- | VH8 | VL7 | QVQLVESGGGVVQPGRSLRLSC | DIVMTQSPDSLAVSLGERATIN |
| 4hz41hG1 | | | AVSGFSLTSYGVSWVRQAPGKG | CRSSKSLLHSNGKTYLYWFQQK |
| | | | LEWLGVIWGDGSTNYHSGLISR | PGQSPKLLLYRMSNLASGVPDR |
| | | | LTISKDNSKNTVYLOMNSLRAE | FSGSGSGTDFTLTISSLQAEDV |
| | | | DTAVYYCARVVYWGQGTLVTVS | AVYYCMQHLEYPYTFGQGTKLE |
| | | | S (SEQ ID NO: 144) | IK (SEQ ID NO: 148) |
| HFB2- | VH10 | VL6 | QVQLVQSGAEVKKPGASVKVSC | EIVMTQSPATLSVSPGERATLS |
| 4hz42hG1 | | | KASGFSLTSYGVSWVRQAPGQG | CRSSKSLLHSNGKTYLYWFQQK |
| | | | LEWMGVIWGDGSTNYAQKLQGR | PGQAPRLLIYRMSNLASGIPAR |
| | | | VTMTKDTSTSTAYMELRSLRSD | FSGSGSGTEFTLTISSVQSEDV |
| | | | DTAVYYCARVVYWGQGTLVTVS | AVYYCMQHLEYPYTFGQGTKLE |
| | | | S (SEQ ID NO: 113) | IK (SEQ ID NO: 112) |

In some embodiments, a humanized antibody also comprises a human heavy chain constant region and/or a human light chain constant region.

### 8. Human Antibodies

In some embodiments, the antibody of the invention is a human antibody. Human antibodies can be made by any suitable method. Non-limiting exemplary methods include making human antibodies in transgenic mice that comprise human immunoglobulin loci. See, e.g., Jakobovits et al., Proc. Natl. Acad. Sci. USA 90: 2551-55 (1993); Jakobovits et al, Nature 362: 255-8 (1993); onberg et al, Nature 368: 856-9 (1994); and U.S. Pat. Nos. 5,545,807; 6,713,610; 6,673,986; 6,162,963; 5,545,807; 6,300,129; 6,255,458; 5,877,397; 5,874,299; and 5,545,806.

Non-limiting exemplary methods also include making human antibodies using phage display libraries. See, e.g., Hoogenboom et al., J. Mol. Biol. 227: 381-8 (1992); Marks et al, J. Mol. Biol. 222: 581-97 (1991); and PCT Publication No. WO 99/10494.

Antibody Constant Regions

In some embodiments, a humanized, chimeric, or human antibody described herein comprises one or more human constant regions. In some embodiments, the human heavy chain constant region is of an isotype selected from IgA, IgG, and IgD. In some embodiments, the human light chain constant region is of an isotype selected from K and k. In some embodiments, an antibody described herein comprises a human IgG constant region, for example, human IgG1, IgG2, IgG3, or IgG4. In some embodiments, an antibody or Fc fusion partner comprises a C237S mutation, for example, in an IgG1 constant region. In some embodiments, an antibody described herein comprises a human IgG2 heavy chain constant region. In some such embodiments, the IgG2 constant region comprises a P331S mutation, as described in U.S. Pat. No. 6,900,292. In some embodiments, an antibody described herein comprises a human IgG4 heavy chain constant region. In some such embodiments, an antibody described herein comprises an S241P mutation in the human IgG4 constant region. See, e.g., Angal et al. Mol. Immunol. 30(1):105-108 (1993). In some embodiments, an antibody described herein comprises a human IgG4 constant region and a human κ light chain.

The choice of heavy chain constant region can determine whether or not an antibody will have effector function in vivo. Such effector function, in some embodiments, includes antibody-dependent cell-mediated cytotoxicity (ADCC) and/or complement-dependent cytotoxicity (CDC), and can result in killing of the cell to which the antibody is bound. Typically, antibodies comprising human IgG1 or IgG3 heavy chains have effector function.

In some embodiments, effector function is not desirable. For example, in some embodiments, effector function may not be desirable in treatments of inflammatory conditions and/or autoimmune disorders. In some such embodiments, a human IgG4 or IgG2 heavy chain constant region is selected or engineered. In some embodiments, an IgG4 constant region comprises an S241P mutation.

Any of the antibodies described herein may be purified by any suitable method. Such methods include, but are not limited to, the use of affinity matrices or hydrophobic interaction chromatography. Suitable affinity ligands include the antigen and/or epitope to which the antibody binds, and ligands that bind antibody constant regions. For example, a Protein A, Protein G, Protein A/G, or an antibody affinity column may be used to bind the constant region and to purify an antibody.

In some embodiments, hydrophobic interactive chromatography (HIC), for example, a butyl or phenyl column, is also used for purifying some polypeptides. Many methods of purifying polypeptides are known in the art.

Alternatively, in some embodiments, an antibody described herein is produced in a cell-free system. Nonlimiting exemplary cell-free systems are described, e.g., in Sitaraman et al., Methods Mol. Biol. 498: 229-44 (2009); Spirin, Trends Biotechnol. 22: 538-45 (2004); Endo et al, Biotechnol. Adv. 21: 695-713 (2003).

### 9. Antibody Fucosylation and Afucosylation

One aspect of the invention provides fucosylated and afucosylated antibodies that bind CXCR5, and compositions comprising such antibodies as well as uses for such antibodies, including therapeutic and pharmaceutical uses.

Alternatively, the invention provides anti-CXCR5 antibodies that exhibit an altered effector function. In some embodiments, the altered effector function is increased ADCC. In some embodiments, the antibodies lack, or contain detectably decreased levels (e.g., <10%, <5%, <2%) of fucose (i.e., they are afucosylated).

In some embodiments, afucosylated antibody heavy chains and light chains that are capable of forming antibodies that bind CXCR5 are provided. In some embodiments, afucosylated antibodies, heavy chains, and light chains comprising one or more particular complementarity determining regions (CDRs) are provided. In some embodiments, afucosylated anti-CXCR5 antibodies have altered effector function. In some embodiments, the antibodies of the invention have enhanced ADCC activity relative to otherwise identical fucosylated anti-CXCR5 antibodies of the invention.

L-fucose (or 6-deoxy-L-galactose) is a monosaccharide that is a component of some N- and O-linked glycans and glycolipids in animals (Becker and Lowe, Glycobiology 13:41R-51R, 2003, incorporated by reference). Fucose is typically added as a terminal modification to glycans, including glycans attached to blood group antigens, selectins and antibodies. Fucose can be attached to glycans via α(1,2)-, α(1,3)-, α(1,4)- and α(1,6)-linkages by specific fucosyltransferases. α(1,2)-fucose linkages are typically associated with the H-blood group antigens. α(1,3)- and α(1,4)-fucose linkages are associated with modification of LewisX antigens. α(1,6)-fucose linkages are associated with N-linked GlcNAc molecules, such as those on antibodies.

An “afucosylated” antibody of the invention includes an antibody lacking fucose, e.g., an IgG1 or IgG3 isotype antibody that lacks fucose in its constant region glycosylation. Glycosylation of human IgG1 or IgG3 occurs at Asn297 as core fucosylated biantennary complex oligosaccharide glycosylation terminated with up to 2 Gal residues.

The major types of complex oligosaccharide structures, or “glycoforms,” found in the CH2 domain of the IgG, are described in WO 99/22764 (incorporated by reference, see page 7). Here, G0 refers to a biantennary structure wherein no terminal sialic acids (NeuAcs) or Gals are present, G1 refers to a biantennary structure having one Gal and no NeuAcs and G2 refers to a biantennary structure with two terminal Gals and no NeuAcs.

In some embodiments, an afucosylated antibody of the invention lacks fucose at Asn297. These structures are designated as G0, G1 (a 1,6 or a 1,3) or G2 glycan residues, depending on the amount of terminal Gal residues. See, e.g., Raju, BioProcess Int.1: 44-53 (2003). CHO type glycosylation of antibody Fc is described, e.g., in Routier, Glycoconjugate J. 14: 201-207 (1997).

In some embodiments, the “afucosyl” or“afucosylated,” as used interchangeably herein, antibody refers to an antibody that has been glycoengineered to lack core fucose. Antibodies with reduced fucose content in glycan moieties have increased affinity to FcγRIIIa (CD16), and as a result, possess enhanced activity-dependent cellular cytotoxicity (ADCC) activity.

Afucosyl antibodies can be produced using the POTELLIGENT® CHOK1SV cell line (Lonza Biologics), which lacks both alleles of the gene responsible for fucose addition (α1,6-fucosyltransferase). Afucosyl or reduced fucose antibodies can also be generated by modifying the oligosaccharide biosynthesis activities in various ways. For example, overexpression of N-acetylglucosamine-transferase III (GnTIII) in the Golgi apparatus of the production cell line generates bisected oligosaccharide structures associated with the Fc constant region of the antibody and suppresses fucosylation. In such expression systems, the level of GnTIII expression correlates with the generation of afucosylated IgG1 glycoforms and resulting enhanced ADCC activity.

Fucosylation can also be decreased in cell culture by use of sugar analogs, such as, but not limited to, fucose analogs as described in WO 2012/019165. Thus, afucosylated, or reduced fucose, antibodies can be produced using a wide variety of methods well-known in the art.

In some embodiments, an afucosylated antibody of the invention has enhanced affinity for Fc gamma RIIIA In some embodiments, an afucosylated antibody of the invention has enhanced affinity for Fc gamma RIIIA(V158). In some embodiments, an afucosylated antibody of the invention has enhanced affinity for Fc gamma RIIIA(F158).

“Glycoform” refers to a complex oligosaccharide structure comprising linkages of various carbohydrate units. Such structures are described in, e.g., Essentials of Glycobiology Varki et al., eds., Cold Spring Harbor Laboratory Press, Cold Spring Harbor, NY (1999), which also provides a review of standard glycobiology nomenclature. Such glycoforms include, but are not limited to, G2, G1, G0, G−1, and G−2 (see, e.g., International Patent Publication No. WO 99/22764).

“Glycosylation pattern” is defined as the pattern of carbohydrate units that are covalently attached to a protein (e.g., the glycoform) as well as to the site(s) to which the glycoform(s) are covalently attached to the peptide backbone of a protein, more specifically to an immunoglobulin protein.

In some embodiments, at least 85 percent of a batch of antibodies recombinantly expressed in non glycomodified CHO host cells are fucosylated at Asn297.

When referring to a composition comprising a plurality of antibodies, the antibodies are considered to be afucosylated if less than about 5 percent of the antibodies in the composition comprise fucose at at least one Asn297.

In certain embodiments, the level of afucosylation is about 100%, that is, no fucose is detected on either heavy chain Asn297 glycoform using standard methods for measuring fucosylation of an antibody.

Methods of measuring fucose include any methods known in the art, including the methods described herein. In some embodiments, fucose is undetectable in a composition comprising a plurality of afucosylated antibodies. In some embodiments, an afucosylated antibody has enhanced ADCC activity.

In some embodiments, antibodies are provided having a carbohydrate structure that lacks fucose attached (directly or indirectly) to an Fc region (i.e., afucosylated antibodies). For example, the amount of fucose in a composition comprising a plurality of such antibodies may be 0-30%, 0-20%, 0-15%, 1-10%, or 0-5%.

In some embodiments, a composition comprising a plurality of the antibody of the invention comprises >80%, 85%, 90%, 95%, 97%, 99%, or >99.5% afucosylated antibodies. In some embodiments, the antibodies are 100 percent afucosylated within the detection limit (i.e., fucose is undetectable/undetected at Asn297 using an art-recognized method for detecting fucose in an antibody, such as the one described herein). The amount of fucose can be determined by calculating the average amount of fucose within the sugar chain at Asn297, relative to the sum of all glycostructures attached to Asn 297 (e.g., complex, hybrid and high mannose structures).

In some embodiments, the level of fucosylation is no more than 0.5%, which is based on the limit of quantification (LOQ) for the test method. Thus, in some embodiments, the level of afucosylation is greater than or equal to 99.5%.

The N-linked oligosaccharide profile method can be used to determine the level of fucosylation, sialylation, mannosylation, and terminal galactosylation in a sample. The N-linked Oligosaccharide method can be used to evaluate N-linked glycans. Briefly, N-linked glycans are enzymatically released from the protein with peptide-N-glycosidase F. The glycans are then derivatized by a fluorescent agent and analyzed using hydrophilic interaction liquid chromatography and fluorescence detection. The chromatographic profile is then compared to that of the reference material. This and many other methods known in the art can be used to assess the degree of fucosylation of an antibody, and can be used to determine the level of fucosylation present in the antibody of the present invention.

Non-limiting exemplary methods of detecting fucose in an antibody include MALDI-TOF mass spectrometry (see, e.g., WO 2008/077546), HPLC measurement of released fluorescently labeled oligosaccharides (see, e.g., Schneider et al., N-Glycan analysis of monoclonal antibodies and other glycoproteins using UHPLC with fluorescence detection, Agilent Technologies, Inc. (2012); Lines, J. Pharm. Biomed. Analysis 14: 601-608 (1996); Takahasi, J. Chrom. 720: 217-225 (1996)), capillary electrophoresis measurement of released fluorescently labeled oligosaccharides (see, e.g., Ma et al., Anal. Chem., 71: 5185-5192 (1999)), and HPLC with pulsed amperometric detection to measure monosaccharide composition (see, e.g., Hardy et al, Analytical Biochem. 170: 54-62 (1988)).

Asn297 refers to the asparagine residue located at about position 297 in the Fc region (EU numbering of Fc region residues); however, Asn297 may also be located about plus or minus 3 amino acids upstream or downstream of position 297, i.e., between positions 294 and 300, due to minor sequence variations in antibodies. In a CXCR5 antibody described herein, Asn297 may be found in the sequence motif QYNST.

Fucosylation variants may have improved ADCC function. See, e.g., US 2003/0157108 and US 2004/0093621. Examples of publications related to “afucosylated” or “fucose-deficient” antibodies include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al., J. Mol. Biol. 336: 1239-1249 (2004); Yamane-Ohnuki et al., Biotech. Bioeng. 87: 614 (2004) (all incorporated herein by reference).

Examples of cell lines capable of producing afucosylated antibodies include Lec 13 CHO cells deficient in protein fucosylation (Ripka et al., Arch. Biochem. Biophys. 249: 533-545 (1986)); US 2003/0157108 A1; and WO 2004/056312 (see Example 11), and knockout cell lines, such as cell lines lacking a functional alpha-1,6-fucosyltransferase gene, FUT8, e.g., knockout CHO cells (see, e.g., Yamane-Ohnuki et al., Biotech. Bioeng. 87: 614 (2004); Kanda et al., Biotechnol. Bioeng, 94(4):680-688 (2006); and WO2003/085107).

In certain embodiments, the antibodies of the invention have bisected oligosaccharides, e.g., in which a biantennary oligosaccharide attached to the Fc region of the antibody is bisected by GlcNAc. Such antibodies may have reduced fucosylation and/or improved ADCC function. Examples of such antibodies are described, e.g., in WO 2003/011878; U.S. Pat. No. 6,602,684; and US 2005/0123546.

In certain embodiments, the antibodies of the invention have at least one galactose residue in the oligosaccharide attached to the Fc region. Such antibodies may have improved CDC function. Such antibodies are described, e.g., in WO 1997/30087; WO 1998/58964; and WO 1999/22764 (all incorporated herein by reference).

In some embodiments, an afucosylated antibody of the invention mediates ADCC in the presence of human effector cells more effectively than a control/parent antibody that comprises fucose. Generally, ADCC activity may be determined using the in vitro ADCC assay as herein disclosed, but other assays or methods for determining ADCC activity, e.g. in an animal model etc., are contemplated.

In some embodiments, afucosylated anti-CXCR5 antibodies have enhanced ADCC activity in vitro and/or in vivo. In some embodiments, afucosylated anti-CXCR5 antibodies have enhanced ADCC activity in vitro. In some embodiments, ADCC activity in vitro is determined by a method described herein. Briefly, serial dilutions of anti-CXCR5 antibodies or an isotype control are incubated with peripheral blood mononuclear cells (PBMCs) from healthy human donors or cynomolgus monkeys. In this assay, the PBMCs are the source of the natural killer (NK) effector cells and the target CXCR5⁺ B and Tfh-like cells. Flow cytometry is used to quantify the number of B and Tfh-like cells remaining after approximately 20 hr. The cytotoxicity titration curves were generated by plotting the percentage of cytotoxicity of the antigen binding population against the log of PF-06835375 antibody concentration. EC50 values were determined using GraphPad Prism (version 6.0, GraphPad Software, Inc, San Diego, CA) nonlinear-regression curve fits and a sigmoidal log of agonist dose-response model, according to the following equation:

Log(agonist) vs. response−variable slope(four parameters)

Y=Bottom+(Top−Bottom/(1+10\hat{ }((Log EC₅₀−X)*HillSlope))

where Y is the percentage of cytotoxicity, X is antibody concentration, Top is the maximum Y-value corresponding to the upper plateau of the sigmoidal curve, Bottom is the minimum Y-value corresponding to the lower plateau of the sigmoidal curve (constrained to 0), and Log EC50 is the log of the concentration of antibody at the inflection point of the curve.

The EC50 values were summarized across experiments using average and standard deviations (STDEV).

In some embodiments, the ability of the humanized mAbs to induce ADCC of bona fide Tfh cells from human tonsil was assessed similarly using CD4+ T cells isolated from tonsillar mononuclear cells with the addition of NK cells isolated from PBMCs.

In some embodiments, Ba/F3 cells that express CXCR5 are used as target cells. In some embodiments, cytotoxicity is determined by quantifying LDH release using CytoTox Non-Radioactive Cytotoxicity Assay (Promega, Madison, WI).

In some embodiments, maximal lysis is determined using 5% Triton X-100 and spontaneous release is determined in the absence of antibody. In some embodiments, the percentage of specific lysis may be determined using the formula: (experimental−spontaneous release)/(maximal−spontaneous release)×100=percent specific lysis.

In some embodiments, an afucosylated anti-CXCR5 antibody having enhanced ADCC activity results in specific lysis that is at least 10, at least 15, at least 20, at least 25, at least 30, at least 35, at least 40, at least 45, at least 50, at least 60, at least 65, at least 70, or at least 75 percentage points greater than specific lysis with the same amount of a fucosylated antibody, at at least one concentration of antibody tested.

In some embodiments, an afucosylated anti-CXCR5 antibody having enhanced ADCC activity results in specific lysis that is at least 10, at least 15, at least 20, at least 25, at least 30, at least 35, at least 40, at least 45, at least 50, at least 60, at least 65, at least 70, or at least 75 percentage points greater than specific lysis with a fucosylated antibody, where each antibody is at a concentration of between 0.01 and 1 microg/ml and the target cells are B a/F3 cells expressing CXCR5.

In some embodiments, the antibodies are tested at concentrations ranging from 0.000005 microg/ml to 5 microg/ml.

In some embodiments, afucosylated anti-CXCR5 antibodies have enhanced affinity for Fc gamma RIIIA In some embodiments, afucosylated anti-CXCR5 antibodies have enhanced affinity for Fc gamma RIIIA(V158). In some embodiments, afucosylated anti-CXCR5 antibodies have enhanced affinity for Fc gamma RIIIA(F158). In some embodiments, antibody affinity for Fc gamma RIIIA is determined using surface plasmon resonance and/or as follows, which is described with reference to Fc gamma RIIIA(V158), but which is also suitable for determining affinity for Fc gamma RIIIA(F158).

Briefly, in some embodiments, fucosylated or afucosylated anti-CXCR5 antibody is captured on a protein A-coated dextran chip. Fc gamma RIIIA (V158) (available from, e.g., R and D Systems) is injected at various concentrations. The association constant, dissociation constant, and affinity of Fc gamma RIIIA (V158) for fucosylated and afucosylated anti-CXCR5 antibody may be determined, e.g., using software provided with the surface plasmon resonance system (for example, Biacore T200 Evaluation Software 1:1 binding model).

In some embodiments, an afucosylated anti-CXCR5 antibody can have enhanced affinity for Fc gamma RIIIA (such as Fc gamma RIIIA(V158) or Fc gamma RIIIA(F158)) and can bind to Fc gamma RIIIA with at least 2-fold, at least 3-fold, at least 4-fold, at least 5-fold, at least 7-fold, at least 10-fold, at least 12-fold, at least 15-fold, at least 17-fold, 20-fold, 30-fold, 50-fold, 100-fold, 500-fold, or at least 1000-fold greater affinity than a fucosylated anti-CXCR5 antibody.

### 10. Nucleic Acid Molecules Encoding Antibodies of the Invention

The invention also provides nucleic acid molecules comprising polynucleotides that encode one or more chains of an antibody described herein. In some embodiments, a nucleic acid molecule comprises a polynucleotide that encodes a heavy chain or a light chain of an antibody described herein. In some embodiments, a nucleic acid molecule comprises both a polynucleotide that encodes a heavy chain and a polynucleotide that encodes a light chain, of an antibody described herein. In some embodiments, a first nucleic acid molecule comprises a first polynucleotide that encodes a heavy chain and a second nucleic acid molecule comprises a second polynucleotide that encodes a light chain.

In some such embodiments, the heavy chain and the light chain are expressed from one nucleic acid molecule, or from two separate nucleic acid molecules, as two separate polypeptides. In some embodiments, such as when an antibody is an scFv, a single polynucleotide encodes a single polypeptide comprising both a heavy chain and a light chain linked together.

In some embodiments, a polynucleotide encoding a heavy chain or light chain of an antibody described herein comprises a nucleotide sequence that encodes a leader sequence, which, when translated, is located at the N-terminus of the heavy chain or light chain. As discussed above, the leader sequence may be the native heavy or light chain leader sequence, or may be another heterologous leader sequence.

Nucleic acid molecules may be constructed using recombinant DNA techniques conventional in the art. In some embodiments, a nucleic acid molecule is an expression vector that is suitable for expression in a selected host cell, such as a mammalian cell.

### 11. Vectors

Vectors comprising polynucleotides that encode heavy chains and/or light chains of the antibodies described herein are provided. Such vectors include, but are not limited to, DNA vectors, phage vectors, viral vectors, retroviral vectors, etc. In some embodiments, a vector comprises a first polynucleotide sequence encoding a heavy chain and a second polynucleotide sequence encoding a light chain. In some embodiments, the heavy chain and light chain are expressed from the vector as two separate polypeptides. In some embodiments, the heavy chain and light chain are expressed as part of a single polypeptide, such as, for example, when the antibody is an scFv.

In some embodiments, a first vector comprises a polynucleotide that encodes a heavy chain and a second vector comprises a polynucleotide that encodes a light chain. In some embodiments, the first vector and second vector are transfected into host cells in similar amounts (such as similar molar amounts or similar mass amounts). In some embodiments, a mole- or mass-ratio of between 5:1 and 1:5 of the first vector and the second vector is transfected into host cells. In some embodiments, a mass ratio of between 1:1 and 1:5 for the vector encoding the heavy chain and the vector encoding the light chain is used. In some embodiments, a mass ratio of 1:2 for the vector encoding the heavy chain and the vector encoding the light chain is used.

In some embodiments, a vector is selected that is optimized for expression of polypeptides in CHO or CHO-derived cells, or in NS0 cells. Exemplary such vectors are described, e.g., in Running Deer et al., Biotechnol. Prog. 20:880-889 (2004). In some embodiments, a vector is chosen for in vivo expression of the subject antibodies in animals, including humans. In some such embodiments, expression of the polypeptide or polypeptides is under the control of a promoter or promoters that function in a tissue-specific manner. For example, liver-specific promoters are described, e.g., in PCT Publication No. WO 2006/076288.

### 12. Host Cells

Another aspect of the invention provides a host cell comprising the nucleic acid molecule encoding any one of the anti-CXCR5 antibodies of the invention, and/or and vector comprising said nucleic acid molecule.

In various embodiments, heavy chains and/or light chains of the antibodies described herein may be expressed in prokaryotic cells, such as bacterial cells; or in eukaryotic cells, such as fungal cells (such as yeast), plant cells, insect cells, and mammalian cells. Such expression may be carried out, for example, according to procedures known in the art.

Exemplary eukaryotic cells that may be used to express polypeptides include, but are not limited to, COS cells, including COS 7 cells; 293 cells, including 293-6E cells; CHO cells, including CHO-S and DG44 cells; PER.C6® cells (Crucell); and NS0 cells.

In certain embodiments, the cell is a mammalian cell. In certain embodiments, the host cell is a CHO cell, a COS cell, a HEK-293 cell, an NS0 cell, a PER.C6® cell, or an Sp2.0 cell. In certain embodiments, the host cell lacks a functional alpha-1,6-fucosyltransferase (FUT8). In certain embodiments, the host cell does not express a functional alpha-1,6-fucosyltransferase enzyme. In certain embodiments, the cell lacks a FUT8 gene encoding a functional enzyme. In certain embodiments, the host cell lacks a gene encoding a functional FUT8 gene. In certain embodiments, the host cell is a Potelligent® CHOK1SV cell, or a Lec13 CHO cell. In certain embodiments, the host cell is Potelligent® CHOK1SV cell.

In some embodiments, heavy chains and/or light chains of the antibodies described herein may be expressed in yeast. See, e.g., U.S. Publication No. US 2006/0270045 A1. In some embodiments, a particular eukaryotic host cell is selected based on its ability to make desired post-translational modifications to the heavy chains and/or light chains of CXCR5 antibody. For example, in some embodiments, CHO cells produce polypeptides that have a higher level of sialylation than the same polypeptide produced in 293 cells.

Introduction of one or more nucleic acids into a desired host cell may be accomplished by any method, including but not limited to, calcium phosphate transfection, DEAE-dextran mediated transfection, cationic lipid-mediated transfection, electroporation, transduction, infection, etc., Nonlimiting exemplary methods are described, e.g., in Sambrook et al., Molecular Cloning, A Laboratory Manual, 3rd ed. Cold Spring Harbor Laboratory Press (2001). Nucleic acids may be transiently or stably transfected in the desired host cells, according to any suitable method.

In some embodiments, one or more polypeptides may be produced in vivo in an animal that has been engineered or transfected with one or more nucleic acid molecules encoding the polypeptides, according to any suitable method.

Thus another related aspect of the invention provides a method of making an antibody or antigen-binding fragment thereof, comprising culturing the host cell of the invention described herein (e.g., those comprising the polynucleotide coding sequence for any one of the antibody or functional antigen-binding fragment thereof of the invention), under a condition wherein said antibody or antigen-binding fragment is expressed by said host cell.

In certain embodiments, the method further comprises isolating said antibody or antigen-binding fragment thereof.

In a related aspect, the invention provides a method of making an afucosylated anti-CXCR5 antibody, or an antigen-binding fragment thereof, said method comprising culturing a host cell comprising the nucleic acid molecule of the invention, or the vector of the invention, wherein the host cell lacks a functional FUT8.

In certain embodiments, the host cell is a Potelligent® CHOK1SV cell.

Another related aspect of the invention provides an isolated antibody, or antigen-binding fragment thereof, produced using the method of the invention.

In certain embodiments, the isolated antibody, or antigen-binding fragment thereof of the invention is afucosylated.

In certain embodiments, the afucosylated antibody, or antigen-binding fragment thereof, exhibits enhanced antibody-dependent cellular cytotoxicity (ADCC) compared with an otherwise identical antibody, or antigen-binding fragment thereof, which is fucosylated.

In certain embodiments, the afucosylated antibody, or antigen-binding fragment thereof, exhibits about 2-fold, about 5-fold, about 7-fold, about 10-fold, about 20-fold, about 30-fold, about 40-fold, about 50-fold, about 60-fold, about 70-fold, about 80-fold, about 90-fold, about 100-fold, about 110-fold, about 120-fold, about 130-fold, about 140-fold, and about 143-fold greater ADCC compared with an otherwise identical antibody, or antigen-binding fragment thereof, which is fucosylated.

Another related aspect of the invention provides a pharmaceutical composition comprising an antibody or antigen-binding fragment thereof of the invention, and a pharmaceutically acceptable carrier or excipient. In certain embodiments, the antibody, or antigen-binding fragment thereof, is afucosylated.

### EXAMPLES

### Example 1 Identification and Preliminary Characterization of Chimeric Anti-CXCR5 Monoclonal Antibodies

Three immunization campaigns were conducted with different designs, which produced a total of 185 candidate murine monoclonal antibodies, 54 of which were confirmed binders for CXCR5. Six of the confirmed binders had high binding affinity, characterized as MFI>50% of a benchmark antibody signal, and >80% of positive cells.

Chimeric monoclonal antibodies were then generated based on the variable (VH and VL) regions of the murine antibodies, and the hIgG1 scaffold/constant region of the human IgG1. The specific antibodies were characterized as % of positive cells >1 Log as compared to the % obtained with isotype control.

### Selection of Candidate Chimeric Antibodies Using In Vitro Assays

A series of in vitro characterization of the chimeric antibodies were then conducted to prioritize these antibodies. In particular, characterization of the six strong binders were based on different critical in vitro assays as binding on the cells expressing the target of interest, specificity assay, and cross-reactivity assay by flow cytometry. Several second order assays were established to rank the strong binders based on their functionality effect on B cell migration, their enzymatic intracellular agonistic activity, and receptor internalization. Most importantly, the strong candidates were then characterized in antibody dependent cell cytotoxicity (ADCC) reporter assay, as ADCC is the desired mode of action of the strong candidate antibodies. Read out of the ADCC reporter assay is luminescence signal from NFAT response element driving expression of firefly luciferase.

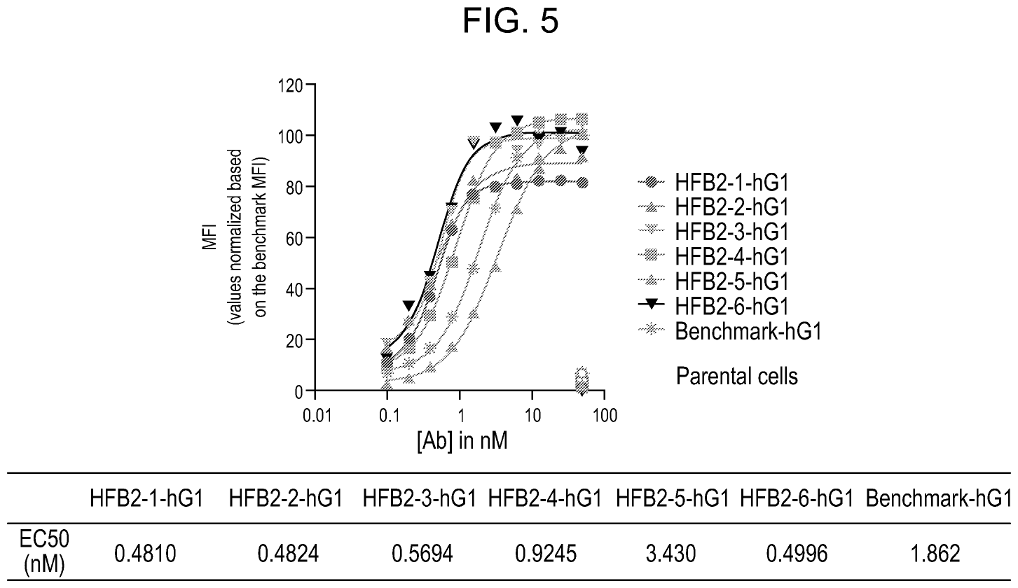

In order to test the binding capacity to CXCR5, six chimeric monoclonal antibodies were tested in binding assay using adherent stable cell line expressing the target antigen hCXCR5. The results showed that all six chimeric antibodies bound to cells expressing the target antigen with different binding properties. Among them, chimeric antibodies HFB2-3-hG1, HFB2-4-hG1, HFB2-5-hG1 and HFB2-6-hG1 showed higher binding capacity as compared to HFB2-1-hG1 and HFB2-2-hG1. All but one chimeric antibodies showed sub-nM EC50 values. See FIG. 5.

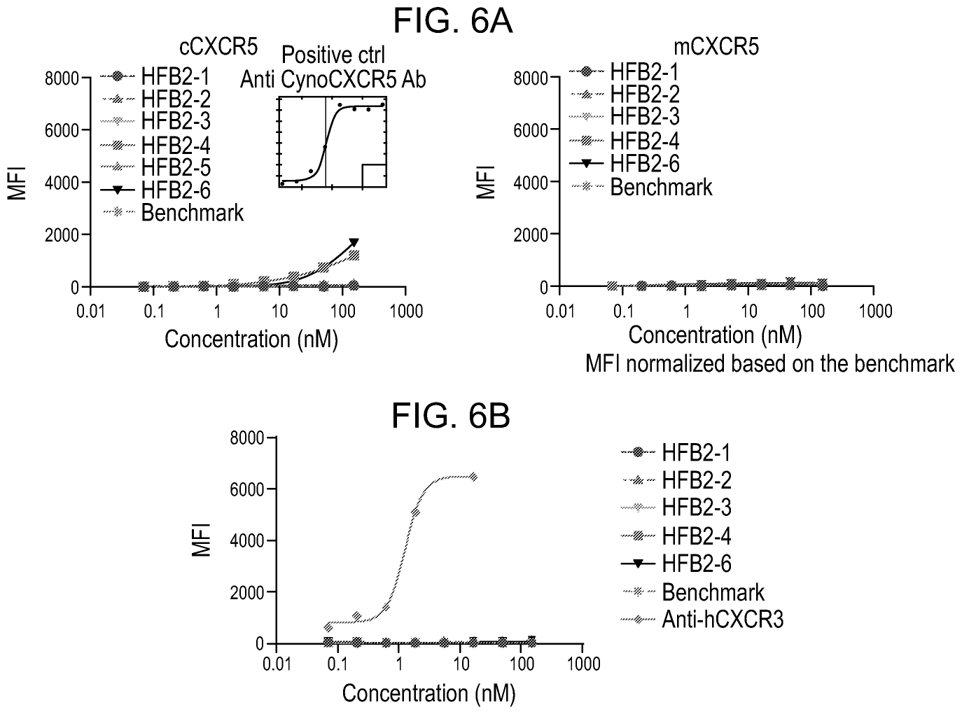

Next, cross-reactivity binding assessment showed that none of the chimeric monoclonal antibodies cross reacted with the monkey or murine CXCR5 ortholog, as compared to positive control included in the assay. See FIG. 6A. Further, none of anti-hCXCR5 chimeric antibodies binds to the hCXCR5 ortholog hCXCR3, thus confirming their specificity. See FIG. 6B.

As the target antigen CXCR5 is a GPCR involved in chemotaxis, experiments were conducted to confirm the ability of the subject antibodies to inhibit in vitro B-cell migration. As one of the specific antibody ranking assays, the B cell migration assay utilized M300.19 suspension cells expressing the CXCR5 target antigen for testing all antibody candidates as well as the benchmark/control antibody. The extent of inhibition in this assay was calculated as % inhibition=(1−% migration/mean of % migration w/o antibody)*100.

To conduct the assay, three dilutions of antibodies, from 10 nM to 0.1 nM in triplicate, were prepared, for 6-hr incubation at 37° C. Cells were counted on Cytoflex, repeated twice (n=2) on M300.19 cells expressing the CXCR5 target antigen.

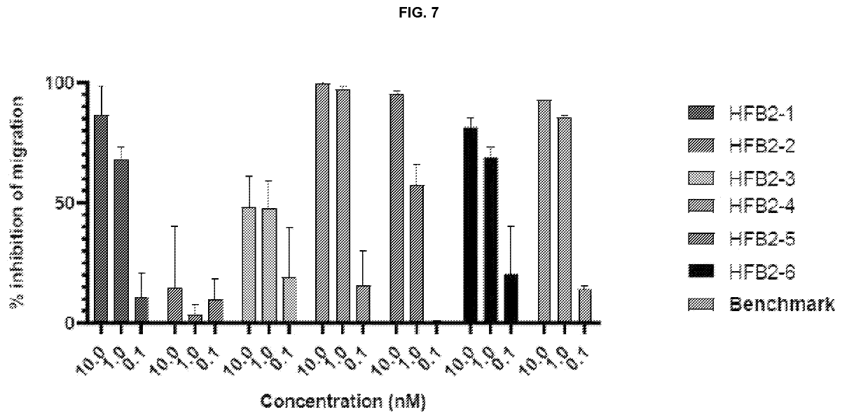

The results showed that the candidate antibodies showed different inhibition properties, and at least some of them were able to inhibit the ligand (CXCL13)-induced chemotaxis. The most potent antibody candidate to inhibit such chemotaxis was the chimeric antibody HFB2-4-hG1, which reached 100% inhibition at about 1 nM. See FIG. 7.

Additionally, strong binders were tested in cAMP assay in order to confirm their agonistic/antagonistic properties. It was assessed whether the candidate antibodies could block intracellular cAMP levels upon activation by CXCL13 ligand. The assay was based on the principle that a measurable chemiluminescent signal was directly proportional to the amount of cAMP in the cells. Upon an activation with ligand in Gαi pathway, a decrease of cAMP is expected. An increase of chemiluminescent signal is inversely correlated with cAMP levels.

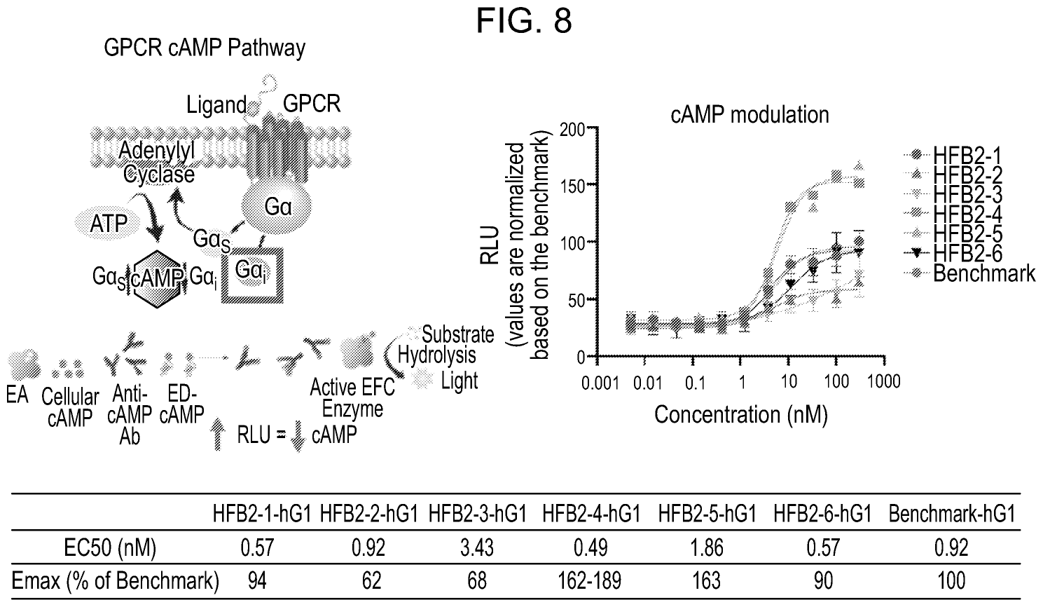

The cAMP assay showed different functional properties of the 6 candidate antibodies. Specifically, HFB2-4-hG1 and HFB2-5 efficiently blocked CXCL13-induced intracellular cAMP signaling as compared to the other 4 candidate antibodies, and both were superior to the benchmark antibody. FIG. 8.

Due to desired target product profile of the subject antibodies, the six strong chimeric binders were also tested in ADCC reporter bioassay.

ADCC is an immune mechanism through which Fc receptor-bearing effector cells can recognize and kill antibody (Ab)-coated target cells expressing antigens on their surface. ADCC is triggered by the cross-linking between antigen-bound Abs and the Fc receptor CD16A at the surface of immune effector cells. These interactions induce the increase of intracellular calcium concentrations, calcineurin/calmodulin-mediated dephosphorylation of NFAT, allowing its nuclear translocation and binding to promoter regions of ADCC relevant genes. Ultimately, the effector cells release cytotoxic granules which kill the target cells. For the reporter bioassay, the effector cells have been designed as effector reporter cells, in that these cells stably express a reporter gene such as a luciferase reporter gene under the control of a minimal promoter fused to multiple NFAT response elements. In this system, ADCC induction was measured as a bioluminescent signal produced by the luciferase upon activation of CD16A signaling by the antigen-bound antibody, and upon the addition of appropriate detection reagents.

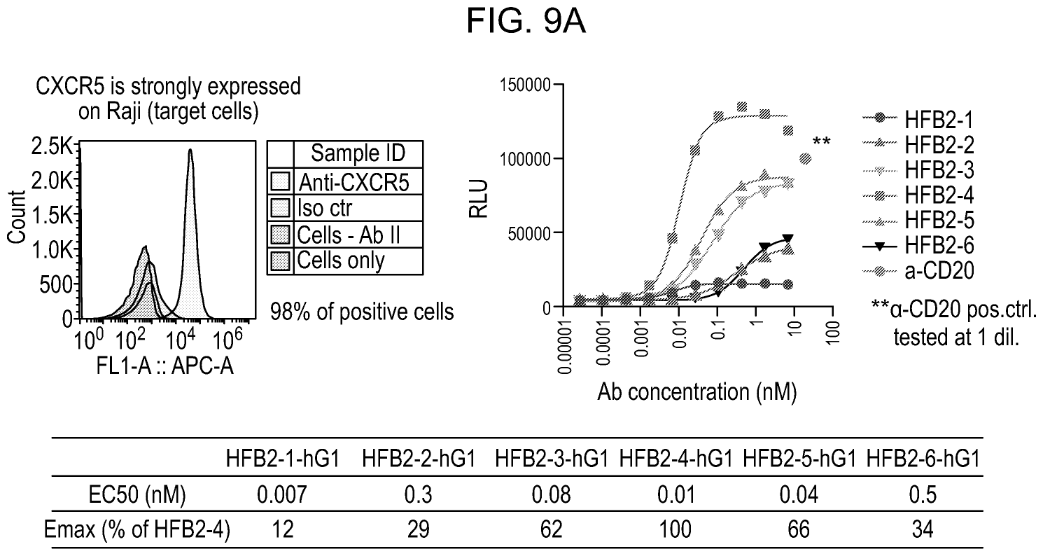

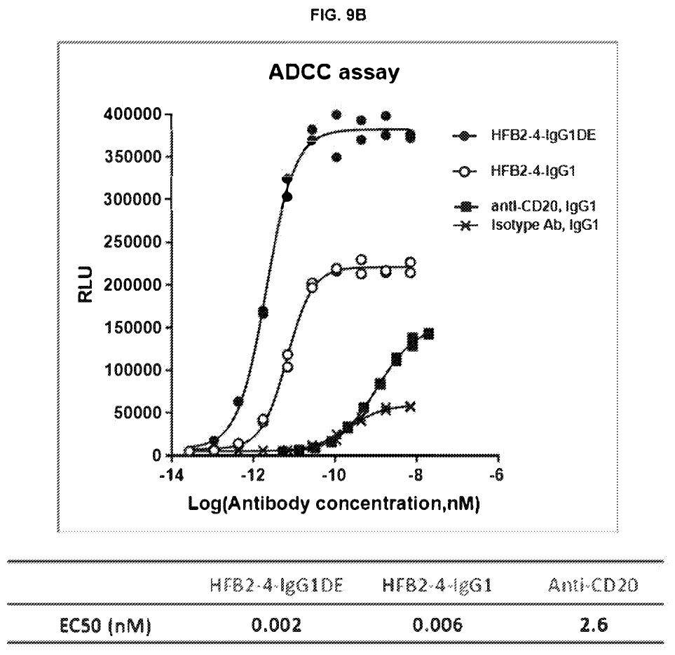

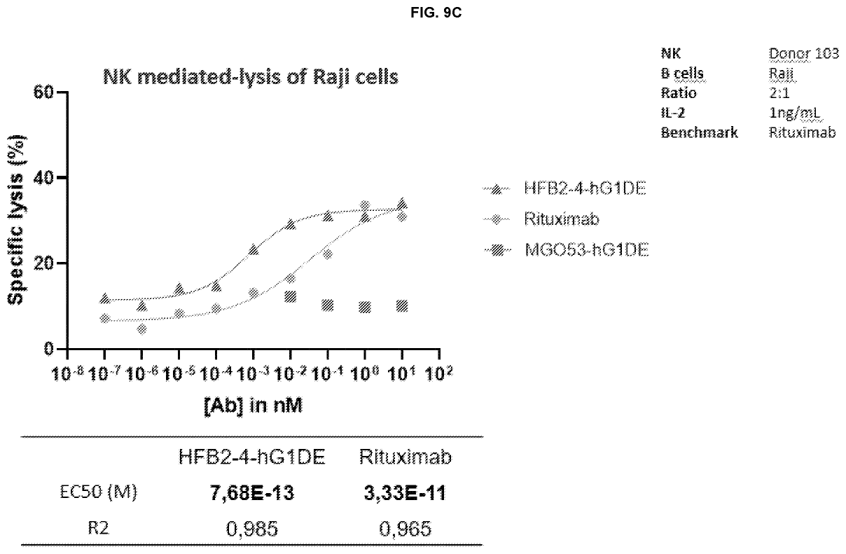

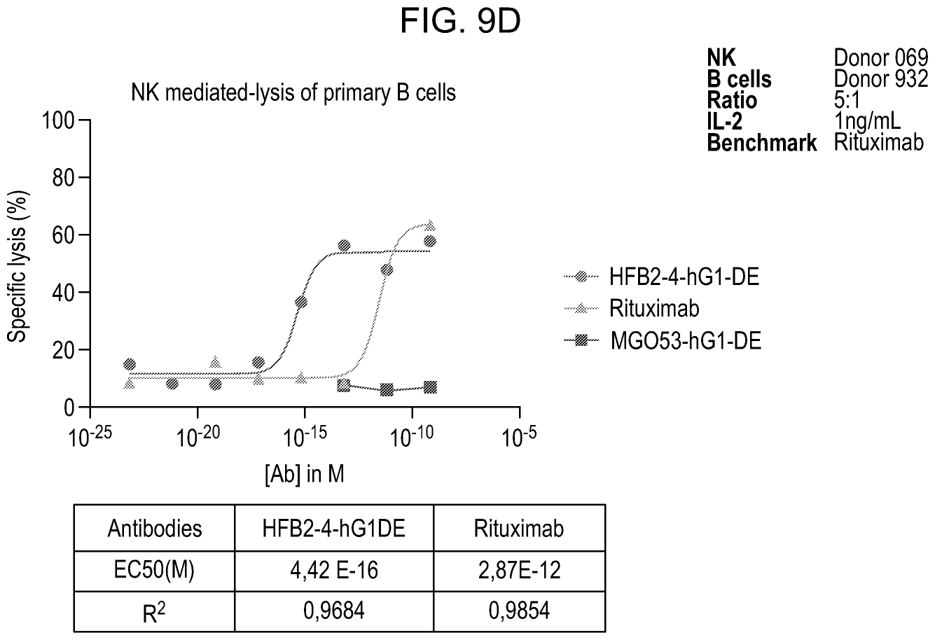

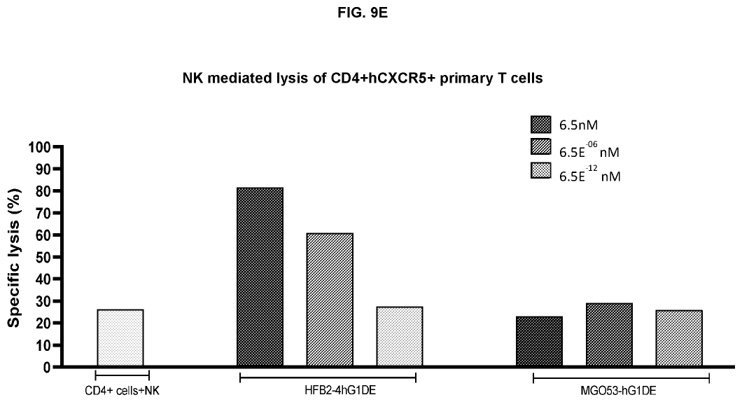

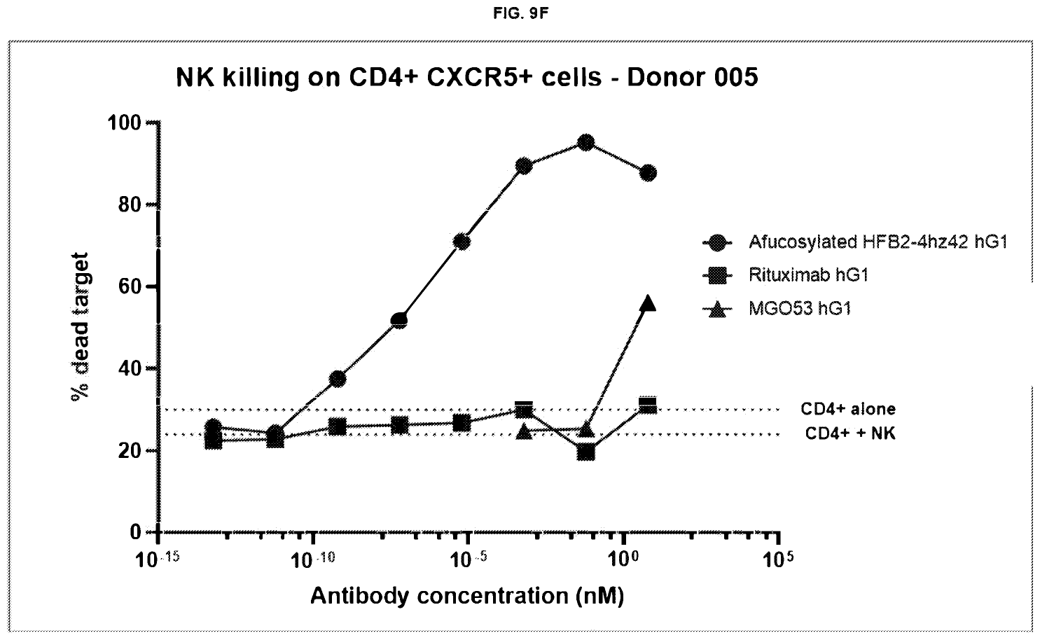

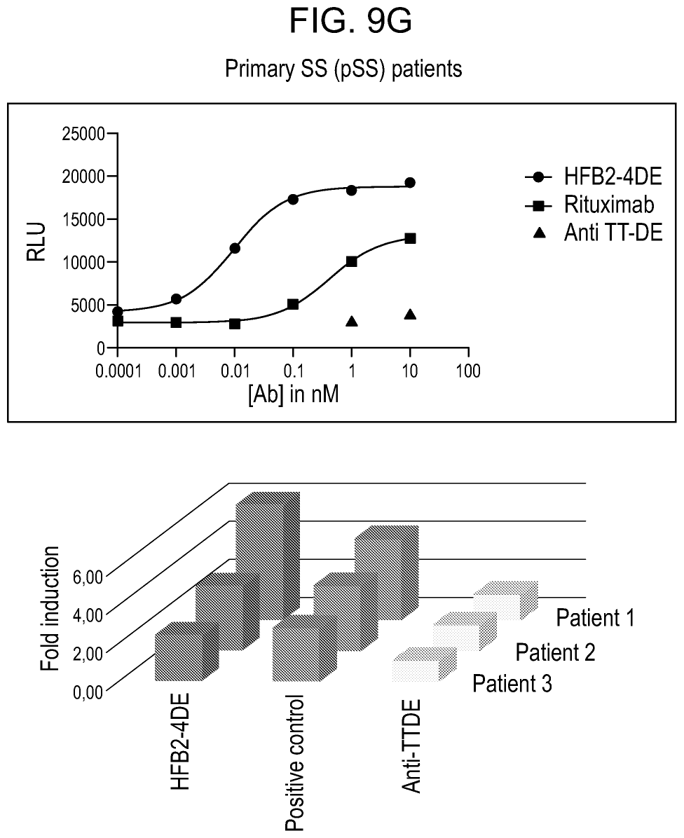

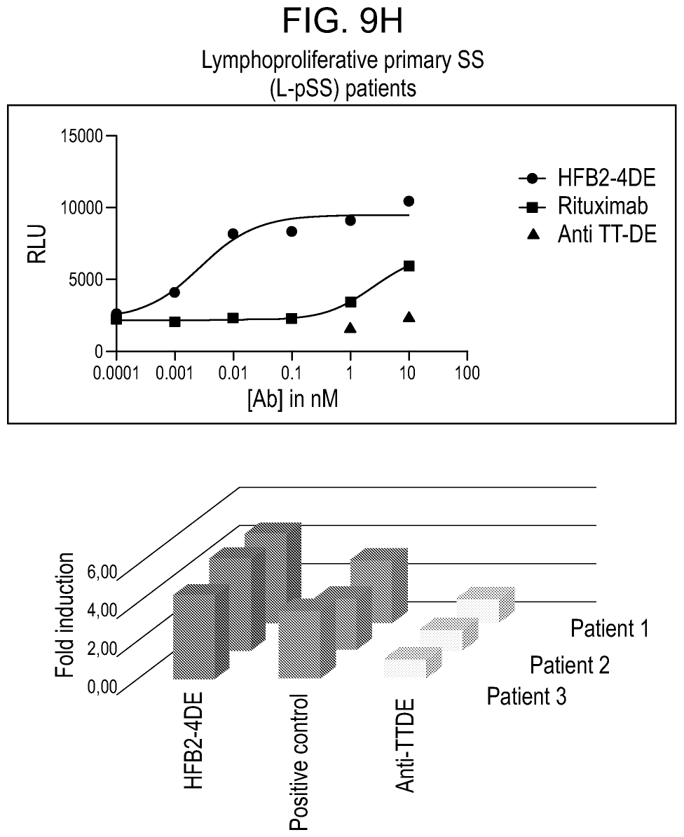

An anti-CD20 antibody, with already known mode of action, was used as positive control in this assay. Although different candidate antibodies showed clearly different ADCC capacities, HFB2-4-hG1 was shown to have the best potency in CD16 engagement. FIG. 9A.

In summary, six single-digit nanomolar affinity chimeric binders (i.e., HFB2-1-hG1, HFB2-2-hG1, HFB2-3-hG1, HFB2-4-hG1, HFB2-5-hG1, HFB2-6-hG1) out of 54 specific anti-CXCR5 antibodies were identified. The characterization of these six strongest binders showed various properties. None cross-reacted with murine or monkey ortholog of CXCR5. All of them are target-specific, and do not recognize the closest homolog CXCR3.

### Pharmacokinetic (PK) Evaluation of HFB2-4hG1

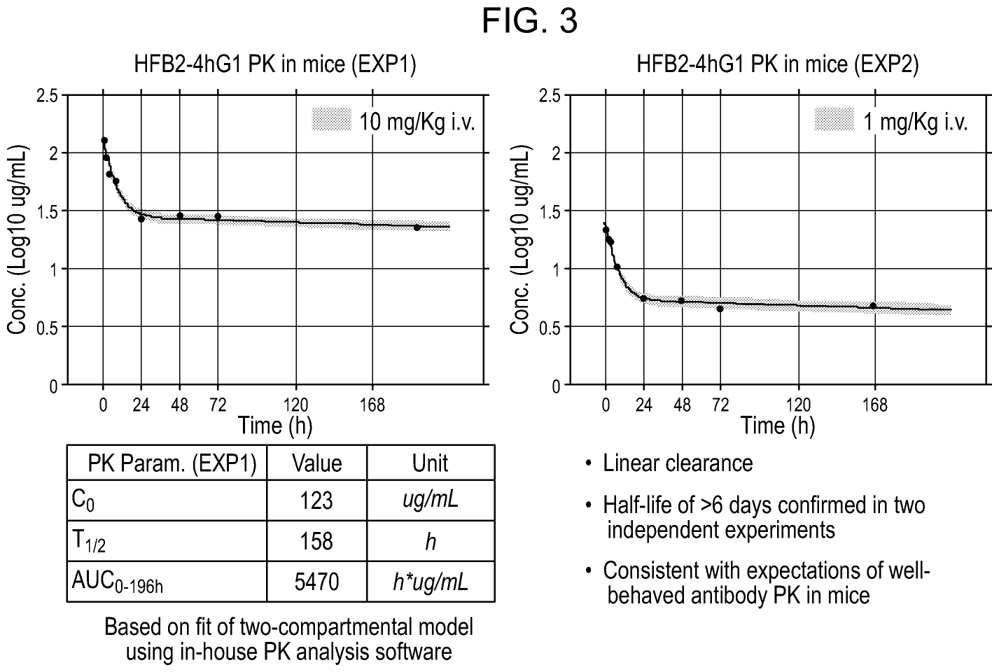

Based on the three different functional tests as cAMP, chemotaxis, and ADCC, HFB2-4hG1 appeared to be the most promising and potent candidate, and was selected for PK evaluation. See the favorable PK profile of the HFB2-4 hG1 chimeric antibody in mouse (FIG. 3).

A pharmacokinetic (PK) study of HFB2-4-hG1 was conducted in 8-10 weeks old wild-type (wt) C57BL/6J mice. HFB2-4-hG1 was intravenously administered through tail vein at two different concentrations as 1 mg/kg and 10 mg/kg. About 50 μL of plasma sample from each mouse at 8 different time points (at 1h, 2h, 4h, 8h, 24 h, 48h, 72h, and 7d) was collected. Human IgG levels in each sample was quantified by ELISA using a standard protocol internally optimized. The results showed that HFB2-4-hG1 demonstrated a favorable PK profile (t_{1/2}=158 hrs).

### Impact of DE Mutation on ADCC Activity of HFB2-4hG1

Given the fact that HFB2-4hG1 antibody showed the best CD16 engagement among other 5 strong hCXCR5 binders, HFB2-4hG1 antibody was selected to be produced in the DE scaffold (S239D/I332E) to enhance the ADCC activity. Both HFB2-4-hG1 and HFB2-4-hG1DE antibodies were tested in ADCC reporter bioassay including anti-CD20 positive control antibody. As expected, the results clearly confirmed that HFB2-4 induced a higher degree of CD16 engagement than the control anti-CD20 antibody, and showed that the DE scaffold increased the ADCC properties of the antibody further (in both Eₘₐₓ and EC₅₀), with a better activity than the positive control antibody anti-CD20. FIG. 9B.

As next, HFB2-4hG1DE was tested for its ADCC activity by using primary NK cells (effector cells) isolated from healthy donor peripheral blood mononuclear cells (PBMCs) and Raji cells (target cells) expressing the target. The data showed that HFB2-4hG1DE mediated lysis of Raji cells by primary NK cells with 0.76 pM EC50 value, which is more potent than Rituximab positive control antibody. See FIG. 9C.

Additionally, the ADCC activity of HFB2-4hG1DE was further tested for ADCC activity by using primary NK cells (effector cells) and primary B cells (target cells) expressing the target; both primary cell populations isolated from PBMCs of different healthy donors. HFB2-4hG1DE induced lysis of peripheral B cells by primary NK cells with 0.44 fM EC50 value, which is also more potent than Rituximab positive control antibody. See FIG. 9D. Similar experiments were also carried out to examine ADCC activity of HFB2-4hG1DE on lysis of human primary T cells, and HFB2-4hG1DE was found to also induce strong NK mediated-lysis of hCXCR5+ CD4+ primary T cells. See FIG. 9E.

A further similar experiment was carried out to examine ADCC activity of an afucosylated HFB2-4hG1 (no DE mutation) monoclonal antibody (AfuHFB2-4hG1) on lysis of human primary T cells. AfuHFB2-4hG1 was found to also induce strong NK mediated-lysis of hCXCR5+ CD4+ primary T cells, in a dose-dependent manner over a very wide range of antibody concentrations (see FIG. 9F), while Rituximab (hG1) (which targets B cells expressing the CD20 surface antigen), and the isotype control antibody MGO53-hG1 essentially had no effect over a wide range of antibody concentrations, as compared to the no antibody controls (CD4+ cells alone, with or without NK cells). See FIG. 9F.

### Anti-Tumor Activity In Vivo of HFB2-4hG1

Following the binding data obtained on cancer cell lines (Raji and Daudi cells), an in vivo study was conducted in CB17-SCID immunodeficient mice using only the Raji cell based tumor model. Briefly, Raji cells were inoculated subcutaneously in 6-8 weeks old CB-17 SCID mice. Four different groups; isotype control (MGO53-hG1), PBS control, positive control (HFB2-Rituximab-hG1) and lead candidate group (HFB2-4-hG1) were included in the study. The treatment constituted of intraperitoneal (i.p.) administration of one of the antibodies at 10 mg/kg, every three days for 21 days. Tumor growth and body weight of each mice were measured every three days.

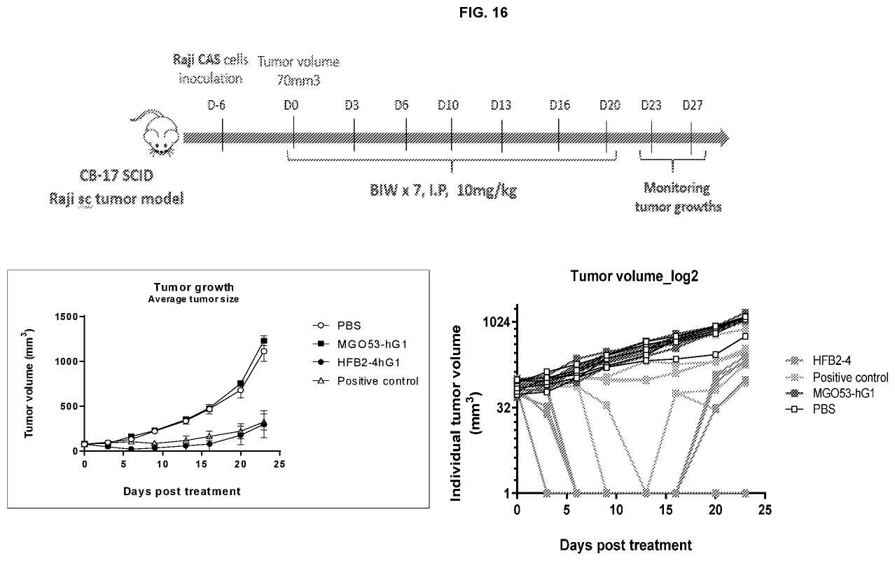

Administration of HFB2-4hG1 (10 mg/kg, BIW×7, i.p.) resulted in potent antitumor activity in Raji xenograft model comparable to Rituximab control. See FIG. 16.

In summary, HFB2-4 (hG1 and hG1DE) chimeric anti-hCXCR5 antibody showed strong binding to CXCR5 expressing cells, potently suppressed ligand-induced migration and signaling, demonstrated a favorable PK profile in mice, showed anti-tumor activity in Raji xenograft murine model study, mediated strong ADCC on primary NK cells against both Raji B cell lymphoma cell line and primary B cells. Based on the data generated in vitro and in e, the lead antibody series HFB2-4 (hG1 and hG1DE) demonstrated to be the most suitable candidate for humanization.

### Example 2 Characterization of Humanized Antibodies

Humanized anti-hCXCR5 monoclonal antibodies were generated based on the HFB2-4-hG1, using CDR-grafting, by selecting the Complementarity-Determining Regions (CDRs), responsible for antigen recognition of the antibody, from the murine antibody sequence and grafting them into the human Framework Region (FR) of hIgG1.

Overall, a total of 25 humanized variants were generated using CDR grafting. The majority of those variants preserved their physico-chemical (affinity to the target, stability, solubility) and/or biological activity (blockage or stimulation of the target, ADCC) compared to the parental chimeric antibody.

Briefly, 10 out of 12 humanized antibody variants based on HFB2-4 (i.e., HFB2-4 hz) showed comparable binding to hCXCR5 as the parental chimeric antibody HFB2-4. In addition, 9 out of 12 HFB2-4 hz variants blocked cAMP signaling, which results were consistent with the binding results. Furthermore, potent chemotaxis inhibition by the humanized variants was observed, with HFB2-4hz12 being the most potent (˜100% at 0.1 nM). In a subsequent experiment, comparable potency was also found between HFB2-4Hz9 and HFB2-4Hz12.

The humanized anti-hCXCR5 monoclonal antibodies were characterized further following the same procedure used for the in vitro characterization of chimeric monoclonal antibodies (see Example 1). In addition to critical and 2^{nd} order characterization assays, developability assessment tests were also included in humanized variant characterization.

### Assessment of Antibody Internalization

Antibody internalization by cells expressing antigen of interest (e.g., CXCR5) was assessed by Incucyte Antibody Internalization and pHAb reactive dyes (Promega) assays.

Antigen-mediated antibody internalization plays an important role in several antibody-based therapeutics. Depending on the desired mode of action, it may be desirable to have antibodies that are preferentially internalized into a target cell upon binding cell-surface antigen (such as delivery of highly toxic drugs to cancer cells via antibody drug conjugates (ADCs), and removal or degradation of surface receptors from cancer cells (i.e. EGFR)), or antibodies that preferentially remain bound to cell surface (such as identifying tumor cells for immune cell killing (i.e. ADCC or ADCP)). Additionally, measuring and optimizing functional responses to antibodies, such as antibody clearance, is of great importance. For example, pinocytosis, which is one of the main elimination routes of antibodies, requires antibody optimization for qualitative pharmacokinetic measurements during antibody development. As each approach requires a series of antibody features, for example, to enable maintenance on the cell surface for identification of tumor cells, or for rapid internalization when delivering ADC's, it is important to understand the uptake profile and clearance of antibody candidates for optimal antibody engineering and internalization characteristics.

The IncuCyte® FabFluor Antibody Labeling Reagents are Fc-region targeting Fab fragments conjugated to a pH-sensitive fluorescent probe. These reagents enable a generic one-step, no-wash, labeling protocol for all isotype matched, Fc-containing test antibodies. At pH 7.0, the Fab-Ab complex has little or no fluorescence. When labeled antibodies are added to cells, a fluorogenic signal is observed as the Fab-Ab complex is internalized and processed via acidic (pH 4.5-5.5) lysosomes and endosomes. The full time course of internalization can be visualized and automatically quantified using real-time live cell analysis.

pHAb Dyes are pH sensor dyes that have very low fluorescence at pH >7, and a dramatic increase in fluorescence as the pH of the solution becomes acidic. pHAb Dyes have excitation maxima (Ex) at 532 nm and emission maxima (Em) at 560 nm. pHAb Dyes are designed specifically for labeling antibody. For example, pHAb Amine Reactive Dye(a) has a succinimidyl ester group that reacts with primary amines available on the lysine amino acids on the antibodies.

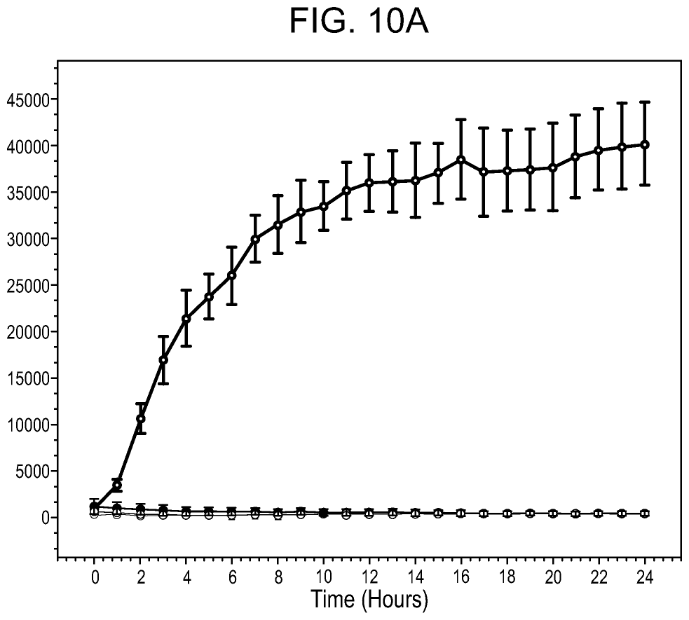

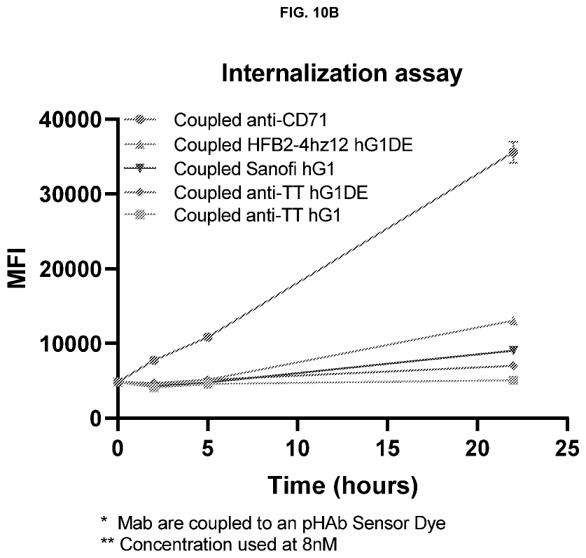

Results obtained from both assays showed that neither HFB2-4hz9-hG1DE nor HFB2-4hz12-hG1DE internalize as compared to the positive control (CD71) antibody. FIGS. 10A-10B.

### ADCC Activity of Humanized Variants

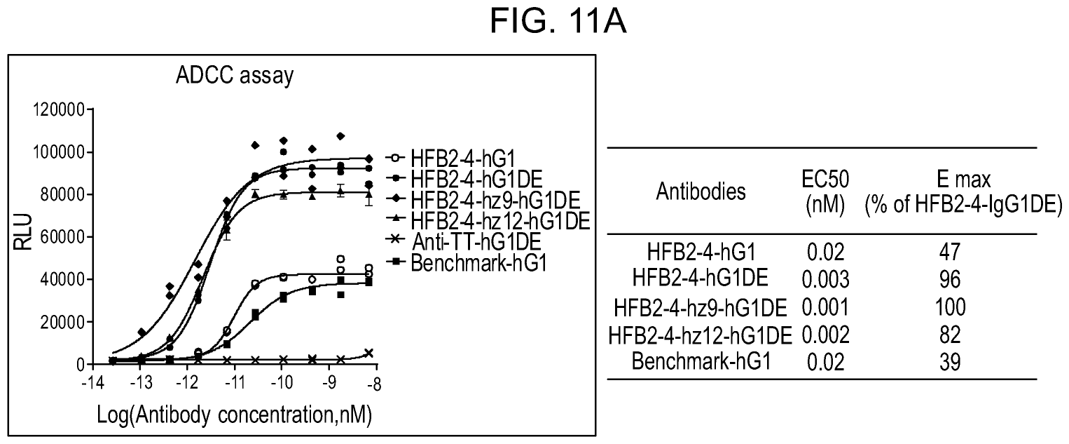

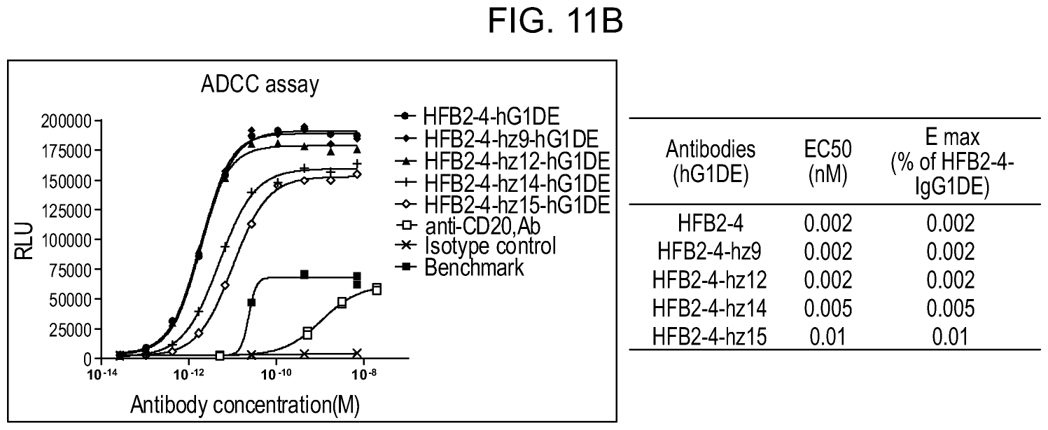

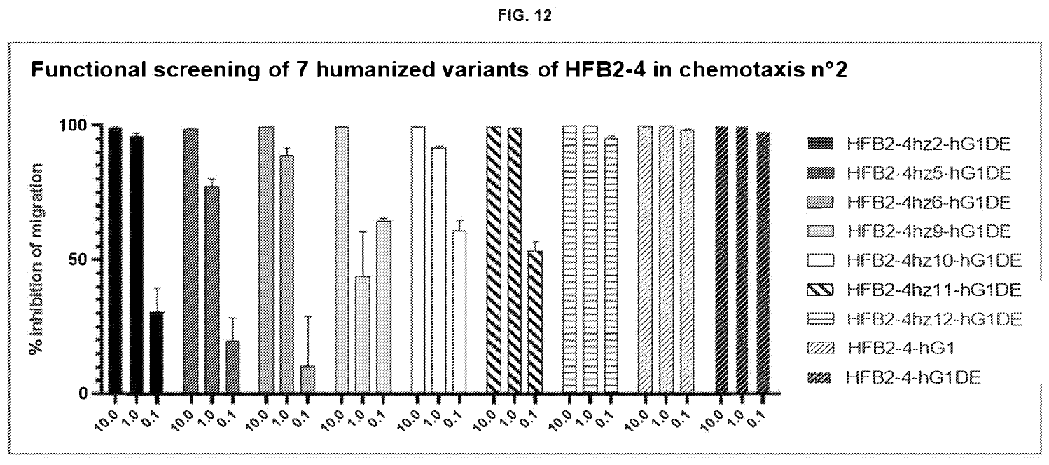

Two of the humanized antibodies, HFB2-4hz9-hG1DE and HFB2-4hz12-hG1DE, showed potent ADCC activity at the same extent as the parent chimeric antibody HFB2-4-hG1DE. As expected, HFB2-4 and benchmark antibodies in hG1 scaffold had lower ADCC activity as compared to the humanized variants. Results for two additional humanized variants, HFB2-4hz14-hG1DE and HFB2-4hz15-hG1DE, also showed an efficient engagement of CD16 to induce ADCC, as previously shown with HFB2-4hz9-hG1DE and HFB2-4hz12-hG1DE. See FIGS. 11A and 11B.

In order to optimize the assay conditions covering ADCC activity on Sjögren Syndrome patient samples, primary B cells isolated from peripheral blood mononuclear cells (PBMCs) from a healthy donor were used. Specifically, several healthy donors were screened for target antigen CXCR5 expression on primary B cells based on flow cytometry analysis. Donors with high primary B cell CXCR5 expression were used to optimize the conditions (as effector target ratio) of ADCC reporter bioassay. The Jurkat cells (Promega) were used as the reporter effector cells for measurement of the fluorescent signal emitted by its reporter luciferase activated by CD16-mediated signaling due to ADCC.

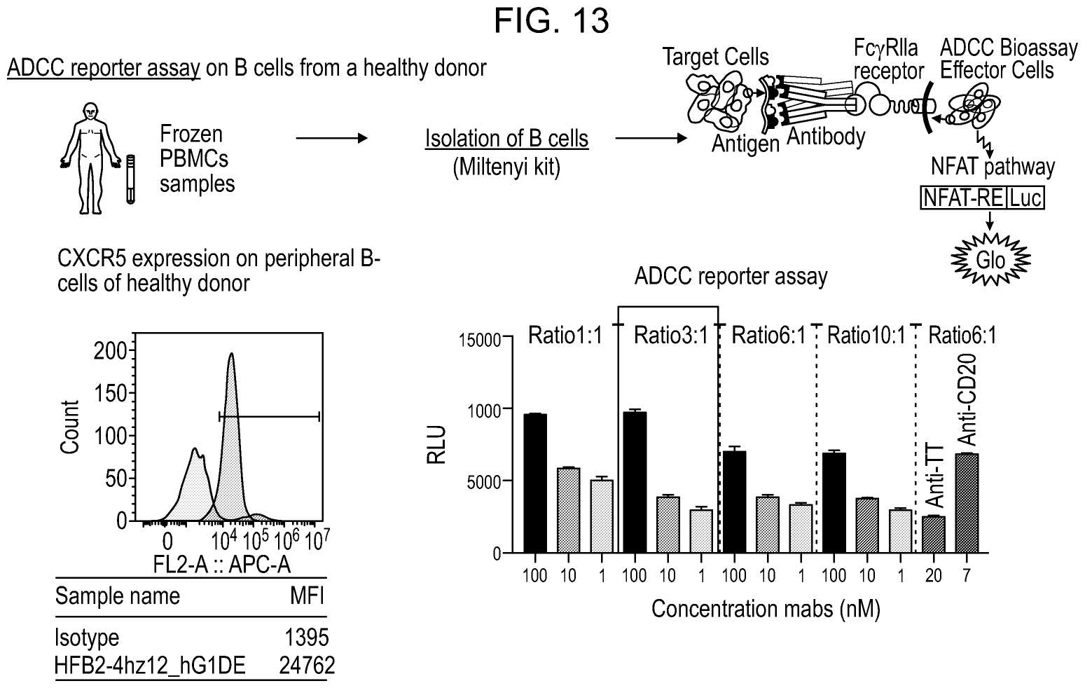

The results showed that both the parental chimeric antibodies as well as the humanized variants thereof demonstrated ADCC activity against primary B cells. See FIG. 13, which shows the results of the ADCC reporter assay for one of the humanized variants HFB2-4Hz12-hG1DE. The humanized antibody exhibited ADCC effect against CXCR5-expressing primary B cells.

### Anti-Tumor Activity In Vivo of a Humanized Variant

The ability of HFB2-4hz42 (in mouse IgG2a format) to induce anti-tumor activity was assessed in the in vivo intravenous Raji xenograft model. 8 mice per group were inoculated with Raji cells, and mice were treated with HFB2-4hz42 or isotype in mouse IgG2a format or Rituximab (hG1 format) as positive control. Additionally a group naive mice were with no treatment was also included. The mice were intraperitoneally injected with 10 mg/kg of the test antibody every three days for 15 days. As shown in the result table below, administration of HFB2-4hz42mIgG2 resulted in a significant increase in survival compared to mice treated with isotype control antibody.

| | Median | P value | P value |
|---|---|---|---|
| Group | survival | (comparison to G1) | (comparison to G3) |
| G1, MGO53-mG2a, | 22 | — | — |
| 10 mpk | | | |
| G2, HFB2-4hz42-mIgG2a, 10 mpk | 40 | <0.0001**** | 0.0023** |
| G3, Rituximab (hG1) | All survived | <0.0001**** | — |
| 10 mpk | | | |

### Example 3 Identification of Cancer Targets for the Humanized Antibodies

Prior to in vivo efficacy model selection, 8 different cancer cell lines were selected to be screened to confirm the in vitro target CXCR5 expression.

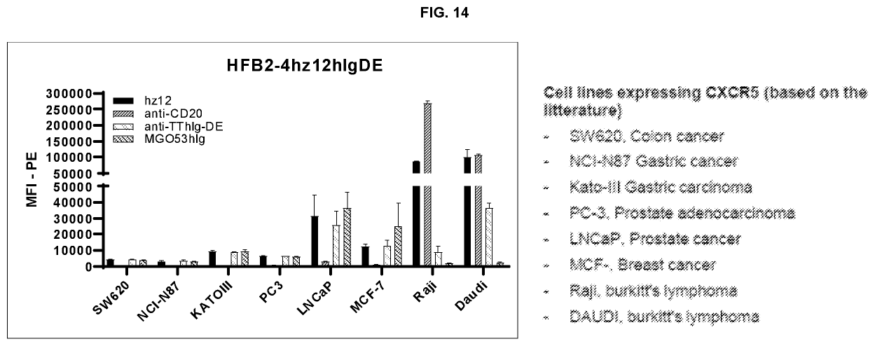

Binding assessment by flow cytometry demonstrated that HFB2-4hz12-hG1DE binds to Raji and Daudi cells in a similar extent than Rituximab—the anti-CD20 monoclonal antibody used to treat non-Hodgkin's lymphoma and chronic lymphocytic leukemia, but does not bind appreciably to any other selected cancer cell lines. See FIG. 14.

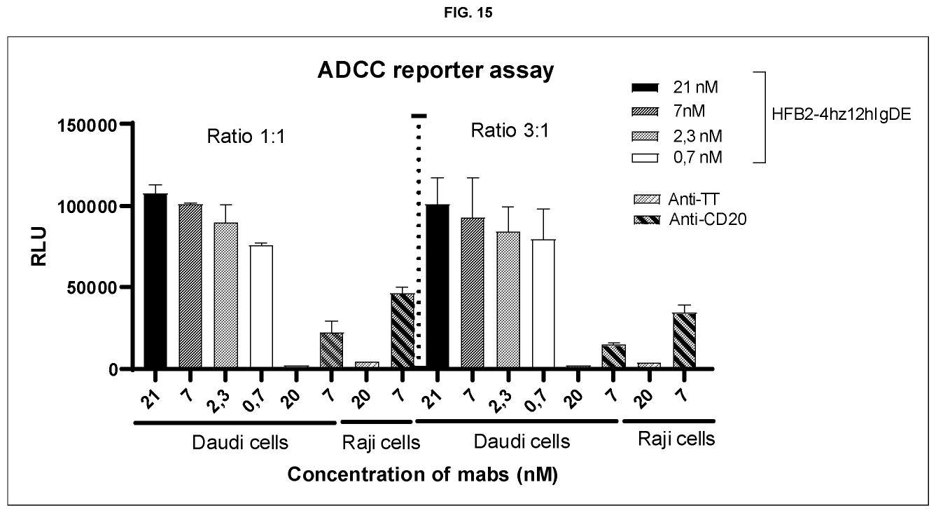

ADCC activity of this humanized antibody was then assessed on B cell lymphoma cell lines (Raji and Daudi cells), at different effector:target ratios (1:1 and 3:1). As before, in vitro characterization of chimeric and humanized variant antibodies was performed in ADCC reporter assay using Raji cells using already set experimental conditions, and only Daudi cells were tested in ADCC reporter assay. The data showed that HFB2-4hz12-hG1DE had ADCC activity against Daudi cells greater than the ADCC activity exhibited by the control anti-CD20 antibody. See FIG. 15.

### Example 4 ADCC Activity on Sjögren Patients' B Cells

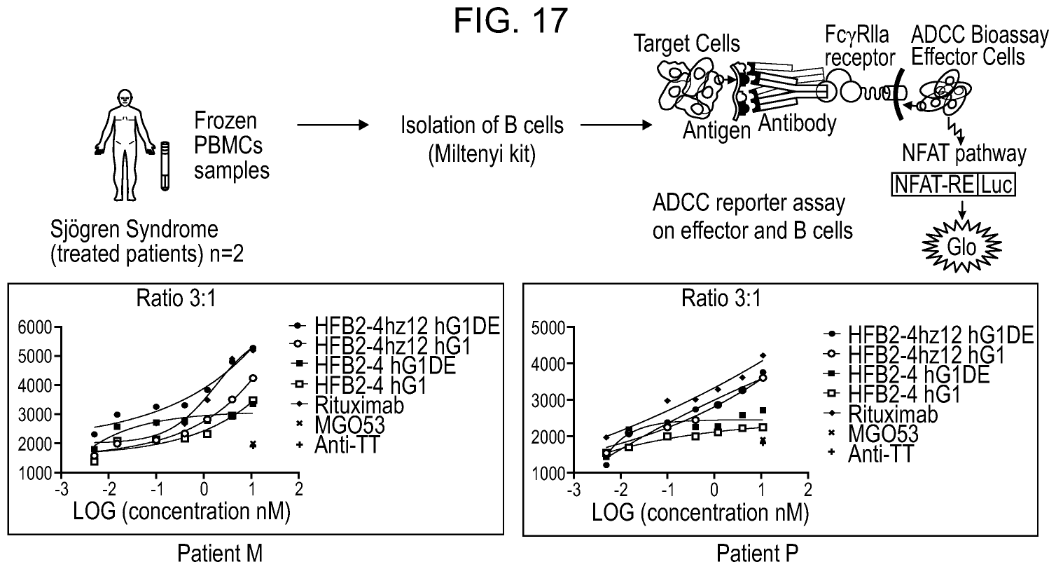

In order to demonstrate the potential efficacy of the subject antibodies in treating Sjögren syndrome (SS), ADCC reporter assay was used to show that a humanized monoclonal antibody of the invention, HFB2-4hz12hIgDE, can efficiently engages CD16 to induce ADCC effect on B cells isolated from frozen materials obtained from treated SS patients. See FIG. 17.

Specifically, B cells were isolated frozen PBMC samples obtained from 2 previously treated SS patients, and were used in the ADCC reporter assay as described above. Three-fold serial dilutions from 11 nM-0.005 nM for each antibody were used in the experiments. The results showed that HFB2-4hz12hIgDE efficiently engaged CD16 to induce ADCC.

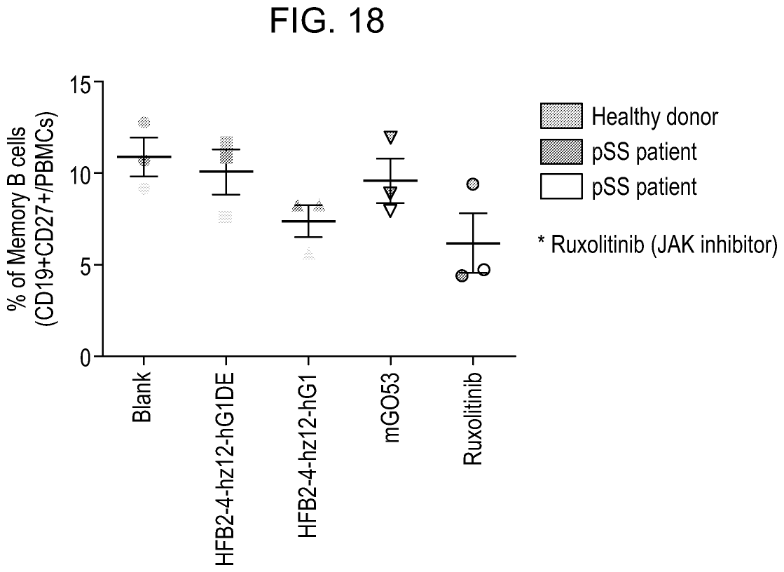

Additional data showed that HFB2-4hz12-hG1 reduced the percentage of memory B cell population in SS patient's samples. See FIG. 18.

As previously described, HFB2-4hG1DE was also tested on B cells from SS patients. using the ADCC reporter assay (Promega). It was observed that HFB2-4hG1DE had a higher CD16 engagement and induced a much stronger ADCC readout than rituximab in hG1 format. (FIGS. 9B-9C).

Overall, the results obtained so far suggested that humanized anti-hCXCR5 IgG1 antibody has nM affinity for human CXCR5, and induces ADCC in hCXCR5 expressing B cells, potentially follicular helper T cells/B cells in the germinal centers of Sjogren's Syndrome (SS) patients.

### Example 5 Generation and Characterization of Additional Series of Humanized Variations

By combining different variable heavy chains and variable light chains, 36 additional humanized variants were generated based on HFB2-4hG1 (HFB2-4 hz-hG1). These antibodies were characterized using methods similar to or as described in Example 1.

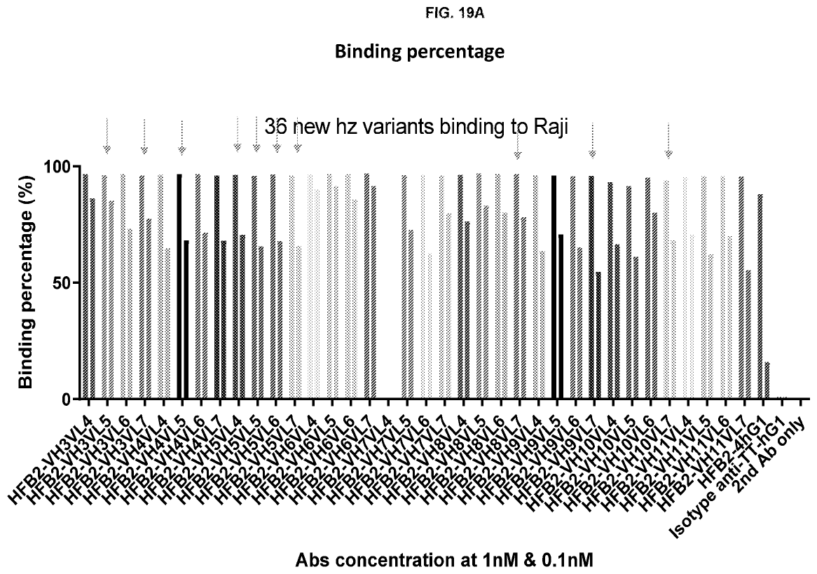

Binding properties of these 36 humanized variants were tested in binding assay to Raji cells at two different concentrations, 1 nM and 0.1 nM. All 36 humanized variants demonstrated significant binding profile to Raji cells. See FIGS. 19A and 19B.

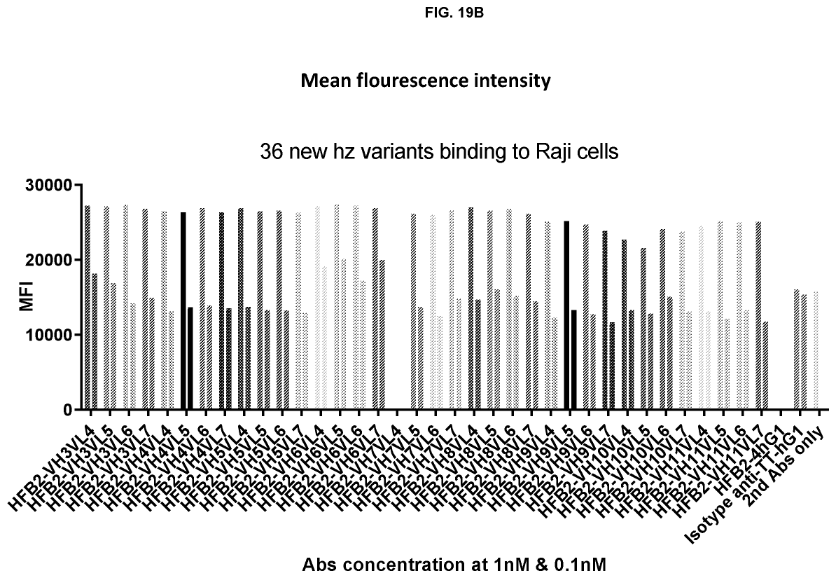

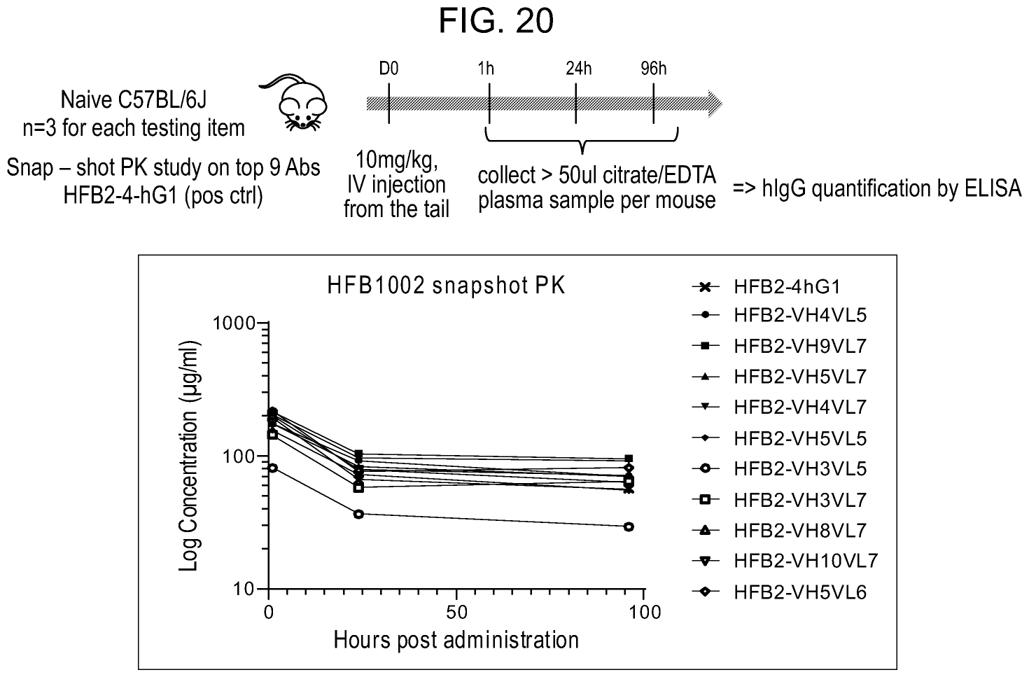

A pharmacokinetic study of top nine humanized variants was launched in 8 to 10 weeks old wild type C57BL/6J mice (n=3 for each humanized variant). Each humanized variant mAb was intravenously (i.v.) administered from the tail in a single dose at a concentration of 10 mg/kg. 50 μL of plasma sample from each mouse at four different time points as 1h, 24 h, 96h including pre-treatment dose at time 0 was collected. Human IgG levels in each sample was quantified by ELISA using a standard protocol internally optimized. All nine humanized variants demonstrated comparable PK profile to parental HFB2-4hG1. See FIG. 20. The top six humanized variants listed in Table 2 were selected for further in vitro and in vivo characterization.

TABLE 2
Top six humanized variants among the additional series based on HFB2-4hG1

| Abs Name | | Heavy | Light | VH | VL | |
|---|---|---|---|---|---|---|
| parental | | Chain name | Chain name | humanization | humanization | |
| antibody | HFB2-4hG1 | VH3 | VL4 | level % | level % | PI |
| HFB2-4hz37hG1 | HFB2-VH3VL6 | pHFB-HFB2-VH3 | pHFB-HFB2-VL6 | 87.40% | 80.60% | 8.65 |
| HFB2-4hz38hG1 | HFB2-VH3VL7 | pHFB-HFB2-VH3 | pHFB-HFB2-VL7 | | 83% | 8.5 |
| HFB2-4hz39hG1 | HFB2-VH6VL6 | pHFB-HFB2-VH6 | pHFB-HFB2-VL6 | 85.40% | 81% | 8.76 |
| HFB2-4hz41hG1 | HFB2-VH8VL7 | pHFB-HFB2-VH8 | pHFB-HFB2-VL7 | 78.10% | 83% | 8.5 |
| HFB2-4hz42hG1 | HFB2-VH10VL6 | pHFB-HFB2-VH10 | pHFB-HFB2-VL6 | 88.50% | 81% | 8.71 |
| HFB2-4hz45hG1 | HFB2-VH11VL7 | pHFB-HFB2-VH11 | pHFB-HFB2-VL7 | 78.10% | 83% | 8.5 |

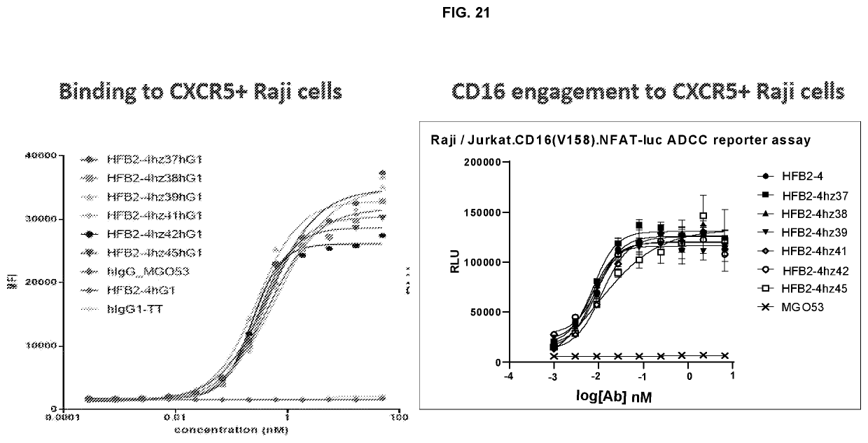

All top 6 HFB2-4 hz-hG1 variants exhibited similar binding profiles to Raji cells and ADCC activity to engage CD16 in reporter system. See FIG. 21.

Of note, among top 6 candidates, HFB2-4hz41hG1 and HFB2-4hz45hG1 demonstrated lower humanization level. Consequently, these two variants were excluded from the list, leaving 4 variants for further characterization.

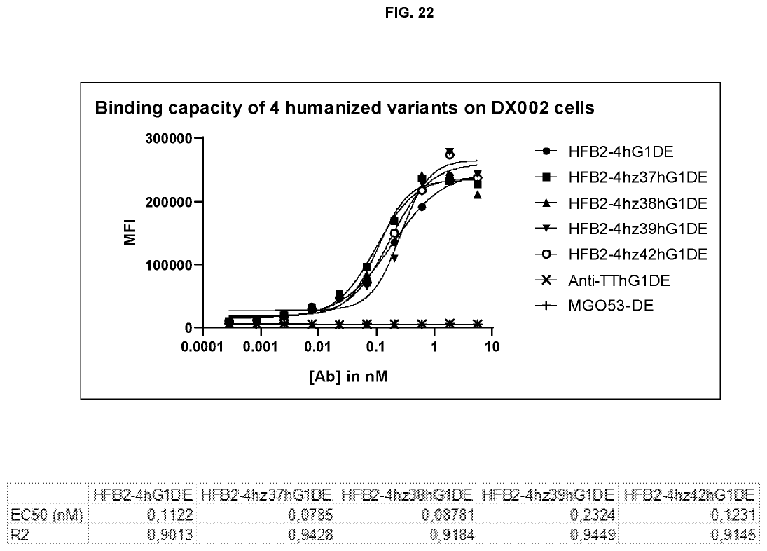

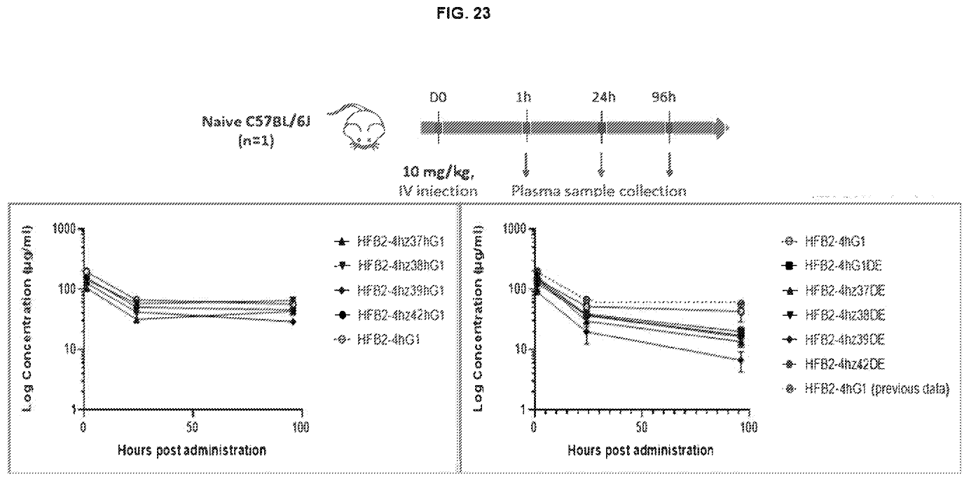

With the aim to enhance the ADCC activity of the best four humanized variants, these variants were produced in hG1DE format. The top four humanized variants in hG1DE format were tested for binding to adherent cells DX002-CHOK1 expressing the target CXCR5. The humanized variants showed comparable potency to HFB2-4hG1DE parental antibody. See FIG. 22.

To investigate whether the DE format impacts the pharmacokinetic (PK) profile of the antibodies, a snapshot PK study was launched in humanized variants in hG1 and hG1DE scaffold in 8 to 10 weeks-old wild type C57BL/6J mice (n=1 for each humanized variant). Each humanized variant antibody was intravenously administered from the tail in a single dose at a concentration of 10 mg/kg. 50 μL of plasma sample from each mouse at four different time points as 1h, 24 h, 96h including pre-treatment dose at time 0 was collected. Human IgG levels in each sample was quantified by ELISA using a standard protocol internally optimized. Humanized variants demonstrated comparable PK profile to parental antibody with the same format.

### Summary

Overall, 36 humanized variants were generated and tested. HFB2-4hz37hG1, HFB2-4hz38hG1, HFB2-4hz39hG1 and HFB2-4hz42hG1 were retained for the identification of lead candidate antibodies. The four humanized variants have a very similar profile to HFB2-4hG1-DE with comparable in vitro binding and biological properties.

However, in the PK analysis, HFB2-4hz39hG1 has a shorter half-life. Regarding developability profiles, HFB2-4hz42hG1 behaved slightly better. Regarding cIEF profiles, HFB2-4hz42hG1 and HFB2-4hz37hG1 showed the most suitable profiles. See Table 3.

Therefore, HFB2-4hz42hG1 and HFB2-4hz37hG1 are selected as lead and backup antibodies and will be further tested for in vivo efficacy study. The sequences of their VH and VL chains are shown in Table 4.

TABLE 3
Summary of characterizations of the top four humanized variants in hG1DE

| | | | Binding | CD16 | | | | Developability assessment study | | | | | |
|---|---|---|---|---|---|---|---|---|---|---|---|---|---|
| | VH | VL | to DX002 | engagement | Snapshot PK profile | | | | | | | Stress | Freeze/ |
| | Humaniz | humaniz | EC50 | EC50 | 1 h | 24 h | 96 h | | | Heat treatment | | at low pH | thaw |
| Ab Name | % | % | (nM) | (nM) | ug/ml | ug/ml | ug/ml | Formul. | cIEF | 25° C. | 40° C. | (6 h) | (5x) |
| HFB2-4hG1 | | | | 0.010 | 146 | 52 | 43 | | | | | | |
| HFB2-4hG1DE | | | 0.186 | | 138 | 39 | 20 | | | | | | |
| HFB2- | 87.4% | 80.6% | 0.099 | 0.010 | 151 | 30 | 14 | Acetate | 9.10 | STABLE | Degra- | Degra- | STABLE |
| 4hz37hG1DE | | | | | | | | | +++ | | dation | dation | |
| HFB2- | | 83.0% | 0.106 | 0.010 | 161 | 37 | 17 | Acetate | 8.80 | Degra- | Degra- | Degra- | STABLE |
| 4hz38hG1DE | | | | | | | | | ++ | dation | dation | dation | |
| HFB2- | 85.4% | 81.0% | 0.260 | 0.011 | 90 | 20 | 7 | Acetate | 9.35 | STABLE | Degra- | Degra- | STABLE |
| 4hz39hG1DE | | | | | | | | | ++ | | dation | dation | |
| HFB2- | 88.5% | 81.0% | 0.171 | 0.009 | 126 | 37 | 16 | Acetate | 9.15 | STABLE | Degra- | STABLE | STABLE |
| 4hz42hG1DE | | | | | | | | | +++ | | dation | | |

| | | | | |
|---|---|---|---|---|
| Compa- | Hz38, | Hz37, | Hz42 | Compa- |
| rable | Hz42, Hz37 | Hz39, Hz42 | | rable |

TABLE 4
VH and VL sequences of lead antibodies from the additional series
of antibodies based on HFB2-4hG1

| | | HC | LC | Variable HC | Variable LC |
|---|---|---|---|---|---|
| | Ab Name | name | name | amino acids | amino acids |
| | HFB2- | | | QVQLKESGPGLVAPS | DIQMTQSPSSLSASVGDRVTI |
| | 4hG1 | | | QSLSITCTVSGFSLT | TCRSSKSLLHSNGKTYLYWFQ |
| | (VH+VL) | | | SYGVSWVRQPPGKGL | QKPGKAPKLLIYRMSNLASGV |
| | | | | EWLGVIWGDGSTNYH | PSRFSGSGSGTDFTLTISSLQ |
| | | | | SGLISRLSISKDNSK | PEDFATYYCMQHLEYPYTFGG |
| | | | | SQVFLKLNSLQSDDT | GTKVEIK |
| | | | | ATYYCARVVYWGQGT | (SEQ ID NO: 111) |
| | | | | LVTVSA (SEQ ID | |
| | | | | NO: 56) | |
| HFB2- | HFB2- | pHFB- | pHFB- | QVQLQESGPGLVKPS | EIVMTQSPATLSVSPGERATL |
| 4hz37hG1 | VH3VL6 | HFB2- | HFB2- | ETLSLTCTVS | SCRSSKSLLHSNGKTYLYWFQQ |
| | | VH3 | VL6 | GFSLTSYGVSWVRQP | KPGQAPRLLIYRMSNLASGIP |
| | | | | PGKGLEWIG | ARFSGSGSGTEFTLTISSVQS |
| | | | | VIWGDGSTNYNPSL | EDVAVYYCMQHLEYPYTFGQG |
| | | | | KSRVTISKDTSKNQV | TKLEIK |
| | | | | SLKLSSVTAADTAVY | (SEQ ID NO: 112) |
| | | | | YCARVVYWGQGTLVT | |
| | | | | VSS (SEQ ID | |
| | | | | NO: 96) | |
| HFB2- | HFB2- | pHFB- | pHFB- | QVQLVQSGAEVKKPG | EIVMTQSPATLSVSPGERATL |
| 4hz42hG1 | VH10VL6 | HFB2- | HFB2- | ASVKVSCKAS | SCRSSKSLLHSNGKTYLYWFQQ |
| | | VH10 | VL6 | GFSLTSYGVS | KPGQAPRLLIYRMSNLASGIP |
| | | | | YAQKLQGRVTMTKDT | ARFSGSGSGTEFTLTISSVQS |
| | | | | STSTAYMELRSLRSD | EDVAVYYCMQHLEYPYTFGQG |
| | | | | DTAVYYCARVVYWGQ | TKLEIK (SEQ ID NO: |
| | | | | GTLVTVSS (SEQ | 112) |
| | | | | ID NO: 113) | |

*CDR sequences are double-underlined.

### Example 6 Generation and Characterization of Afucosylated Anti-CXCR5 Antibody

The afucosylated format of HFB2-4hG1 and HFB2-4hz42-hG1 to increase binding to FcγRIIIa and enhance antibody effector function were generated for testing using similar assays to those previously described. The results are summarized below.

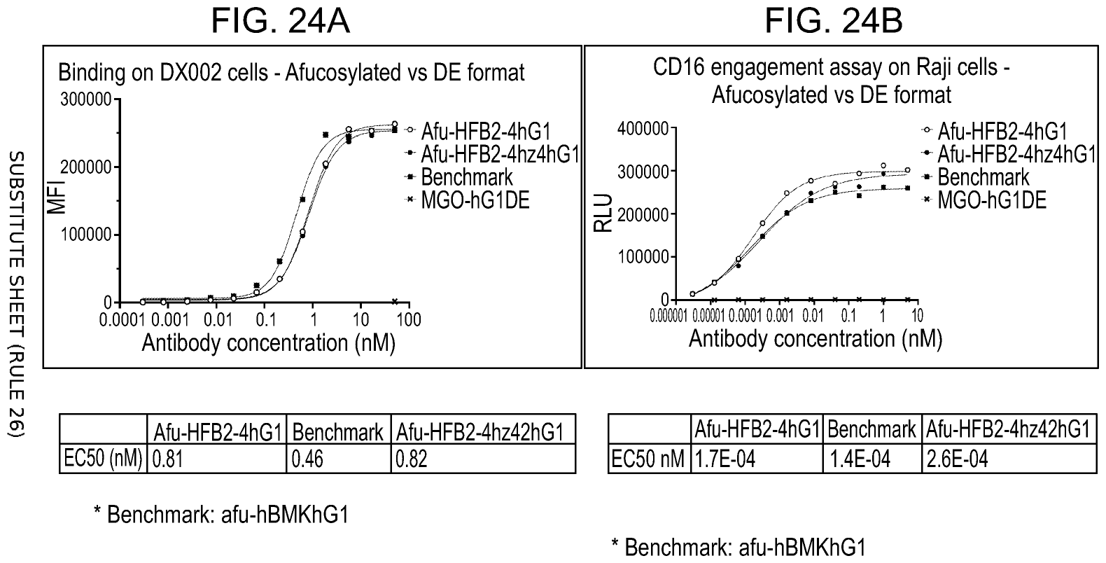

Afucosylated HFB2-4hG1 (afu-HFB2-4hG1) and HFB2-4hz42-hG1 (afu-HFB2-4hz42-hG1) binds to human CXCR5 on DX002 cells with sub-nM avidity and demonstrate similar binding profile to each other. See FIG. 24A.

CD16 engagement determined by the ADCC reporter assay also shows that afu-HFB2-4hz42-hG1 demonstrates similar potency to engage CD16 to afu-HFB2-4hG1 parental antibody. See FIG. 24B.

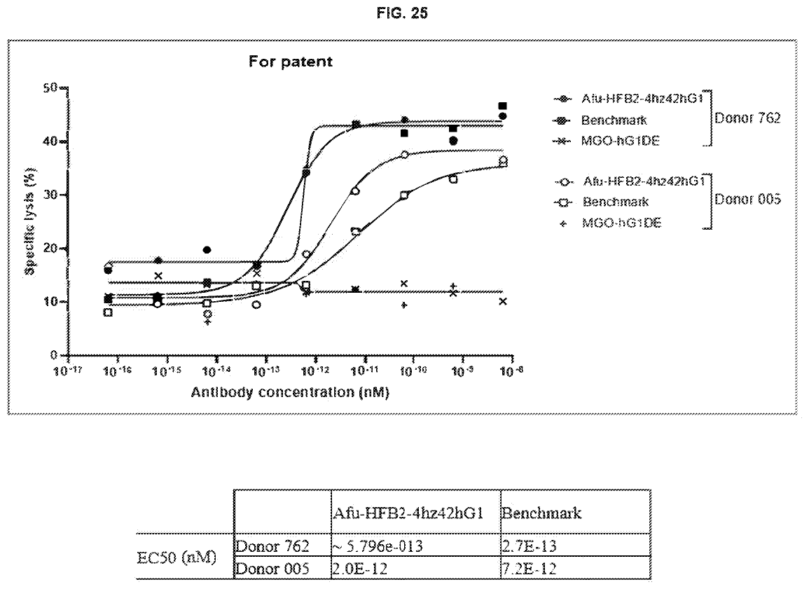

Additionally, in vitro killing of Raji cells (data not shown) and B cells isolated from healthy donors (FIG. 25) by NK cells through ADCC induced by afu-HFB2-4hz42-hG1 was comparable to benchmark antibody. Preliminary data on ADCC killing of T-follicular helper cells (Tfh) expressing CXCR5 also suggests that afu-HFB2-4hz42-hG1 can also induce NK-mediated lysis of CD4+CXCR5+ primary T cells isolated from human (data not shown).

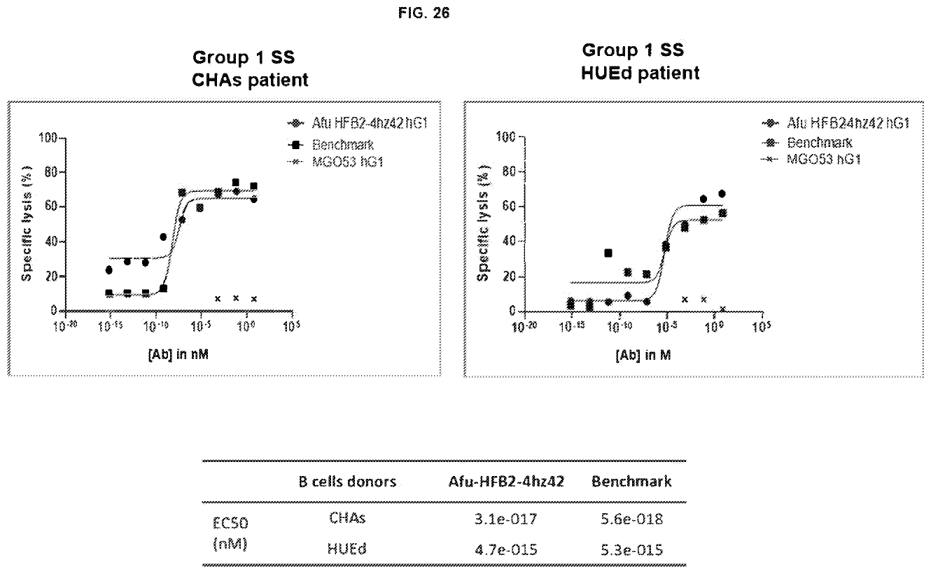

NK killing of B cells from SS patients was examined using afucosylated antibody. As shown in FIG. 26, afucosylated HFB2-4hz42-hG1 induced B cell lysis in a comparable degree to the benchmark antibody.

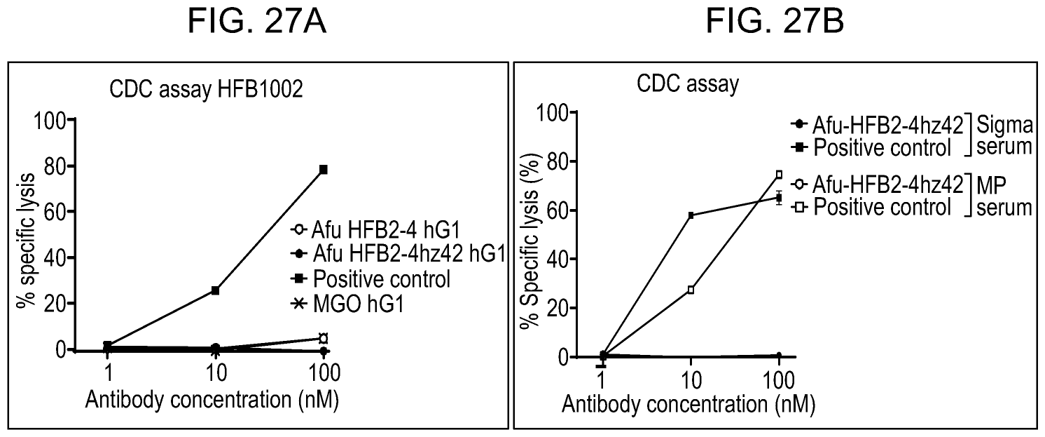

Furthermore, the afucosylated antibodies were tested for complement-dependent cytotoxicity (CDC). Rituximab (hIgG1 format), which is known to have CDC activity, was used as a positive control. Serum was used to provide components of the complement system in the experiment. Rituximab reaches ˜70-80% cell lysis at 100 nM, but no CDC activity was observed for afucosylated HFB2-4hz42-hG1 and afucosylated HFB2-4hG1. See FIGS. 27A-27B.

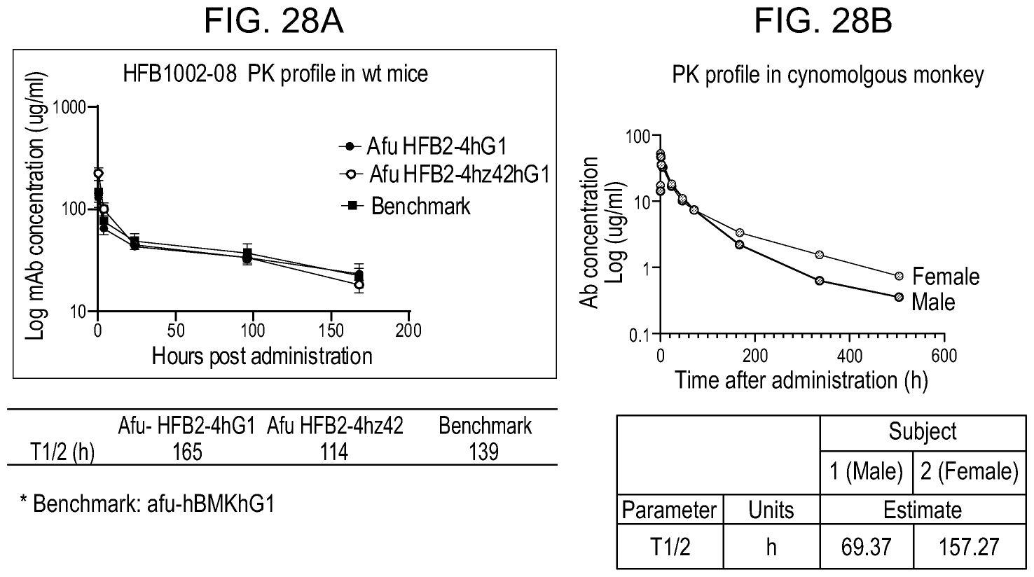

Pharmacokinetics of the afucosylated antibodies was examined in wild-type mice and cynomolgous monkey. In wild type C57BL/6J mice, afucosylated HFB2-4hz42-hG1, HFB2-4hG1 and benchmark antibodies were injected intravenously from the tail in a single dose at 10 mg/kg dose, and plasma was collected at 0.5, 6, 24, 96 and 168 hours post injection to determine antibody concentration. Half-life time of afu-HFB2-4hz42-hG1 was 114 hours, with PK profile similar to the parental and benchmark antibody. See FIG. 28A.

In cynomolgous monkey (1 male, 2 female), a single dose of antibody at 1 mg/kg was intravenously infused, and concentration of the antibody in blood was determined during 2 weeks after injection. Afu-HFB2-4hz42-hG1 had a half-life of 3-6.5 days in the tested monkey. See FIG. 28B.

Afucosylated HFB2-4hz42-hG1 was subjected to multiple stability tests: heat treatment at 25° C. and 40° C. up to 30 days, stress at low pH of 3.5 for up to 6 hours, and up to 5 freeze/thaw cycles. The results indicate that afucosylated HFB2-4hz42-hG1 is generally stable under these testing conditions (data not shown).

Overall, afucosylated HFB2-4hz42-hG1 exhibits great binding and in vitro ADCC activity against primary B cells from both health donors and SS patients at pM concentrations similar to the parental antibody, does not induce complement dependent cytotoxicity, displays favorable pharmacokinetic profiles and high stability. Therefore, afucosylated HFB2-4hz42-hG1 has great potential for use in clinical treatment.

### REFERENCES

All references discussed herein are incorporated by reference herein.
Förster R, Follicular B Helper T Cells Express Cxc Chemokine Receptor 5, Localize to B Cell Follicles, and Support Immunoglobulin Production, J Exp Med. 2000.
Allen C D, Germinal center dark and light zone organization is mediated by CXCR4 and CXCR5, Nat Immunol. 2004.
Förster R, A Putative Chemokine Receptor, BLR1, Directs B Cell Migration to Defined Lymphoid Organs and Specific Anatomic Compartments of the Spleen, Cell, 1996.
Reif K, Balanced responsiveness to chemoattractants from adjacent zones determines B-cell position, Nature, 2002.
Aqrawi L, Diminished CXCR5 expression in peripheral blood of patients with Sjögren's syndrome may relate to both genotype and salivary gland homing. Clinic Exp Immunol, 2018.
Szabo K, The Histopathology of Labial Salivary Glands in Primary Sjögren's Syndrome: Focusing on Follicular Helper T Cells in the Inflammatory Infiltrates, Mediators Inflamm. 2014.
Kramer J M, CXCL13 is elevated in Sjögren's syndrome in mice and humans and is implicated in disease pathogenesis, J Leukoc Biol, 2013.
Sharma A, Early BAFF receptor blockade mitigates murine Sjögren's syndrome: Concomitant targeting of CXCL13 and the BAFF receptor prevents salivary hypofunction, Clin Immunol, 2016.
Moser B, CXCR5, the defining marker for follicular B helper T (TFH) cells, Front Immunol, 2015.

1. An isolated monoclonal antibody, or an antigen-binding fragment thereof, wherein said monoclonal antibody or antigen-binding fragment thereof is specific for human CXCR5, and wherein said monoclonal antibody comprises:
(1) a heavy chain variable region (VH), comprising a VH CDR1 sequence, a VH CDR2 sequence, and a VH CDR3 sequence; wherein said VH CDR1 sequence, said VH CDR2 sequence, and said VH CDR3 sequence comprise any one of the VH CDR1, VH CDR2, and VH CDR3 sequences, respectively, in Tables A, B, and D;
optionally, said VH CDR1 sequence, said VH CDR2 sequence, and said VH CDR3 sequence comprise the VH CDR1, VH CDR2, and VH CDR3 sequences, respectively, of any one of the monoclonal antibodies in Tables A, B, and D; and/or
(2) a light chain variable region (VL), comprising a VL CDR1 sequence, a VL CDR2 sequence, and a VL CDR3 sequence; wherein said VL CDR1 sequence, said VL CDR2 sequence, and said VL CDR3 sequence comprise any one of the VL CDR1, VL CDR2, and VL CDR3 sequences, respectively, in Tables A, C, and D; optionally, said VL CDR1 sequence, said VL CDR2 sequence, and said VL CDR3 sequence comprise the VL CDR1, VL CDR2, and VL CDR3 sequences, respectively, of any one of the monoclonal antibodies in Tables A, C, and D.

2. The isolated monoclonal antibody or antigen-binding fragment thereof of claim 1, wherein:
(1) the VH CDR1 sequence, the VH CDR2 sequence, and the VH CDR3 sequence comprise the amino acid sequences of SEQ ID NOs: 1, 2, and 3, respectively, and the VL CDR1 sequence, the VL CDR2 sequence, and the VL CDR3 sequence comprise the amino acid sequences of SEQ ID NOs: 9, 10, and 11 respectively;
(2) the VH CDR1 sequence, the VH CDR2 sequence, and the VH CDR3 sequence comprise the amino acid sequences of SEQ ID NOs: 17, 18, and 19, respectively, and the VL CDR1 sequence, the VL CDR2 sequence, and the VL CDR3 sequence comprise the amino acid sequences of SEQ ID NOs: 25, 26, and 27 respectively;
(3) the VH CDR1 sequence, the VH CDR2 sequence, and the VH CDR3 sequence comprise the amino acid sequences of SEQ ID NOs: 33, 34, and 35, respectively, and the VL CDR1 sequence, the VL CDR2 sequence, and the VL CDR3 sequence comprise the amino acid sequences of SEQ ID NOs: 41, 42, and 43 respectively;
(4) the VH CDR1 sequence, the VH CDR2 sequence, and the VH CDR3 sequence comprise the amino acid sequences of SEQ ID NOs: 33, 49, and 51, respectively, and the VL CDR1 sequence, the VL CDR2 sequence, and the VL CDR3 sequence comprise the amino acid sequences of SEQ ID NOs: 57, 58, and 59 respectively;
(5) the VH CDR1 sequence, the VH CDR2 sequence, and the VH CDR3 sequence comprises the amino acid sequence of SEQ ID NOs: 33, 49, and 65, respectively, and the VL CDR1 sequence, the VL CDR2 sequence, and the VL CDR3 sequence comprises the amino acid sequence of SEQ ID NOs: 57, 58, and 59 respectively;
(6) the VH CDR1 sequence, the VH CDR2 sequence, and the VH CDR3 sequence comprise the amino acid sequences of SEQ ID NOs: 69, 70, and 71, respectively, and the VL CDR1 sequence, the VL CDR2 sequence, and the VL CDR3 sequence comprise the amino acid sequences of SEQ ID NOs: 76, 77, and 78 respectively;
(7) the VH CDR1 sequence, the VH CDR2 sequence, and the VH CDR3 sequence comprise the amino acid sequences of SEQ ID NOs: 33, 49, and 51, respectively, and the VL CDR1 sequence, the VL CDR2 sequence, and the VL CDR3 sequence comprise the amino acid sequences of SEQ ID NOs: 149, 150 and 151, respectively; or
(8) the VH CDR1 sequence, the VH CDR2 sequence, and the VH CDR3 sequence comprise the amino acid sequences of SEQ ID NOs: 114, 115, and 116, respectively, and the VL CDR1 sequence, the VL CDR2 sequence, and the VL CDR3 sequence comprise the amino acid sequences of SEQ ID NOs: 120, 121, and 122 respectively.

3. The isolated monoclonal antibody or antigen-binding fragment thereof according to claim 1 or 2, wherein said monoclonal antibody is a mouse-human chimeric antibody comprising constant region sequences of a human antibody (such as hIgG1, hIgG2, hIgG3, or hIgG4), wherein the VH sequence is any one of SEQ ID NOs: 8, 24, 40, 56, 65 or 75 or has at least 90%, 91%, 92%, 93%, 94%, 95%, 96%, 97%, 98%, 99% sequence identity to any one of the amino acid sequences of SEQ ID NOs: 8, 24, 40, 56, 65 or 75, and/or wherein the VL sequence is any one of SEQ ID NOs: 16, 32, 48, 63, 68 or 83 or has at least 90%, 91%, 92%, 93%, 94%, 95%, 96%, 97%, 98%, 99% sequence identity to any one of the amino acid sequences of SEQ ID NOs: 16, 32, 48, 63, 68 or 83.

4. The isolated monoclonal antibody or antigen-binding fragment thereof according to claim 3, wherein:
(1) the VH sequence is SEQ ID NO: 8 or has at least 90%, 91%, 92%, 93%, 94%, 95%, 96%, 97%, 98%, 99% sequence identity to SEQ ID NO: 8, and the VL sequence is SEQ ID NO: 16 or has at least 90%, 91%, 92%, 93%, 94%, 95%, 96%, 97%, 98%, 99% sequence identity to SEQ ID NO: 16;
(2) the VH sequence is SEQ ID NO: 24 or has at least 90%, 91%, 92%, 93%, 94%, 95%, 96%, 97%, 98%, 99% sequence identity to SEQ ID NO: 24, and the VL sequence is SEQ ID NO: 32 or has at least 90%, 91%, 92%, 93%, 94%, 95%, 96%, 97%, 98%, 99% sequence identity to SEQ ID NO: 32;
(3) the VH sequence is SEQ ID NO: 40 or has at least 90%, 91%, 92%, 93%, 94%, 95%, 96%, 97%, 98%, 99% sequence identity to SEQ ID NO: 40, and the VL sequence is SEQ ID NO: 48 or has at least 90%, 91%, 92%, 93%, 94%, 95%, 96%, 97%, 98%, 99% sequence identity to SEQ ID NO: 48;
(4) the VH sequence is SEQ ID NO: 56 or has at least 90%, 91%, 92%, 93%, 94%, 95%, 96%, 97%, 98%, 99% sequence identity to SEQ ID NO: 56, and the VL sequence is SEQ ID NO: 63 or has at least 90%, 91%, 92%, 93%, 94%, 95%, 96%, 97%, 98%, 99% sequence identity to SEQ ID NO: 63;
(5) the VH sequence is SEQ ID NO: 65 or has at least 90%, 91%, 92%, 93%, 94%, 95%, 96%, 97%, 98%, 99% sequence identity to SEQ ID NO: 65, and the VL sequence is SEQ ID NO: 68 or has at least 90%, 91%, 92%, 93%, 94%, 95%, 96%, 97%, 98%, 99% sequence identity to SEQ ID NO: 68; or
(6) the VH sequence is SEQ ID NO: 75 or has at least 90%, 91%, 92%, 93%, 94%, 95%, 96%, 97%, 98%, 99% sequence identity to SEQ ID NO: 75, and the VL sequence is SEQ ID NO: 83 or has at least 90%, 91%, 92%, 93%, 94%, 95%, 96%, 97%, 98%, 99% sequence identity to SEQ ID NO: 83.

5. The isolated monoclonal antibody or antigen-binding fragment thereof according to claim 1 or 2, wherein said monoclonal antibody is a humanized antibody,
optionally, the humanized antibody comprises:
(1) the VH sequence of any one of the monoclonal antibodies in Tables B, D and E or a VH sequence least 90%, 91%, 92%, 93%, 94%, 95%, 96%, 97%, 98%, 99% identical thereto; and/or the VL sequence of any one of the monoclonal antibodies in Tables C, D and E or a VL sequence least 90%, 91%, 92%, 93%, 94%, 95%, 96%, 97%, 98%, 99% identical thereto;
(2) the VH sequence of SEQ ID NO: 96, or a VH sequence least 90%, 91%, 92%, 93%, 94%, 95%, 96%, 97%, 98%, 99% identical thereto, and the VL sequence of SEQ ID NO: 112, or a VL sequence at least 90%, 91%, 92%, 93%, 94%, 95%, 96%, 97%, 98%, 99% identical thereto;
(3) the VH sequence of SEQ ID NO: 113, or a VH sequence least 90%, 91%, 92%, 93%, 94%, 95%, 96%, 97%, 98%, 99% identical thereto, and the VL sequence of SEQ ID NO: 112, or a VL sequence at least 90%, 91%, 92%, 93%, 94%, 95%, 96%, 97%, 98%, 99% identical thereto;
(4) the VH sequence of SEQ ID NO: 96, or a VH sequence least 90%, 91%, 92%, 93%, 94%, 95%, 96%, 97%, 98%, 99% identical thereto, and the VL sequence of SEQ ID NO: 101, or a VL sequence at least 90%, 91%, 92%, 93%, 94%, 95%, 96%, 97%, 98%, 99% identical thereto;
(5) the VH sequence of SEQ ID NO: 96, or a VH sequence least 90%, 91%, 92%, 93%, 94%, 95%, 96%, 97%, 98%, 99% identical thereto, and the VL sequence of SEQ ID NO: 109, or a VL sequence at least 90%, 91%, 92%, 93%, 94%, 95%, 96%, 97%, 98%, 99% identical thereto.

6. The isolated monoclonal antibody or antigen-binding fragment thereof according to claim 1 or 2, wherein the humanized antibody comprises:
(1) the VH framework region sequences VH FR1, VH FR2, VH FR3 and VH FR4:
(i) of any one antibody in Tables B and D,
(ii) comprising amino acid sequences substantially identical (e.g., having at least about 80%, 85%, 90%, 92%, 95%, 97%, 98%, or 99% sequence identity) or identical to SEQ ID NOs: 84, 85, 86 and 87, respectively;
(iii) comprising amino acid sequences substantially identical (e.g., having at least about 80%, 85%, 90%, 92%, 95%, 97%, 98%, or 99% sequence identity) or identical to SEQ ID NOs: 89, 90, 91 and 87, respectively; or
(iv) comprising amino acid sequences substantially identical (e.g., having at least about 80%, 85%, 90%, 92%, 95%, 97%, 98%, or 99% sequence identity) or identical to SEQ ID NOs: 93, 94, 95 and 87, respectively;
(v) comprising amino acid sequences substantially identical (e.g., having at least about 80%, 85%, 90%, 92%, 95%, 97%, 98%, or 99% sequence identity) or identical to that of SEQ ID NOs: 132, 85, 133 and 87, respectively;
(vi) comprising amino acid sequences substantially identical (e.g., having at least about 80%, 85%, 90%, 92%, 95%, 97%, 98%, or 99% sequence identity) or identical to that of SEQ ID NOs: 93, 126, 127 and 87, respectively;
(vii) comprising amino acid sequences substantially identical (e.g., having at least about 80%, 85%, 90%, 92%, 95%, 97%, 98%, or 99% sequence identity) or identical to that of SEQ ID NOs: 132, 133, 134 and 87, respectively;
(viii) comprising amino acid sequences substantially identical (e.g., having at least about 80%, 85%, 90%, 92%, 95%, 97%, 98%, or 99% sequence identity) or identical to that of SEQ ID NOs: 138, 94, 139 and 87, respectively; or
(ix) comprising amino acid sequences substantially identical (e.g., having at least about 80%, 85%, 90%, 92%, 95%, 97%, 98%, or 99% sequence identity) or identical to that of SEQ ID NOs: 141, 142, 143 and 87, respectively; and/or
(2) the VL framework region sequences VL FR1, VL FR2, VL FR3 and VL FR4:
(i) of any one antibody in Tables C and D,
(ii) comprising amino acid sequences substantially identical (e.g., having at least about 80%, 85%, 90%, 92%, 95%, 97%, 98%, or 99% sequence identity) or identical to SEQ ID NOs: 97, 98, 99 and 100, respectively;
(iii) comprising amino acid sequences substantially identical (e.g., having at least about 80%, 85%, 90%, 92%, 95%, 97%, 98%, or 99% sequence identity) or identical to SEQ ID NOs: 97, 102, 99 and 100, respectively;
(iv) comprising amino acid sequences substantially identical (e.g., having at least about 80%, 85%, 90%, 92%, 95%, 97%, 98%, or 99% sequence identity) or identical to SEQ ID NOs: 103, 104, 105 and 100, respectively;
(v) comprising amino acid sequences substantially identical (e.g., having at least about 80%, 85%, 90%, 92%, 95%, 97%, 98%, or 99% sequence identity) or identical to SEQ ID NOs: 103, 107, 108 and 100, respectively;
(vi) comprising amino acid sequences substantially identical (e.g., having at least about 80%, 85%, 90%, 92%, 95%, 97%, 98%, or 99% sequence identity) or identical to that of SEQ ID NOs: 134, 135, 136 and 131, respectively;
(vii) comprising amino acid sequences substantially identical (e.g., having at least about 80%, 85%, 90%, 92%, 95%, 97%, 98%, or 99% sequence identity) or identical to that of SEQ ID NOs: 128, 129, 130 and 131, respectively;
(viii) comprising amino acid sequences substantially identical (e.g., having at least about 80%, 85%, 90%, 92%, 95%, 97%, 98%, or 99% sequence identity) or identical to that of SEQ ID NOs: 145, 146, 147 and 131, respectively;
(ix) comprising amino acid sequences substantially identical (e.g., having at least about 80%, 85%, 90%, 92%, 95%, 97%, 98%, or 99% sequence identity) or identical to that of SEQ ID NOs: 97, 98, 152 and 100, respectively; or
(x) comprising amino acid sequences substantially identical (e.g., having at least about 80%, 85%, 90%, 92%, 95%, 97%, 98%, or 99% sequence identity) or identical to that of SEQ ID NOs: 154, 102, 99 and 47, respectively.

7. The isolated monoclonal antibody or antigen-binding fragment thereof according to claim 6, wherein:
(1) the VH FR1, VH FR2, VH FR3 and VH FR4 sequences comprise
(i) SEQ ID NOs: 93, 94, 95 and 87, respectively;
(ii) SEQ ID NOs: 132, 85, 133 and 87, respectively;
(iii) SEQ ID NOs: 93, 126, 127 and 87, respectively; or
(iv) SEQ ID NOs: 132, 133, 134 and 87, respectively; and/or
(2) the VL FR1, VL FR2, VL FR3 and VL FR4 sequences comprise
(i) SEQ ID NOs: 134, 135, 136 and 131, respectively; or
(ii) SEQ ID NOs: 128, 129, 130 and 131, respectively.

8. The isolated monoclonal antibody or antigen-binding fragment thereof according to claim 7, wherein
(1) the VH sequence comprises the amino acid sequence of SEQ ID NO: 96 and the VL sequence comprises the amino acid sequence of SEQ ID NO: 112; or
(2) the VH sequence comprises the amino acid sequence of SEQ ID NO: 113 and the VL sequence comprises the amino acid sequence of SEQ ID NO: 112.

9. The isolated monoclonal antibody or antigen-binding fragment thereof according to claim 1 or 2, wherein the VH sequence comprises the amino acid sequence of SEQ ID NO: 56 and VL sequence comprises the amino acid sequence of SEQ ID NO: 111.

10. The isolated monoclonal antibody or antigen-binding fragment thereof according to any one of claims 1-9, comprising a modified Fc region to enhance ADCC.

11. The isolated monoclonal antibody or antigen-binding fragment thereof according to claim 10, wherein the modified Fc region comprises:
(1) F243L/R292P/Y300L/V305I/P396L mutations to enhance FcγRIIIa binding;
(2) S239D/I332E mutations to enhance FcγRIIIa binding;
(3) S239D/I332E/A330L mutations to simultaneously enhance FcγRIIIa binding and decrease FcγRIIIb binding;
(4) S298A/E333A/K334A mutations to enhance FcγRIIIa binding; and/or
(5) afucosylated N297 at Fc region to enhance FcγRIIIa binding.

12. The isolated monoclonal antibody or antigen-binding fragment thereof according to any one of claims 1-9, wherein said antigen-binding fragment thereof is an Fab, Fab′, F(ab′)₂, F_{d}, single chain Fv or scFv, disulfide linked Fᵥ, V-NAR domain, IgNar, intrabody, IgGΔCH₂, minibody, F(ab′)₃, tetrabody, triabody, diabody, single-domain antibody, DVD-Ig, Fcab, mAb₂, (scFv)₂, or scFv-Fc.

13. The isolated monoclonal antibody or antigen-binding fragment thereof according to any one of claims 1-12, having a low (e.g., 1-5 or 1-2) pM range EC50 value for ADCC activity.

14. The isolated monoclonal antibody or antigen-binding fragment thereof according to any one of claims 1-13, having ADCC activity against primary B cells expressing surface hCXCR5, and/or primary T cells expressing surface hCXCR5.

15. The isolated monoclonal antibody or antigen-binding fragment thereof according to any one of claims 1-14, which does not (or at most minimally) internalize the hCXCR5 surface antigen.

16. The isolated monoclonal antibody or antigen-binding fragment thereof according to any one of claims 1-15, which inhibits cAMP signaling (e.g., EC50 less than 1 nM).

17. The isolated monoclonal antibody or antigen-binding fragment thereof according to any one of claims 1-16, which inhibits chemotaxis (e.g., with ˜100% inhibition at about 0.1-0.5 nM, or about 0.1 nM).

18. The isolated monoclonal antibody or antigen-binding fragment thereof according to any one of claims 1-17, which inhibits hCXCL13-induced B cell migration.

19. The isolated monoclonal antibody or antigen-binding fragment thereof according to any one of claims 1-18, which does not substantially cross-react with hCXCR3.

20. The isolated monoclonal antibody or antigen-binding fragment thereof according to any one of claims 1-19, which binds to hCXCR5 expressed on adherent cell lines (such as DX002) and/or suspension cell lines (such as M300-19).

21. The isolated monoclonal antibody or antigen-binding fragment thereof according to any one of claims 1-20, which does not or minimally cross-reacts with cynomolgus monkey or mouse orthologs of hCXCR5.

22. The isolated monoclonal antibody or antigen-binding fragment thereof according to any one of claims 1-21, which reduces the percentage of memory B cell population in a subject.

23. The isolated monoclonal antibody or antigen-binding fragment thereof of any one of claims 1-22, which binds hCXCR5 with a K_{d} of less than about 25 nM, 20 nM, 15 nM, 10 nM, 5 nM, 2 nM, or 1 nM or less.

24. An isolated monoclonal antibody or an antigen-binding fragment thereof, which competes with the isolated monoclonal antibody or antigen-binding fragment thereof of any one of claims 1-23 for binding to the same epitope.

25. A method of treating Sjögren syndrome (SS) in a subject in need thereof, the method comprising administering a therapeutically effective amount of an antibody of any one of claims 1-24 to the subject.

26. The method of claim 25, which alleviates at least one symptom of SS.

27. A method of treating a disease or indication with ectopic germinal centers (GCs), including autoimmune disease or disorder, in a subject in need thereof, the method comprising administering a therapeutically effective amount of an antibody of any one of claims 1-24 to the subject.

28. The method of claim 27, wherein the disease or indication is Rheumatoid Arthritis (RA), systemic lupus erythematosus (SLE), Celiac disease, Crohn's disease, ulcerative colitis, type I diabetes, multiple sclerosis (MS), Sarcoidosis, Psoriasis, Myasthenia gravis, Hashimoto's thyroiditis, Grave's disease, artherosclerosis, conjunctivitis, gastritis, hepatitis, or dermatitis.

29. A method of treating lymphoma or leukemia in a subject in need thereof, the method comprising administering a therapeutically effective amount of an antibody of any one of claims 1-24 to the subject.

30. The method of claim 29, wherein the lymphoma or leukemia is B cell lymphoma.

31. The method of claim 30, wherein the B cell lymphoma is CLL (B-cell Chronic Lymphocytic Leukemia).

32. The method of claim 30, wherein the lymphoma or leukemia is non-Hodgkin's lymphoma, such as Burkitt's lymphoma.

33. A method of treating solid cancer in a subject in need thereof, the method comprising administering a therapeutically effective amount of an antibody of any one of claims 1-24 to the subject, wherein the solid cancer is gastric cancer, breast cancer, intestinal cancer, lung cancer, or prostate cancer.

34. The method of any one of claims 29-33, further comprising administering to the patient a chemotherapeutic agent, an anti-angiogenesis agent, a growth inhibitory agent, an immune-oncology agent, and/or an anti-neoplastic composition.

35. A polynucleotide encoding the heavy chain or the light chain or the antigen-binding portion thereof of any one of claims 1-24.

36. The polynucleotide of claim 35, which is codon optimized for expression in a human cell.

37. A vector comprising the polynucleotide of claim 35 or 36.

38. The vector of claim 37, which is an expression vector (e.g., a mammalian expression vector, a yeast expression vector, an insect expression vector, or a bacterial expression vector).


SEQUENCE LISTING (187 sequences)

SEQ ID NO: 1 - protein, 8 aa, Artificial Sequence
  Description of Artificial Sequence: Synthetic peptide
GFTFSNYW

SEQ ID NO: 2 - protein, 10 aa, Artificial Sequence
  Description of Artificial Sequence: Synthetic peptide
IRLKSDNYAT

SEQ ID NO: 3 - protein, 7 aa, Artificial Sequence
  Description of Artificial Sequence: Synthetic peptide
TPPNFDY

SEQ ID NO: 4 - protein, 25 aa, Artificial Sequence
  Description of Artificial Sequence: Synthetic peptide
EVKLEESGGGLVQPGGSMKLSCVAS

SEQ ID NO: 5 - protein, 17 aa, Artificial Sequence
  Description of Artificial Sequence: Synthetic peptide
MNWVRQSPEKGLEWVAQ

SEQ ID NO: 6 - protein, 38 aa, Artificial Sequence
  Description of Artificial Sequence: Synthetic polypeptide
HYTESVKGRFTISRDDSKSSVYLQMNNLRAEDTGIYYC

SEQ ID NO: 7 - protein, 11 aa, Artificial Sequence
  Description of Artificial Sequence: Synthetic peptide
WGQGTTLTVSS

SEQ ID NO: 8 - protein, 116 aa, Artificial Sequence
  Description of Artificial Sequence: Synthetic polypeptide
EVKLEESGGGLVQPGGSMKLSCVASGFTFSNYWMNWVRQSPEKGLEWVAQIRLKSDNYATHYTESVKGRFTISRDDSKSSVYLQMNNLRAEDTGIYYCTPPNFDYWGQGTTLTVSS

SEQ ID NO: 9 - protein, 11 aa, Artificial Sequence
  Description of Artificial Sequence: Synthetic peptide
TSLLHRSGKHK

SEQ ID NO: 10 - protein, 3 aa, Artificial Sequence
  Description of Artificial Sequence: Synthetic peptide
YVS

SEQ ID NO: 11 - protein, 9 aa, Artificial Sequence
  Description of Artificial Sequence: Synthetic peptide
MQSLEFPLT

SEQ ID NO: 12 - protein, 26 aa, Artificial Sequence
  Description of Artificial Sequence: Synthetic peptide
DIVMTQAAPSVTVTPGESVTISCRST

SEQ ID NO: 13 - protein, 17 aa, Artificial Sequence
  Description of Artificial Sequence: Synthetic peptide
FYWFLQRPGQSPQLLIY

SEQ ID NO: 14 - protein, 36 aa, Artificial Sequence
  Description of Artificial Sequence: Synthetic polypeptide
NLASGVPDRFSGSGSGTDFTLRISRVEAEDFGVYYC

SEQ ID NO: 15 - protein, 10 aa, Artificial Sequence
  Description of Artificial Sequence: Synthetic peptide
FGTGTKLEIK

SEQ ID NO: 16 - protein, 112 aa, Artificial Sequence
  Description of Artificial Sequence: Synthetic polypeptide
DIVMTQAAPSVTVTPGESVTISCRSTTSLLHRSGKHKFYWFLQRPGQSPQLLIYYVSNLASGVPDRFSGSGSGTDFTLRISRVEAEDFGVYYCMQSLEFPLTFGTGTKLEIK

SEQ ID NO: 17 - protein, 8 aa, Artificial Sequence
  Description of Artificial Sequence: Synthetic peptide
GFTFNTNA

SEQ ID NO: 18 - protein, 10 aa, Artificial Sequence
  Description of Artificial Sequence: Synthetic peptide
IRSKSNNYAT

SEQ ID NO: 19 - protein, 8 aa, Artificial Sequence
  Description of Artificial Sequence: Synthetic peptide
VSWDPFAY

SEQ ID NO: 20 - protein, 25 aa, Artificial Sequence
  Description of Artificial Sequence: Synthetic peptide
EVQLVETGGGLVQPKGSLKLSCAAS

SEQ ID NO: 21 - protein, 17 aa, Artificial Sequence
  Description of Artificial Sequence: Synthetic peptide
MNWVRQAPGKGLEWVAR

SEQ ID NO: 22 - protein, 38 aa, Artificial Sequence
  Description of Artificial Sequence: Synthetic polypeptide
HYVDSVKDRFTISRDDSQSMLYLQMNNLKTEDTAMYYC

SEQ ID NO: 23 - protein, 11 aa, Artificial Sequence
  Description of Artificial Sequence: Synthetic peptide
WGQGTLVTVSA

SEQ ID NO: 24 - protein, 117 aa, Artificial Sequence
  Description of Artificial Sequence: Synthetic polypeptide
EVQLVETGGGLVQPKGSLKLSCAASGFTFNTNAMNWVRQAPGKGLEWVARIRSKSNNYATHYVDSVKDRFTISRDDSQSMLYLQMNNLKTEDTAMYYCVSWDPFAYWGQGTLVTVSA

SEQ ID NO: 25 - protein, 12 aa, Artificial Sequence
  Description of Artificial Sequence: Synthetic peptide
QSVLYSSNQKNY

SEQ ID NO: 26 - protein, 3 aa, Artificial Sequence
  Description of Artificial Sequence: Synthetic peptide
WAS

SEQ ID NO: 27 - protein, 8 aa, Artificial Sequence
  Description of Artificial Sequence: Synthetic peptide
HQYLSSWT

SEQ ID NO: 28 - protein, 26 aa, Artificial Sequence
  Description of Artificial Sequence: Synthetic peptide
NIMMTQSPSSLAVSAGEKVTMSCKSS

SEQ ID NO: 29 - protein, 17 aa, Artificial Sequence
  Description of Artificial Sequence: Synthetic peptide
LAWYQQKPGQSPKLLIY

SEQ ID NO: 30 - protein, 36 aa, Artificial Sequence
  Description of Artificial Sequence: Synthetic polypeptide
TRASGVPDRFTGSGSGTDFTLTISSVQTEDLAVYYC

SEQ ID NO: 31 - protein, 10 aa, Artificial Sequence
  Description of Artificial Sequence: Synthetic peptide
FGGGAKLEIK

SEQ ID NO: 32 - protein, 112 aa, Artificial Sequence
  Description of Artificial Sequence: Synthetic polypeptide
NIMMTQSPSSLAVSAGEKVTMSCKSSQSVLYSSNQKNYLAWYQQKPGQSPKLLIYWASTRASGVPDRFTGSGSGTDFTLTISSVQTEDLAVYYCHQYLSSWTFGGGAKLEIK

SEQ ID NO: 33 - protein, 8 aa, Artificial Sequence
  Description of Artificial Sequence: Synthetic peptide
GFSLTSYG

SEQ ID NO: 34 - protein, 7 aa, Artificial Sequence
  Description of Artificial Sequence: Synthetic peptide
TWSGGRT

SEQ ID NO: 35 - protein, 6 aa, Artificial Sequence
  Description of Artificial Sequence: Synthetic peptide
ARGGNY

SEQ ID NO: 36 - protein, 25 aa, Artificial Sequence
  Description of Artificial Sequence: Synthetic peptide
QVQLKQSGPGLVQPSQSLSITCTVS

SEQ ID NO: 37 - protein, 17 aa, Artificial Sequence
  Description of Artificial Sequence: Synthetic peptide
VHWVRQSPGKGLEWLGV

SEQ ID NO: 38 - protein, 38 aa, Artificial Sequence
  Description of Artificial Sequence: Synthetic polypeptide
DYNAAFISRLSINKDNSKSQVFFKMNSLQVNDTAIYYC

SEQ ID NO: 39 - protein, 11 aa, Artificial Sequence
  Description of Artificial Sequence: Synthetic peptide
WGQGTSVTVSS

SEQ ID NO: 40 - protein, 112 aa, Artificial Sequence
  Description of Artificial Sequence: Synthetic polypeptide
QVQLKQSGPGLVQPSQSLSITCTVSGFSLTSYGVHWVRQSPGKGLEWLGVTWSGGRTDYNAAFISRLSINKDNSKSQVFFKMNSLQVNDTAIYYCARGGNYWGQGTSVTVSS

SEQ ID NO: 41 - protein, 10 aa, Artificial Sequence
  Description of Artificial Sequence: Synthetic peptide
ESVDNYGISF

SEQ ID NO: 42 - protein, 3 aa, Artificial Sequence
  Description of Artificial Sequence: Synthetic peptide
AAS

SEQ ID NO: 43 - protein, 9 aa, Artificial Sequence
  Description of Artificial Sequence: Synthetic peptide
QQSKEVPWT

SEQ ID NO: 44 - protein, 26 aa, Artificial Sequence
  Description of Artificial Sequence: Synthetic peptide
DIVLTQSPASLTVSLGQRATISCRAS

SEQ ID NO: 45 - protein, 17 aa, Artificial Sequence
  Description of Artificial Sequence: Synthetic peptide
MNWFQQKPGQPPKLLIY

SEQ ID NO: 46 - protein, 36 aa, Artificial Sequence
  Description of Artificial Sequence: Synthetic polypeptide
NQGSGVPARFSGSGSGTDFSLNIHPMEEDDTAMYFC

SEQ ID NO: 47 - protein, 10 aa, Artificial Sequence
  Description of Artificial Sequence: Synthetic peptide
FGGGTKLEIK

SEQ ID NO: 48 - protein, 222 aa, Artificial Sequence
  Description of Artificial Sequence: Synthetic polypeptide
DIVLTQSPASLTVSLGQRATISCRASESVDNYGISFMNWFQQKPGQPPKLLIYAASNQGSGVPARFSGSGSGTDQVQLKESGPGLVAPSQSLSITCTVSGFSLTSYGVSWVRQPPGKGLEWLGVIWGDGSTNYHSTLISRLSISKDNSKSQVFLKLNSLQTDDTATYYCARVAYWGQGTLVTVSAFSLNIHPMEEDDTAMYFCQQSKEVPWTFGGGTKLEIK

SEQ ID NO: 49 - protein, 7 aa, Artificial Sequence
  Description of Artificial Sequence: Synthetic peptide
IWGDGST

SEQ ID NO: 50 - protein, 26 aa, Artificial Sequence [sequence omitted]

SEQ ID NO: 51 - protein, 5 aa, Artificial Sequence
  Description of Artificial Sequence: Synthetic peptide
ARVVY

SEQ ID NO: 52 - protein, 25 aa, Artificial Sequence
  Description of Artificial Sequence: Synthetic peptide
QVQLKESGPGLVAPSQSLSITCTVS

SEQ ID NO: 53 - protein, 17 aa, Artificial Sequence
  Description of Artificial Sequence: Synthetic peptide
VSWVRQPPGKGLEWLGV

SEQ ID NO: 54 - protein, 38 aa, Artificial Sequence
  Description of Artificial Sequence: Synthetic polypeptide
NYHSGLISRLSISKDNSKSQVFLKLNSLQSDDTATYYC

SEQ ID NO: 55 - protein, 11 aa, Artificial Sequence
  Description of Artificial Sequence: Synthetic peptide
WGQGTLVTVSA

SEQ ID NO: 56 - protein, 111 aa, Artificial Sequence
  Description of Artificial Sequence: Synthetic polypeptide
QVQLKESGPGLVAPSQSLSITCTVSGFSLTSYGVSWVRQPPGKGLEWLGVIWGDGSTNYHSGLISRLSISKDNSKSQVFLKLNSLQSDDTATYYCARVVYWGQGTLVTVSA

SEQ ID NO: 57 - protein, 11 aa, Artificial Sequence
  Description of Artificial Sequence: Synthetic peptide
KSLLHSNGKTY

SEQ ID NO: 58 - protein, 3 aa, Artificial Sequence
  Description of Artificial Sequence: Synthetic peptide
RMS

SEQ ID NO: 59 - protein, 9 aa, Artificial Sequence
  Description of Artificial Sequence: Synthetic peptide
MQHLEYPYT

SEQ ID NO: 60 - protein, 26 aa, Artificial Sequence
  Description of Artificial Sequence: Synthetic peptide
DIVMTQAAPSVPVTPGESISISCRSS

SEQ ID NO: 61 - protein, 17 aa, Artificial Sequence
  Description of Artificial Sequence: Synthetic peptide
LYWFLQRPGQSPQLLLY

SEQ ID NO: 62 - protein, 36 aa, Artificial Sequence
  Description of Artificial Sequence: Synthetic polypeptide
NLASGVPDRFSGSGSGTAFTLRISRVEAEDVGVYYC

SEQ ID NO: 63 - protein, 112 aa, Artificial Sequence
  Description of Artificial Sequence: Synthetic polypeptide
DIVMTQAAPSVPVTPGESISISCRSSKSLLHSNGKTYLYWFLQRPGQSPQLLLYRMSNLASGVPDRFSGSGSGTAFTLRISRVEAEDVGVYYCMQHLEYPYTFGGGTKLEIK

SEQ ID NO: 64 - protein, 38 aa, Artificial Sequence
  Description of Artificial Sequence: Synthetic polypeptide
NYHSTLISRLSISKDNSKSQVFLKLNSLQTDDTATYYC

SEQ ID NO: 65 - protein, 5 aa, Artificial Sequence
  Description of Artificial Sequence: Synthetic peptide
ARVAY

SEQ ID NO: 66 - protein, 26 aa, Artificial Sequence
  Description of Artificial Sequence: Synthetic peptide
DIVMTQAAPSIPVTPGESVSISCRSS

SEQ ID NO: 67 - protein, 17 aa, Artificial Sequence
  Description of Artificial Sequence: Synthetic peptide
LYWFLQRPGQSPQLLIY

SEQ ID NO: 68 - protein, 112 aa, Artificial Sequence
  Description of Artificial Sequence: Synthetic polypeptide
DIVMTQAAPSIPVTPGESVSISCRSSKSLLHSNGKTYLYWFLQRPGQSPQLLIYRMSNLASGVPDRFSGSGSGTAFTLRISRVEAEDVGVYYCMQHLEYPYTFGGGTKLEIK

SEQ ID NO: 69 - protein, 8 aa, Artificial Sequence
  Description of Artificial Sequence: Synthetic peptide
GYTFTDYT

SEQ ID NO: 70 - protein, 8 aa, Artificial Sequence
  Description of Artificial Sequence: Synthetic peptide
IYPGSGNI

SEQ ID NO: 71 - protein, 10 aa, Artificial Sequence
  Description of Artificial Sequence: Synthetic peptide
ARGLRREFAY

SEQ ID NO: 72 - protein, 25 aa, Artificial Sequence
  Description of Artificial Sequence: Synthetic peptide
QIQLQQSGPELVEPGASVKLSCKAS

SEQ ID NO: 73 - protein, 17 aa, Artificial Sequence
  Description of Artificial Sequence: Synthetic peptide
IHWVKQSPGQGLEWIGW

SEQ ID NO: 74 - protein, 38 aa, Artificial Sequence
  Description of Artificial Sequence: Synthetic polypeptide
KYNDKFKGKATMTADKSSSTAYMQLSSLTSEDSAVYFC

SEQ ID NO: 75 - protein, 117 aa, Artificial Sequence
  Description of Artificial Sequence: Synthetic polypeptide
QIQLQQSGPELVEPGASVKLSCKASGYTFTDYTIHWVKQSPGQGLEWIGWIYPGSGNIKYNDKFKGKATMTADKSSSTAYMQLSSLTSEDSAVYFCARGLRREFAYWGQGTLVTVSA

SEQ ID NO: 76 - protein, 11 aa, Artificial Sequence
  Description of Artificial Sequence: Synthetic peptide
KSLLHSNGITY

SEQ ID NO: 77 - protein, 3 aa, Artificial Sequence
  Description of Artificial Sequence: Synthetic peptide
QMS

SEQ ID NO: 78 - protein, 9 aa, Artificial Sequence
  Description of Artificial Sequence: Synthetic peptide
AQSLELPLT

SEQ ID NO: 79 - protein, 26 aa, Artificial Sequence
  Description of Artificial Sequence: Synthetic peptide
DIVMTQAAFSNPVTLGTSASISCRSS

SEQ ID NO: 80 - protein, 17 aa, Artificial Sequence
  Description of Artificial Sequence: Synthetic peptide
LYWYLQKPGQSPQLLIY

SEQ ID NO: 81 - protein, 36 aa, Artificial Sequence
  Description of Artificial Sequence: Synthetic polypeptide
NLASGVPDRFSSSGSGTDFTLRISRVEAEDVGVYYC

SEQ ID NO: 82 - protein, 10 aa, Artificial Sequence
  Description of Artificial Sequence: Synthetic peptide
FGAGTKLELK

SEQ ID NO: 83 - protein, 112 aa, Artificial Sequence
  Description of Artificial Sequence: Synthetic polypeptide
DIVMTQAAFSNPVTLGTSASISCRSSKSLLHSNGITYLYWYLQKPGQSPQLLIYQMSNLASGVPDRFSSSGSGTDFTLRISRVEAEDVGVYYCAQSLELPLTFGAGTKLELK

SEQ ID NO: 84 - protein, 25 aa, Artificial Sequence [sequence omitted]

SEQ ID NO: 85 - protein, 17 aa, Artificial Sequence
  Description of Artificial Sequence: Synthetic peptide
VSWVRQAPGQGLEWMGV

SEQ ID NO: 86 - protein, 38 aa, Artificial Sequence
  Description of Artificial Sequence: Synthetic polypeptide
NYAQKFQGRVTITKDESTSTAYMELSSLRSEDTAVYYC

SEQ ID NO: 87 - protein, 11 aa, Artificial Sequence
  Description of Artificial Sequence: Synthetic peptide
WGQGTLVTVSS

SEQ ID NO: 88 - protein, 111 aa, Artificial Sequence
  Description of Artificial Sequence: Synthetic polypeptide
QVQLVQSGAEVKKPGSSVKVSCKASGFSLTSYGVSWVRQAPGQGLEWMGVIWGDGSTNYAQKFQGRVTITKDESTSTAYMELSSLRSEDTAVYYCARVVYWGQGTLVTVSS

SEQ ID NO: 89 - protein, 25 aa, Artificial Sequence [sequence omitted]

SEQ ID NO: 90 - protein, 17 aa, Artificial Sequence
  Description of Artificial Sequence: Synthetic peptide
VSWVRQAPGKGLEWVGV

SEQ ID NO: 91 - protein, 38 aa, Artificial Sequence
  Description of Artificial Sequence: Synthetic polypeptide
NYAASVKGRFTISRDDSKNSVYLQMNSLKTEDTAVYYC

SEQ ID NO: 92 - protein, 111 aa, Artificial Sequence
  Description of Artificial Sequence: Synthetic polypeptide
EVQLVESGGGLVQPGGSLRLSCAASGFSLTSYGVSWVRQAPGKGLEWVGVIWGDGSTNYAASVKGRFTISRDDSKNSVYLQMNSLKTEDTAVYYCARVVYWGQGTLVTVSS

SEQ ID NO: 93 - protein, 25 aa, Artificial Sequence
  Description of Artificial Sequence: Synthetic peptide
QVQLQESGPGLVKPSETLSLTCTVS

SEQ ID NO: 94 - protein, 17 aa, Artificial Sequence
  Description of Artificial Sequence: Synthetic peptide
VSWVRQPPGKGLEWIGV

SEQ ID NO: 95 - protein, 38 aa, Artificial Sequence
  Description of Artificial Sequence: Synthetic polypeptide
NYNPSLKSRVTISKDTSKNQVSLKLSSVTAADTAVYYC

SEQ ID NO: 96 - protein, 111 aa, Artificial Sequence
  Description of Artificial Sequence: Synthetic polypeptide
QVQLQESGPGLVKPSETLSLTCTVSGFSLTSYGVSWVRQPPGKGLEWIGVIWGDGSTNYNPSLKSRVTISKDTSKNQVSLKLSSVTAADTAVYYCARVVYWGQGTLVTVSS

SEQ ID NO: 97 - protein, 26 aa, Artificial Sequence
  Description of Artificial Sequence: Synthetic peptide
DIVMTQSPLSLPVTPGEPASISCRSS

SEQ ID NO: 98 - protein, 17 aa, Artificial Sequence
  Description of Artificial Sequence: Synthetic peptide
LYWFLQKPGQSPQLLIY

SEQ ID NO: 99 - protein, 36 aa, Artificial Sequence
  Description of Artificial Sequence: Synthetic polypeptide
NLASGVPDRFSGSGSGTDFTLKISRVEAEDVGVYYC

SEQ ID NO: 100 - protein, 10 aa, Artificial Sequence
  Description of Artificial Sequence: Synthetic peptide
FGGGTKVEIK

SEQ ID NO: 101 - protein, 112 aa, Artificial Sequence
  Description of Artificial Sequence: Synthetic polypeptide
DIVMTQSPLSLPVTPGEPASISCRSSKSLLHSNGKTYLYWFLQKPGQSPQLLIYRMSNLASGVPDRFSGSGSGTDFTLKISRVEAEDVGVYYCMQHLEYPYTFGGGTKVEIK

SEQ ID NO: 102 - protein, 17 aa, Artificial Sequence
  Description of Artificial Sequence: Synthetic peptide
LYWFLQKPGQSPQLLLY

SEQ ID NO: 103 - protein, 26 aa, Artificial Sequence [sequence omitted]

SEQ ID NO: 104 - protein, 17 aa, Artificial Sequence
  Description of Artificial Sequence: Synthetic peptide
LYWFQQKPGKAPKLLLY

SEQ ID NO: 105 - protein, 36 aa, Artificial Sequence
  Description of Artificial Sequence: Synthetic polypeptide
NLASGVPSRFSGSGSGTDFTLTISSLQPEDVATYYC

SEQ ID NO: 106 - protein, 112 aa, Artificial Sequence
  Description of Artificial Sequence: Synthetic polypeptide
DIQMTQSPSSLSASVGDRVTITCRSSKSLLHSNGKTYLYWFQQKPGKAPKLLLYRMSNLASGVPSRFSGSGSGTDFTLTISSLQPEDVATYYCMQHLEYPYTFGGGTKVEIK

SEQ ID NO: 107 - protein, 17 aa, Artificial Sequence
  Description of Artificial Sequence: Synthetic peptide
LYWFQQKPGKAPKLLIY

SEQ ID NO: 108 - protein, 36 aa, Artificial Sequence
  Description of Artificial Sequence: Synthetic polypeptide
NLASGVPSRFSGSGSGTDFTLTISSLQPEDFATYYC

SEQ ID NO: 109 - protein, 112 aa, Artificial Sequence
  Description of Artificial Sequence: Synthetic polypeptide
DIQMTQSPSSLSASVGDRVTITCRSSKSLLHSNGKTYLYWFQQKPGKAPKLLIYRMSNLASGVPSRFSGSGSGTDFTLTISSLQPEDFATYYCMQHLEYPYTFGGGTKVEIK

SEQ ID NO: 110 - protein, 38 aa, Artificial Sequence
  Description of Artificial Sequence: Synthetic polypeptide
NYAQKLQGRVTMTKDTSTSTAYMELRSLRSDDTAVYYC

SEQ ID NO: 111 - skipped

SEQ ID NO: 112 - protein, 112 aa, Artificial Sequence
  Description of Artificial Sequence: Synthetic polypeptide
EIVMTQSPATLSVSPGERATLSCRSSKSLLHSNGKTYLYWFQQKPGQAPRLLIYRMSNLASGIPARFSGSGSGTEFTLTISSVQSEDVAVYYCMQHLEYPYTFGQGTKLEIK

SEQ ID NO: 113 - protein, 111 aa, Artificial Sequence
  Description of Artificial Sequence: Synthetic polypeptide
QVQLVQSGAEVKKPGASVKVSCKASGFSLTSYGVSWVRQAPGQGLEWMGVIWGDGSTNYAQKLQGRVTMTKDTSTSTAYMELRSLRSDDTAVYYCARVVYWGQGTLVTVSS

SEQ ID NO: 114 - protein, 10 aa, Artificial Sequence
  Description of Artificial Sequence: Synthetic peptide
GFSLTSYGVS

SEQ ID NO: 115 - protein, 9 aa, Artificial Sequence
  Description of Artificial Sequence: Synthetic peptide
VIWGDGSTN

SEQ ID NO: 116 - protein, 3 aa, Artificial Sequence
  Description of Artificial Sequence: Synthetic peptide
VVY

SEQ ID NO: 117 - protein, 14 aa, Artificial Sequence
  Description of Artificial Sequence: Synthetic peptide
WVRQPPGKGLEWLG

SEQ ID NO: 118 - protein, 39 aa, Artificial Sequence
  Description of Artificial Sequence: Synthetic polypeptide
YHSGLISRLSISKDNSKSQVFLKLNSLQSDDTATYYCAR

SEQ ID NO: 119 - protein, 11 aa, Artificial Sequence
  Description of Artificial Sequence: Synthetic peptide
WGQGTLVTVSA

SEQ ID NO: 120 - protein, 16 aa, Artificial Sequence
  Description of Artificial Sequence: Synthetic peptide
RSSKSLLHSNGKTYLY

SEQ ID NO: 121 - protein, 7 aa, Artificial Sequence
  Description of Artificial Sequence: Synthetic peptide
RMSNLAS

SEQ ID NO: 122 - protein, 9 aa, Artificial Sequence
  Description of Artificial Sequence: Synthetic peptide
MQHLEYPYT

SEQ ID NO: 123 - protein, 23 aa, Artificial Sequence
  Description of Artificial Sequence: Synthetic peptide
DIVMTQAAPSVPVTPGESISISC

SEQ ID NO: 124 - protein, 14 aa, Artificial Sequence
  Description of Artificial Sequence: Synthetic peptide
WFLQRPGQSPQLLL

SEQ ID NO: 125 - protein, 32 aa, Artificial Sequence
  Description of Artificial Sequence: Synthetic polypeptide
GVPDRFSGSGSGTAFTLRISRVEAEDVGVYYC

SEQ ID NO: 126 - protein, 14 aa, Artificial Sequence
  Description of Artificial Sequence: Synthetic peptide
WVRQPPGKGLEWIG

SEQ ID NO: 127 - protein, 39 aa, Artificial Sequence
  Description of Artificial Sequence: Synthetic polypeptide
YNPSLKSRVTISKDTSKNQVSLKLSSVTAADTAVYYCAR

SEQ ID NO: 128 - protein, 23 aa, Artificial Sequence
  Description of Artificial Sequence: Synthetic peptide
EIVMTQSPATLSVSPGERATLSC

SEQ ID NO: 129 - protein, 15 aa, Artificial Sequence
  Description of Artificial Sequence: Synthetic peptide
WFQQKPGQAPRLLIY

SEQ ID NO: 130 - protein, 32 aa, Artificial Sequence
  Description of Artificial Sequence: Synthetic polypeptide
GIPARFSGSGSGTEFTLTISSVQSEDVAVYYC

SEQ ID NO: 131 - protein, 10 aa, Artificial Sequence
  Description of Artificial Sequence: Synthetic peptide
FGQGTKLEIK

SEQ ID NO: 132 - protein, 25 aa, Artificial Sequence [sequence omitted]

SEQ ID NO: 133 - protein, 14 aa, Artificial Sequence
  Description of Artificial Sequence: Synthetic peptide
WVRQAPGQGLEWMG

SEQ ID NO: 134 - protein, 39 aa, Artificial Sequence
  Description of Artificial Sequence: Synthetic polypeptide
YAQKLQGRVTMTKDTSTSTAYMELRSLRSDDTAVYYCAR

SEQ ID NO: 135 - protein, 17 aa, Artificial Sequence
  Description of Artificial Sequence: Synthetic peptide
LYWFQQKPGQAPRLLIY

SEQ ID NO: 136 - protein, 36 aa, Artificial Sequence
  Description of Artificial Sequence: Synthetic polypeptide
NLASGIPARFSGSGSGTEFTLTISSVQSEDVAVYYC

SEQ ID NO: 137 - protein, 112 aa, Artificial Sequence
  Description of Artificial Sequence: Synthetic polypeptide
DIVMTQSPLSLPVTPGEPASISCRSSKSLLHSNGKTYLYWFLQKPGQSPQLLLYRMSNLASGVPDRFSGSGSGTDFTLKISRVEAEDVGVYYCMQHLEYPYTFGGGTKVEIK

SEQ ID NO: 138 - protein, 25 aa, Artificial Sequence
  Description of Artificial Sequence: Synthetic peptide
QVQLQESGPGLVKPSGTLSLTCAVS

SEQ ID NO: 139 - protein, 38 aa, Artificial Sequence
  Description of Artificial Sequence: Synthetic polypeptide
NYNPSLKSRLTISKDKSKNQVSLKLSSVTAADTAVYYC

SEQ ID NO: 140 - protein, 111 aa, Artificial Sequence
  Description of Artificial Sequence: Synthetic polypeptide
QVQLQESGPGLVKPSGTLSLTCAVSGFSLTSYGVSWVRQPPGKGLEWIGVIWGDGSTNYNPSLKSRLTISKDKSKNQVSLKLSSVTAADTAVYYCARVVYWGQGTLVTVSS

SEQ ID NO: 141 - protein, 25 aa, Artificial Sequence
  Description of Artificial Sequence: Synthetic peptide
QVQLVESGGGVVQPGRSLRLSCAVS

SEQ ID NO: 142 - protein, 17 aa, Artificial Sequence
  Description of Artificial Sequence: Synthetic peptide
VSWVRQAPGKGLEWLGV

SEQ ID NO: 143 - protein, 38 aa, Artificial Sequence
  Description of Artificial Sequence: Synthetic polypeptide
NYHSGLISRLTISKDNSKNTVYLQMNSLRAEDTAVYYC

SEQ ID NO: 144 - protein, 111 aa, Artificial Sequence
  Description of Artificial Sequence: Synthetic polypeptide
QVQLVESGGGVVQPGRSLRLSCAVSGFSLTSYGVSWVRQAPGKGLEWLGVIWGDGSTNYHSGLISRLTISKDNSKNTVYLQMNSLRAEDTAVYYCARVVYWGQGTLVTVSS

SEQ ID NO: 145 - protein, 26 aa, Artificial Sequence [sequence omitted]

SEQ ID NO: 146 - protein, 17 aa, Artificial Sequence
  Description of Artificial Sequence: Synthetic peptide
LYWFQQKPGQSPKLLLY

SEQ ID NO: 147 - protein, 36 aa, Artificial Sequence
  Description of Artificial Sequence: Synthetic polypeptide
NLASGVPDRFSGSGSGTDFTLTISSLQAEDVAVYYC

SEQ ID NO: 148 - protein, 112 aa, Artificial Sequence
  Description of Artificial Sequence: Synthetic polypeptide
DIVMTQSPDSLAVSLGERATINCRSSKSLLHSNGKTYLYWFQQKPGQSPKLLLYRMSNLASGVPDRFSGSGSGTDFTLTISSLQAEDVAVYYCMQHLEYPYTFGQGTKLEIK

SEQ ID NO: 149 - protein, 11 aa, Artificial Sequence
  Description of Artificial Sequence: Synthetic peptide
QSLLHSNGYNY

SEQ ID NO: 150 - protein, 3 aa, Artificial Sequence
  Description of Artificial Sequence: Synthetic peptide
RGS

SEQ ID NO: 151 - protein, 9 aa, Artificial Sequence
  Description of Artificial Sequence: Synthetic peptide
MQHLQYPYT

SEQ ID NO: 152 - protein, 36 aa, Artificial Sequence
  Description of Artificial Sequence: Synthetic polypeptide
NRASGVPDRFSGSGSGTDFTLKISRVEAEDVGVYYC

SEQ ID NO: 153 - protein, 112 aa, Artificial Sequence
  Description of Artificial Sequence: Synthetic polypeptide
DIVMTQSPLSLPVTPGEPASISCRSSQSLLHSNGYNYLYWFLQKPGQSPQLLIYRGSNRASGVPDRFSGSGSGTDFTLKISRVEAEDVGVYYCMQHLQYPYTFGGGTKVEIK

SEQ ID NO: 154 - protein, 26 aa, Artificial Sequence
  Description of Artificial Sequence: Synthetic peptide
DIVMTQSPLSLPVTPGEPISLSCRSS

SEQ ID NO: 155 - protein, 112 aa, Artificial Sequence
  Description of Artificial Sequence: Synthetic polypeptide
DIVMTQSPLSLPVTPGEPISLSCRSSKSLLHSNGKTYLYWFLQKPGQSPQLLLYRMSNLASGVPDRFSGSGSGTDFTLKISRVEAEDVGVYYCMQHLEYPYTFGGGTKLEIK

SEQ ID NO: 156-164 - skipped (9 entries)

SEQ ID NO: 165 - protein, 111 aa, Artificial Sequence
  Description of Artificial Sequence: Synthetic polypeptide
QVQLKESGPGLVAPSQSLSITCTVSGFSLTSYGVSWVRQPPGKGLEWLGVIWGDGSTNYHSTLISRLSISKDNSKSQVFLKLNSLQTDDTATYYCARVAYWGQGTLVTVSA

SEQ ID NO: 166 - protein, 5 aa, Artificial Sequence
  Description of Artificial Sequence: Synthetic peptide
QYNST

SEQ ID NO: 167 - protein, 372 aa, Homo sapiens
MNYPLTLEMDLENLEDLFWELDRLDNYNDTSLVENHLCPATEGPLMASFKAVFVPVAYSLIFLLGVIGNVLVLVILERHRQTRSSTETFLFHLAVADLLLVFILPFAVAEGSVGWVLGTFLCKTVIALHKVNFYCSSLLLACIAVDRYLAIVHAVHAYRHRRLLSIHITCGTIWLVGFLLALPEILFAKVSQGHHNNSLPRCTFSQENQAETHAWFTSRFLYHVAGFLLPMLVMGWCYVGVVHRLRQAQRRPQRQKAVRVAILVTSIFFLCWSPYHIVIFLDTLARLKAVDNTCKLNGSLPVAITMCEFLGLAHCCLNPMLYTFAGVKFRSDLSRLLTKLGCTGPASLCQLFPSWRRSSLSESENATSLTTF

SEQ ID NO: 168 - protein, 368 aa, Homo sapiens
MVLEVSDHQVLNDAEVAALLENFSSSYDYGENESDSCCTSPPCPQDFSLNFDRAFLPALYSLLFLLGLLGNGAVAAVLLSRRTALSSTDTFLLHLAVADTLLVLTLPLWAVDAAVQWVFGSGLCKVAGALFNINFYAGALLLACISFDRYLNIVHATQLYRRGPPARVTLTCLAVWGLCLLFALPDFIFLSAHHDERLNATHCQYNFPQVGRTALRVLQLVAGFLLPLLVMAYCYAHILAVLLVSRGQRRLRAMRLVVVVVVAFALCWTPYHLVVLVDILMDLGALARNCGRESRVDVAKSVTSGLGYMHCCLNPLLYAFVGVKFRERMWMLLLRLGCPNQRGLQRQPSSSRRDSSWSETSEASYSGL

SEQ ID NO: 169 - protein, 116 aa, Mus sp.; identical to SEQ ID NO: 8

SEQ ID NO: 170 - protein, 117 aa, Mus sp.; identical to SEQ ID NO: 24

SEQ ID NO: 171 - protein, 112 aa, Mus sp.; identical to SEQ ID NO: 40

SEQ ID NO: 172 - protein, 111 aa, Mus sp.; identical to SEQ ID NO: 56

SEQ ID NO: 173 - protein, 111 aa, Mus sp.; identical to SEQ ID NO: 165

SEQ ID NO: 174 - protein, 117 aa, Mus sp.; identical to SEQ ID NO: 75

SEQ ID NO: 175 - protein, 112 aa, Mus sp.; identical to SEQ ID NO: 16

SEQ ID NO: 176 - protein, 112 aa, Mus sp.; identical to SEQ ID NO: 32

SEQ ID NO: 177 - protein, 111 aa, Mus sp.
DIVLTQSPASLTVSLGQRATISCRASESVDNYGISFMNWFQQKPGQPPKLLIYAASNQGSGVPARFSGSGSGTDFSLNIHPMEEDDTAMYFCQQSKEVPWTFGGGTKLEIK

SEQ ID NO: 178 - protein, 112 aa, Mus sp.; identical to SEQ ID NO: 63

SEQ ID NO: 179 - protein, 112 aa, Mus sp.; identical to SEQ ID NO: 68

SEQ ID NO: 180 - protein, 112 aa, Mus sp.; identical to SEQ ID NO: 83

SEQ ID NO: 181 - protein, 111 aa, Artificial Sequence; identical to SEQ ID NO: 88
  Description of Artificial Sequence: Synthetic polypeptide

SEQ ID NO: 182 - protein, 111 aa, Artificial Sequence; identical to SEQ ID NO: 92
  Description of Artificial Sequence: Synthetic polypeptide

SEQ ID NO: 183 - protein, 111 aa, Artificial Sequence; identical to SEQ ID NO: 96
  Description of Artificial Sequence: Synthetic polypeptide

SEQ ID NO: 184 - protein, 112 aa, Artificial Sequence; identical to SEQ ID NO: 101
  Description of Artificial Sequence: Synthetic polypeptide

SEQ ID NO: 185 - protein, 112 aa, Artificial Sequence; identical to SEQ ID NO: 137
  Description of Artificial Sequence: Synthetic polypeptide

SEQ ID NO: 186 - protein, 112 aa, Artificial Sequence; identical to SEQ ID NO: 106
  Description of Artificial Sequence: Synthetic polypeptide

SEQ ID NO: 187 - protein, 112 aa, Artificial Sequence; identical to SEQ ID NO: 109
  Description of Artificial Sequence: Synthetic polypeptide